(19) United States
(12) Patent Application Publication
(10) Pub. No.: US 2021/0308196 A1
(43) Pub. Date: Oct. 7, 2021

(54) COMPOSITIONS AND METHODS TO PROMOTE HOST DEFENSE AND STIMULATE, EXPAND, AND/OR RESET T CELL REPERTOIRES

(71) Applicant: EVOLVE BIOSYSTEMS, INC., Davis, CA (US)

(72) Inventors: Steven A. FRESE, Davis, CA (US); Bethany HENRICK, Davis, CA (US)

(21) Appl. No.: 15/733,936

(22) PCT Filed: Jun. 3, 2019
(86) PCT No.: PCT/US2019/035136

§ 371 Date: Dec. 1, 2020

Related U.S. Application Data
(60) Provisional application No. 62/679,739, filed on Jun. 1, 2018.
(60) Provisional application No. 62/730,517, filed on Sep. 12, 2018.

(51) Int. Cl. A61K 35/745; A61K 31/07; A61K 31/198; A61K 31/702; A61K 47/26; A61K 35/20; A61K 9/00; A61P 37/04; A61P 11/06; A61P 1/14; A61P 37/08; A61K 31/05; A23L 33/155; A23L 33/125; A23L 33/135; A23L 33/00; A23L 33/175; A23K 20/174; A23K 20/163; A23K 10/18; A23K 20/142

(52) U.S. Cl. CPC A61K 35/745; A23V 2002/00; A61K 31/198; A61K 31/702; A61K 47/26; A61K 35/20; A61K 9/0056; A61P 37/04; A61P 11/06; A61P 1/14; A61P 37/08; A61K 31/05; A23L 33/155; A23L 33/125; A23L 33/135; A23L 33/40; A23L 33/175; A23K 20/174; A23K 20/163; A23K 10/18; A23K 20/142; A23Y 2300/45; A61K 31/07

(57) ABSTRACT

Compositions comprising vitamin A, vitamin D, threonine, mammalian milk oligosaccharides, and Bifidobacterium, B. infantis cell wall components and their uses to treat or prevent diseases, support therapies including metabolic and autoimmune diseases.

### CROSS REFERENCE TO RELATED APPLICATIONS

This application is a National Stage Entry of PCT/US2019/035136, filed Jun. 3, 2019, which claims priority to U.S. Provisional Application No. 62/730,517 filed on Sep. 12, 2018 and U.S. Provisional Application 62/679,739 filed on Jun. 1, 2018, the disclosures of each of which are hereby incorporated by reference in their entireties.

### FIELD OF INVENTION

The inventions described herein relate generally to promoting host defense through compositions and methods that modulate the gut microbiome and/or biochemistry to improve barrier function and B or T immune cell pathways within the immune system. Compositions and methods that modulate immune cell function comprise various combinations that may include one or more of Vitamin A or its derivatives, Bifidobacterium species, B. infantis in different forms that may including activating B. infantis, or its cell wall components whether the cell is live or dead, Vitamin D, threonine and/or oligosaccharides that when administered to the intestine of animals, particularly humans in need of stimulating the naïve or mature immune system, improve a condition such as immune immaturity, immune dysfunction or direct immune function stimulation to improve specific immunotherapies.

### BACKGROUND

The gut microbiome and its function is increasingly being recognized as a critical part in health and disease, and critical to the proper function of the immune system. The gastrointestinal tract or gut is exposed to a large number of antigens, including bacteria and food, every day. There are multiple layers of host defense, including the physical barrier of the intestinal epithelium, the composition of the gut microbiome, and both the innate and acquired immune systems.

B cells are lymphocytes that are part of the antigen recognition pathway that lead to antibody production and are part of the acquired immunity, while T regulatory (Treg) cells are a specialized CD4+ T-cell lineage that play an important role in maintaining self-tolerance. The dysfunction of these cells can be implicated in the development of various autoimmune and allergic diseases.

Retinoic acid, a vitamin A metabolite, regulates a wide range of biological processes, including cell differentiation and proliferation. Recent studies demonstrate that retinoic acid also regulates the differentiation of T helper cells and Treg cells and has been shown to sustain Treg stability under inflammatory conditions. Lui et al (2015) Cellular & Molecular Immunology 12: 553-557.

An exhaustive list of more than 1,000 microbial species in the human microbiome was studied, and the study concluded that most bacteria do not have the ability to stimulate Tregs. It was discovered that the ability to stimulate Tregs was limited to 38 species in the Clostridia Class that have the ability to cause a robust induction of the Tregs in the colon, which in turn produces elevated levels of IL-10 in the colon [U.S. Patent Application Publication No. 2016/0193257]. In other experiments, Polysaccharide A (PSA) found on the external surface of bacteria (a molecule produced by the PSA locus of Bacteroides fragilis—a member of the Clostrida Class) or other synthetic zwitterionic polysaccharides have been used to stimulate Tregs to treat, prevent, or control inflammations and inflammatory conditions [U.S. Patent Publication No. 2016/0030464 and U.S. Patent Publication No. 2014/0072534].

In a mouse study, B. longum was found to reduce Peyer's patch gene expression of peptides associated with antigen presentation, TLR signaling, and cytokine production while increasing expression of genes associated with retinoic acid metabolism and induced T regulatory cells in adult murine allergy models. B. breve in infant mice had an effect on Foxp3+T Regulatory cells, but did not have a protective effect on respiratory or oral allergy [Lyons et al. (2010) Clinical and Experimental Allergy (40): 811-819].

### SUMMARY OF INVENTION

The present invention provides compositions for use in foods or therapeutic applications comprising components selected from Vitamin A or its derivatives, Bifidobacterium, B. infantis solute binding proteins, B. infantis exopolysaccharide components, oligosaccharides, bioavailable threonine and Vitamin D.

The invention provides for composition comprising Vitamin A, or a Vitamin A derivative or metabolite thereof, and an oligosaccharide (OS).

The vitamin A may be retinol, retinal, retinoic acid, a provitamin A carotenoid, or a combination thereof. The provitamin A carotenoid may be alpha-carotene, beta-carotene, gamma-carotene, xanthophyll beta-cryptoxanthin, or a combination thereof. The provitamin A carotenoid may be beta-carotene. The composition may comprise delivering 1-10,000 International Units vitamin A per day. The composition may comprise delivers 1-2,000 International Units vitamin A per day. The composition may comprise about 5, 10, 15, 20, 25, 30, 35, 40, 45, 50, 55, 60, 65, 70, 75, 80, 85, 90, 95, or 100 μmol/l vitamin A. The composition may comprise about 1-100, 5-50, 25-75, 10-100, 30-60, or 75-100 μmol/l vitamin A.

In any of the above embodiments, the oligosaccharide (OS) may comprise one or more oligosaccharides with 2 to 10 residues (DP2-10 oligosaccharides). The OS may be a mammalian milk oligosaccharide (MMO). The mammalian milk oligosaccharide (MMO) may comprise oligosaccharide molecules found in human milk oligosaccharides (HMO), bovine milk oligosaccharides (BMO), bovine colostrum oligosaccharides (BCO), goat milk oligosaccharides (GMO), or a combination thereof. The oligosaccharides can include the carbohydrate polymers found in mammalian milk, which are not metabolized by any combination of digestive enzymes expressed by mammalian genes. The oligosaccharides composition can include one or more of lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof. The oligosaccharides may include: (a) one or more Type II oligosaccharide core where representative species include LnNT; (b) one or more oligosaccharides containing the Type II core and GOS in 1:5 to 5:1 ratio; (c) one or more oligosaccharides containing the Type II core and 2FL in 1:5 to 5:1 ratio; (d) a combination of (a), (b), and/or (c); (e) one or more Type I oligosaccharide core where representative species include LNT (f) one or more Type I core and GOS in 1:5 to 5:1 ratio; (g) one or more Type I core and 2FL in 1:5 to 5:1 ratio; and/or (h) a combination of any of (a) to (g) that includes both a type I and type II core. Type I or type II may be isomers of each other. Other type II cores include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH).

In any of the foregoing embodiments, oligosaccharide may animal, fungal, crustacean, insect or plant. In some embodiments, the oligosaccharide may be a plant-derived oligosaccharide. The plant oligosaccharide may be from carrots, peas, broccoli, onions, tomatoes, peppers, rice, soy, wheat, oats, bran, oranges, cocoa, olives, apples, grapes, sugar beets, cabbage, corn, or a mixture thereof. The plant oligosaccharide may be pre-digested polysaccharides from orange peels, cocoa hulls, olive pomace, tomato skins, grape pomace, corn silage, or a mixture thereof. The plant-derived oligosaccharides may be between 2 and 10 sugar residues (DP2-DP10), between 3 and 10 sugar residues (DP3-DP10), between 5 and 10 sugar resides (DP5-DP10), or up to DP20. In some embodiments, The fungal, insect or crustacean polysaccharides may be predigested to produce oligosaccharides. In some embodiments, chitin or chitosan are treated to produce fragments of that may be N-acetylglucosamine or N-acetylgalactosamine (NAG) or the NAG polymers may be DP2 to DP20.

In any of the foregoing embodiments, the oligosaccharide may comprise galactooligosaccharide (GOS) or fructooligosaccharide (FOS) or xylooligosaccharide (XOS).

In any of the foregoing embodiments, oligosaccharide (OS) may comprise a human milk oligosaccharide (HMO) from any source.

In any of the foregoing embodiments, composition may provide a total dietary intake of oligosaccharide in an amount of 0.001-100 grams per day. The oligosaccharide may be in an amount of 1-20 grams, 3-20 grams, 5-10 grams, 10-40 grams per unit dose. The oligosaccharide may be in an amount of 10, 15, 20, 25, 30, 35, 40, 45, or 50 grams. The total grams per day may be delivered over multiple servings in a day or given as a bolus once a day in various embodiments.

In any of the foregoing embodiments, composition may further comprise a Bifidobacterium. The Bifidobacterium may be Bifidobacterium adolescentis, Bifidobacterium animalis, Bifidobacterium animalis subsp. animalis, Bifidobacterium animalis subsp. lactis, B. bifidum, Bifidobacterium breve, Bifidobacterium catenulatum, Bifidobacterium longum subsp. infantis, B. pseudocatanulatum, Bifidobacterium pseudolongum, or a combination thereof. The composition may comprise an activated Bifidobacterium. The B. longum may be B. longum subsp. infantis (B. infantis). In preferred embodiments, the B. infantis has a functional H5 cluster. The B. longum subsp. infantis may be activated B. longum subsp. infantis. The exopolysaccharide and solute binding proteins may be increased on the cell surface of the B. infantis. The Bifidobacterium may be B. breve. The B. breve may be activated B. breve.

In any of the foregoing embodiments, the composition may comprise Bifidobacterium in an amount of 0.1 million-500 billion Colony Forming Units (CFU) per gram of composition. The composition may comprise Bifidobacterium may be in an amount of 0.001-100 billion Colony Forming Units (CFU) 0.1 million to 100 million, 1 million to 5 billion, or 5-20 billion Colony Forming Units (CFU) per gram of composition. The Bifidobacterium may be in an amount of 0.001, 0.01, 0.1, 1, 5, 15, 20, 25, 30, 35, 40, 45, or 50 billion Colony Forming Units (CFU) per gram of composition. The Bifidobacterium may be in an amount of 5-20 billion Colony Forming Units (CFU) per gram of composition or 5-20 billion Colony Forming Units per gram of composition or 0.1 million to 100 million Colony Forming Units per gram of composition

Any embodiment of this invention may include but is not limited to increasing bioavailability of threonine, N-acetyl threonine or gamma-glutamyl threonine in the intestine.

In some embodiments, Vitamin D status is monitored. In some embodiments, vitamin D is added to an oil formulation. In some embodiments, Vitamin D and Bifidobacterium are in an MCT oil composition, optionally with Vitamin A. In preferred embodiments, the Bifidobacterium is B. infantis that is optionally activated. In some embodiments, the total dietary intake of Vitamin D is increased in a subject in need of treatment for any of the conditions described herein. In some embodiments, vitamin D are added to milk in the diet. Vitamin D may be in the form of drops, capsules, or powder.

The composition may further comprise isolated B. infantis activated cell membranes comprising exopolysaccharides and/or solute binding proteins. In some embodiments, the intact dead cell is delivered in the composition.

In any of the embodiments, the composition may be in the form of a dry powder or a dry powder suspending in an oil. The composition may be spray dried or freeze-dried. The composition may be freeze-dried in the presence of a cryoprotectant.

In any of the foregoing embodiments, the composition may further comprise a stabilizer. The stabilizer may be a flow agent. The stabilizer may be a cryoprotectant. The cryoprotectant may be glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, taurine, mammalian milk oligosaccharides, polysaccharides or a combination thereof.

Any of the foregoing embodiments, the composition can be administered in a food composition, such as mammalian milk, mammalian milk-derived product, mammalian donor milk, human milk product, infant formula, a milk replacer, an enteral nutrition product, and/or a meal replacer. The OS can be administered in a powder that may be in a sachet, stickpack, capsule, tablet, or it may be a liquid such as in a syrup form, or may be suspended in other liquids including non-aqueous solutions like oils or gels or pastes. Non-bacterial compositions may be in an aqueous solution. The aqueous solution may be sterile.

In any of the foregoing embodiments, the composition may be formulated as a unit dose medicament.

In any of the foregoing embodiments, the composition may be a pharmaceutical composition, dietary supplement, nutritional product, food product, probiotic, and/or prebiotic.

In any of the foregoing embodiments, the composition may be formulated as a capsule, packet, sachet, foodstuff, lozenge, tablet, optionally an effervescent tablet, enema, suppository, dry powder, dry powder suspended in an oil, chewable composition, syrup, or gel.

In any of the foregoing embodiments, the composition may further comprise an intact protein source or breakdown products rich in threonine, the free amino acid—threonine, N-acetylthreonine, gamma-glutamylthreonine, or a combination thereof.

The invention also provides for a nutritional product comprising the compositions described herein. The nutritional product may be a food product, dietary supplement, infant formula, or pharmaceutical product.

Information for practicing the methods described herein are further described in U.S. Provisional Patent Application No. 62/558,349. These methods can reduce dysbiois, the risk of a mammal developing autoimmune, inflammatory or metabolic disorders such as, but not limited to Juvenile Diabetes (Type I), obesity, Diabetes (Type II), asthma, atopy, inflammatory Bowel disease, Celiac Disease, food allergies, autism, as compared to a dysmetabolic mammal. It may be expected that the risk will be reduced by a statistically significant amount. For example, the risk may be reduced by 20%, 30%, 40%, 50%, 60%, 70%, 80%, or 90%.

Methods described herein can increase the function of the immune system in a mammal, such as improving vaccine response and/or mucosal innate or adaptive immunity, and/or improving the production and transfer of secretory IgA in the intestine of the mammal. It may be expected that the response will be improved by a statistically significant amount. For example, the response may be improved by 20%, 30%, 40%, 50%, 60%, 70%, 80%, or 90%.

Methods described herein can increase the function of the immune system in a mammal, such as improving effectiveness of immunotherapy, and/or improving the specificity and sensitivity of specific immunotherapeutics. It may be expected that the response will be improved by a statistically significant amount. For example, the response may be improved by 20%, 30%, 40%, 50%, 60%, 70%, 80%, or 90%.

The invention also provides for a method for preventing and/or treating an autoimmune disease comprising administering the compositions described herein.

In an embodiment, a method for elevating regulatory T-cell (Tregs) and/or B cells comprises administering retinoic acid, or a source thereof, an oligosaccharide (OS), and optionally Bifidobacterium, to a subject. In other embodiments, Vitamin A status is measured, and a vitamin A diet is recommended as a supplement to treatment with OS and optionally, Bifidobacterium adequately provided by the diet. In some embodiments, a treatment regime may involve a sequence involving different formulations that contain one or more of OS, Vitamin A and Bifidobacterium for an initiation phase and a maintenance phase.

In an embodiment, a method for preventing and/or treating an autoimmune disease comprising administering Vitamin A, or a Vitamin A derivative or metabolite thereof, or a source thereof, an oligosaccharide (OS), and optionally Bifidobacterium, to a subject. In some embodiments, vitamin A status is monitored systematically or in fecal samples to determine Vitamin A availability and treatment adjusted accordingly.

In an embodiment, a method for preventing and/or treating an allergy comprising administering Vitamin A, or a Vitamin A derivative or metabolite thereof, or a source thereof, an oligosaccharide (OS), and optionally Bifidobacterium, to a subject.

In an embodiment, a method for increasing the efficiency of antigen recognition in an animal comprising administering Vitamin A, or a Vitamin A derivative or metabolite thereof, or a source thereof, an oligosaccharide (OS), and optionally Bifidobacterium, to a subject. The efficiency of a gene therapy and/or a vaccine may be increased in a subject in need thereof.

In an embodiment, a method for maintaining the integrity of the alimentary canal mucosal membrane during chemotherapy comprising administering Vitamin A, or a Vitamin A derivative or metabolite thereof, or a source thereof, and an oligosaccharide (OS), optionally Bifidobacterium, to a subject.

In an embodiment, a method for preventing and/or treating an autoimmune disease comprising administering: (a) Vitamin A, or a Vitamin A derivative or metabolite thereof, or a source thereof; (b) oligosaccharide (OS); and (c) Bifidobacterium.

In an embodiment, a method for preventing and/or treating an allergy comprising administering: (a) Vitamin A, or a Vitamin A derivative or metabolite thereof, or a source thereof; (b) oligosaccharide (OS); and (c) Bifidobacterium.

In an embodiment, a method for protecting the intestinal barrier integrity during chemotherapy or radiation treatment comprising administering: (a) oligosaccharide (OS); (b) Bifidobacterium; and (c) protein enriched for threonine and/or threonine, N-acetyl threonine and/or gammaglutamylthreonine; and (d) optionally, Vitamin A or its derivative. In an embodiment, a method for maintaining the integrity of the alimentary canal mucosal membrane during chemotherapy comprising administering: (a) Vitamin A, or a Vitamin A derivative or metabolite thereof, or a source thereof; (b) oligosaccharide (OS); (c) Bifidobacterium; and (d) protein enriched for threonine and/or threonine, N-acetyl threonine and/or gammaglutamylthreonine.

In an embodiment, a method for stimulating T regulatory (Treg) cells comprising administering: (a) oligosaccharide (OS); (b) Bifidobacterium; and (c) optionally, Vitamin A or its derivative.

In an embodiment, a method for stimulating mucin production comprising administering: (a) oligosaccharide (OS); (b) Bifidobacterium; and (c) a protein enriched for threonine and/or threonine, N-acetyl threonine and/or gammaglutamylthreonine. In some embodiments, individuals known to have adequate fecal threonine fecal levels in their diet are provided a composition of (a) or (b) or (a) and (b). In some embodiments, individuals are monitored for fecal threonine

In any of the foregoing embodiments, the autoimmune disease may be inflammatory bowel disease or celiac disease. The inflammatory bowel disease (IBD) may be ulcerative colitis (UC) or Crohn's Disease. The subject may be suffering from a hyperinflammatory gut. The allergy may be a food allergy or atopy.

In any of the foregoing embodiments, the subject may be a mammal. The mammal may be a human, cow, pig, rabbit, goat, sheep, cat, dog, horse, llama, or camel. The mammal may be an infant. The mammal may be a nursing infant mammal. The subject may be a human.

In any of the foregoing embodiments, the vitamin A may be retinol, retinal, retinoic acid, a provitamin A carotenoid, or a combination thereof. The provitamin A carotenoid may be alpha-carotene, beta-carotene, gamma-carotene, xanthophyll beta-cryptoxanthin, or a combination thereof. The provitamin A carotenoid may be beta-carotene.

In any of the foregoing embodiments, oligosaccharide (OS) may comprise at least about 15%, at least 25%, at least 50%, at least 75% at least 95% of the subject's total dietary fiber.

In any of the foregoing embodiments, an elevation of the regulatory T-Cells (Tregs) results in the suppression of deleterious T-helper (Tₕ) cells. The elevation of the regulatory T-Cells (Tregs) results in a decrease in inflammatory markers. The inflammatory markers may be IL-8, IL-6, TNF-α, IL-10 INF gamma, INF alpha, or a combination thereof. The inflammatory markers may be decreased by at least 5%, 10%, 15%, 20%, 25%, 30%, 35%, 40%, 45%, 50%, 55%, 60%, 65%, 70%, 75%, 80%, 85% or 90%.

In any of the foregoing embodiments, subject may be already colonized by a Bifidobacterium species as measured by Bifidobacterium species CFU/gram of feces or CFU/μg DNA). The colonization by Bifidobacterium species in the subject may be increased by at least 1-10 CFU/gram of feces. The subject may be not colonized by a Bifidobacterium species as measured by Bifidobacterium species CFU/gram of feces.

In any of the foregoing embodiments, the dosage of retinoic acid, or a source thereof, oligosaccharide (OS), Bifidobacterium, or combinations thereof, may be in an amount effective to maintain the total Bifidobacterium level at least 10⁶, at least 10⁸, at least 10⁹ or at least 10¹⁰ CFU normalized per either gram of feces or μg DNA or more preferably greater than 10⁸. Alternatively, the relative abundance of Bifidobacteriaceae family in the microbiome makes up at least 10%, at least 20%, at least 30%, at least 50%, at least 60%, at least 70% at least 80% at least 90% of the total measurable microbiome. In some embodiments, the Bifidobacterium is B. infantis and an effective amount is maintained at greater than 10⁶, 10′^{,} 10⁸, 10⁹, or 10¹⁰ CFU normalized per either μg DNA or gram of feces, more preferably greater than 10⁸ CFU. In yet other alternatives, the metagenome is measured by shotgun sequencing and the abundance of genes including but not limited to Blon 2175, Blon 2176 and/or Blon 2177 are increased compared to individuals not receiving the compositions described herein.

In any of the foregoing embodiments, the Bifidobacterium may be administered to the subject on a daily basis comprising from 0.1 million to 500 billion CFU of bacteria/day. The Bifidobacterium may be administered on a daily basis can include from 1 billion to 100 billion CFU/day or from 5 billion to 20 billion CFU/day. The Bifidobacterium may be administered on a daily basis for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days out to 365 days. The Bifidobacterium may be administered on a daily basis for at least 1-5 days, 6-10 days, 11-15 days, 16-20 days, 21-25 days, 26-30 days, 100 days, or for at least 3 months, 3-6 months, greater than 6 months, and greater than 1 year.

In any of the foregoing embodiments, the oligosaccharide may be administered in a solid or liquid form. The oligosaccharide may be administered in an amount of from about 0.1-50 g/day. The oligosaccharide may be administered in an amount of from about 2-30 g/day or 3-10 g/day.

In any of the foregoing embodiments, a first composition comprising retinoic acid and an oligosaccharide may be administered to the subject. The first composition may be administered several times a day, optionally 1-6 times a day. The first composition may be administered for at least 1-365 days.

In any of the foregoing embodiments, a second composition comprising Bifidobacterium may be administered to the subject. The second composition may be administered daily. The second composition may be administered for at least 1-365 days.

In any of the foregoing embodiments, the first composition comprising retinoic acid, or a source thereof and an oligosaccharide may be administered to a subject followed by the second composition comprising Bifidobacterium.

In any of the foregoing embodiments, a third composition comprising retinoic acid, oligosaccharide, and Bifidobacterium may be administered to a subject.

In any of the foregoing embodiments, the Vitamin A, or a Vitamin A derivative or metabolite thereof, or a source thereof, may be administered several times a day for at least 1-30 days.

In any of the foregoing embodiments, the oligosaccharide may be administered several times a day for at least 1-30 days. In other embodiments, the oligosaccharide may be administered for at least 30 days, at least 60 days, at least 90 days, at least 180 days, at least 1 year, or as needed as part of a healthy diet.

In any of the foregoing embodiments, the Bifidobacterium may be administered on a daily basis for at least 1-30 days. The Vitamin A, or a Vitamin A derivative or metabolite thereof, or a source thereof, oligosaccharide, and Bifidobacterium are administered to the subject in a composition on a daily basis for at least 1-30 days. The Vitamin A, or a Vitamin A derivative or metabolite thereof, or a source thereof and oligosaccharide are administered to the subject several times a day for at least 1-30 days followed by Bifidobacterium on a daily basis for at least 1-30 days.

In any of the foregoing embodiments, the function of the immune system in the mammal may be enhanced subsequent to administration of said bacteria, said MMO, or both. The enhancement in the function of the immune system may be improving: the vaccine response, mucosal innate or adaptive immunity, and/or improving homeostasis of innate and adaptive immunity systemically. In some embodiments, fecal calprotectin is assessed and an increased level is a sign of dysbiosis.

In any of the foregoing embodiments, the function of the immune system may be demonstrated by altered B or T cell populations, more specifically increased T regulatory and B regulatory cell populations, enhanced antibody titers in response to a vaccine, improved mucus production or decreased mucin degradation, or increased secretory immunoglobulin A (sIgA) production in the gut leading to protection against pathogenic bacteria. The increase may be statistically significant. The increase may be about 5%, 10% 20%, 30%, 40, 50, 60, 70, 80, or 90% more preferably 5-20%, 20-40% over a baseline sample for said subject or compared to expected values for subjects not receiving the compositions described herein

In any of the foregoing embodiments, compositions may be used to develop and/or strengthen the intestinal barrier in which proinflammatory cytokines (i.e. TNFα, IL-1β, and IFNγ) are decreased, ZO-1 and occluding proteins are increased, or myeloperoxidase is decreased.

In an embodiment, a composition for elevating regulatory T-cells (Tregs) comprising the compositions described herein. In a further embodiment, a composition changes helper T cell populations including but not limited to Th17. In some embodiments, TReg cells are measured and increased. In yet other embodiments, changes in other T cells population including but not limited to Th1, Th2, Th17, Th9 or other T cell populations are measured. In some embodiments, a ratio of Treg/Th17 is increased. In yet other embodiments Th17 is decreased or TReg is increased.

In some embodiments, levels of fecal interleukin 17A (IL-17), IL-8, IL-22, IL-1β, IL-6, IL-22, TNFα, IL-1β, and IFNγ are decreased with any of the compositions of this inventions, or IL-17, IL-8, IL-22, IL-1β, IL-6, IL-22, TNFα, IL-1β, and IFNγ may be increased with dysbiosis. In some embodiments, a value of greater than 180 pg/mg, greater than 100 pg/mg IL-17 is indicative of dysbiosis. In some embodiments, IL-4 concentration greater than 15 pg/mg is indicative of dysbiosis. In some embodiments, IL-13 concentration greater than 400 pg/mg is indicative of dysbiosis.

In some embodiments, the adaptive immune system may be measured by evaluating B cells and B reg cells, sIgA production and/or vaccine response (including IgA mucosally and IgG1 and IgE systemically) through antibody titers.

In an embodiment, methods for preventing or treating symptoms of autoimmune diseases wherein the autoimmune disease may be selected from inflammatory bowel disease (IBD: includes crohn's disease, ulcerative colitis, Inflammatory Bowel syndrome), necrotizing enterocolitis (NEC), atopy, allergy, asthma, celiac disease, autism, type I diabetes comprising any of the compositions described herein.

In an embodiment, the subject is a pregnant women. In some embodiments, the pregnant woman is in her 3^{rd} or 4^{th} trimester.

In an embodiment for preventing or treating a metabolic disease wherein the disease or condition may be selected from obesity, type II diabetes or processes involved in cognitive development (learning, depression).

In one embodiment, a composition for supporting (adjuvant) a cancer treatment comprising the compositions described herein.

In one embodiment, a composition for maintaining the integrity of the alimentary canal mucosal membrane during chemotherapy or extreme chemical reduction of the microbiome in the case of recurrent Clostridium difficile (C. difficile) infections comprising the compositions described herein. In some embodiments, recurrent C. difficile or other refractive infections are treated with a composition described herein either pre or post fecal transplant.

In some embodiments, the target population is a human infant with a dysbiotic gut microbiome. In other embodiments, the composition improves vaccine responses and efficacy of immune system targeted therapies meant to improve health of an individual of any age.

All applications of this invention may be used for preventing and/or improving inappropriate responses to conditions resulting from pregnancy, birth, prematurity, colic, diaper rash, sleep, weaning onto complementary foods, weaning away from breast milk or formula onto solid foods, mucosal damage, atopic disease, food allergy, autoimmune diseases, metabolic conditions, cognitive development, obesity, pre or post fecal transplant therapy, gene therapy, immunotherapy or vaccine response.

### BRIEF DESCRIPTION OF THE DRAWINGS

FIG. 1. PCoA of the gut microbiome at the family level; control (CON) samples are shown as gray triangles, and EVC001-fed infant samples are shown as light gray circles. 87.5% of total variation was described in the first two principal components (PC1 and PC2). PERMANOVA comparisons identified a significant difference between the two treatment groups by composition (R=26.5, P=0.001).

FIG. 2. Comparison of fecal glycome and colonic mucin-derived O-glycans of control and EVC001-fed infant feces. (A) Total number of OS detected across treatment groups. (B) Number of colonic mucin-derived O-glycans across treatment groups. (C) Relative abundance of the total number of colonic mucin-derived O-glycans in the total OS pool across treatment groups. (D) Percent of the OS assigned to colonic mucin-derived O-glycans in the total OS abundance across treatment groups.

FIG. 3. PCoA of colonic mucin-derived O-glycan composition among samples using a Bray-Curtis dissimilarity index; control (Con) samples are shown as gray points, and EVC001-fed infant samples are shown as teal points. 63.3% of total variation was explained in the first two principal components (PC1 and PC2). PERMANOVA comparisons identified a significant difference between the two groups, with respect to colonic mucin-derived O-glycan composition (R=12.4; P=0.001).

FIG. 4. Relative abundance of specific gut taxa postnatally. Box plots represent top 10 most abundant gut taxa for Control and EVC001-fed infants at (a) Day 6, (b) Day 40, (c) Day 60. P-values were considered to be statistically significant if * P<0.05; ** P<0.01; *** P<0.001; **** P<0.0001.

FIG. 5A-B. Fecal calprotectin levels are dependent on the abundance of Bifidobacteriaceae. Forty fecal samples from Day 40 postnatal were evaluated for the concentration of fecal calprotectin and Bifidobacteriaceae abundance (A) and subdivided based on Bifidobacteriaceae abundance < or >25% (B). The data set is representative of at least three different experiments completed in duplicate and a non-parametric Wilcoxon rank sum test was used to determine significance with the corresponding P values adjusted and considered statistically significant if ****P<0.0001.

FIG. 6. Fecal cytokine signature of infants that received B. infantis EVC001. Radar representation of median cytokine concentrations [pg/mg] detected in fecal samples from the Controls (n=20) and infants fed Bifidobacterium infantis EVC001 (EVC001) on (a) Day 6 (Baseline), (b) Day 40 postnatal, and (c) Day 60 postnatal. Median values were adjusted to log scale, then normalized within each cytokine group from 0-1. Statistical analysis was completed using Wilcoxon rank sum test. P values were adjusted using Bonferonni-Holm method and considered statistically significant if * P<0.05; P<0.01.

FIG. 7 Fecal cytokine concentrations change postnatally. Box plots represent fecal proinflammatory cytokine concentrations [pg/mg] from the Controls (n=20) and EVC001-fed infants (n=20) at Day 6 (baseline), Day 40, and Day 60 for (a) IL-2, (b) IL-5, (c) IL-6, (d) IL-8, (e) IL-10, (f) IL-22, (g) TNFα, (h) IL1β, and (i) IFNγ. Cytokine concentrations were measured in duplicate using MesoScale Discoveries U-plex. Statistical analysis was completed using Wilcoxon rank sum test. P-values were adjusted using the Bonferroni-Holm method and considered to be statistically significant if * P<0.05; P<0.01; *** P<0.001; **** P<0.0001.

FIG. 8 Principal coordinates analysis (PCoA) of Global Cytokine Profiles according to Group Status. PCoA based on Bray-Curtis dissimilarity of global cytokine profiles between EVC001-fed infants and Controls at (A) Day 6 (baseline), (B) Day 40, (C) Day 60 postnatal.

FIG. 9 Correlations between specific gut taxa and intestinal inflammatory cytokine responses. Heatmap shows correlation between bacterial families and specific cytokines computed via Spearman correlation P-values are corrected using Benjamini-Hochberg procedure (FDR) to estimate significant correlations between microbial taxonomic composition and specific cytokine concentration detected in the feces of exclusively breastfed infants at three time points (Day 6 (baseline), Day 40, and Day 60 postnatal). Each cytokine was tested in duplicate at three different time points. P-values were adjusted and considered to be statistically significant if *P<0.05 (empty circle); **P<0.01 (semi-solid circle); ***P<0.001 (solid circle).

FIG. 10. The relationship between sIgA production and relative abundance of Bifidobacteriaceae

FIG. 11. Box plots represent fecal proinflammatory cytokine IL-17A, IL-13 and IL4 concentration [pg/mg} from the controls at Day 60 postnatal. Cytokine concentration were measured in duplicated using MesoScale Discoveries U-plex

FIG. 12A-D depicts the change in (A) spleen weights; (B) cecum weights; (C) lymphocyte counts in spleen; (D) lymphocyte counts in mesenteric lymph nodes (MLN) resulting from a treatment of humanized mice with LNnT alone or in combination with B. infantis.

FIG. 13A-F depicts the analysis of lymphocyte populations in untreated and treated mice (A) total lymphocytes; (B) CD4+; (C) CD4+/CD25+Helios− FoxP3+; (D) CD4+/CD25+/Helios− FoxP3−; (E) CD4+/FoxP3+CD25−; (F) CD4+/Helios+CD25−

FIG. 14 depicts the colony forming units of B. infantis and enterobacteria in mice after 21 days.

FIG. 15 depicts the treatment groups for the SAM trial in Example 12

FIG. 16 depicts the stratification of the subjects in the study described in Example 16

### DETAILED DESCRIPTION OF THE INVENTION

As described in International Application PCT/US2018/050973, creating a healthy intestinal environment is important for the overall health of the mammal. The inventors have discovered means of providing or removing key metabolites and/or their precursors in the intestine in amounts sufficient to change the overall intestinal metabolome. The abundance of key metabolites can act in nutritive, absorptive, metabolic and immunological functions to promote the overall health of the mammal. These metabolites can also be administered in a therapeutic capacity to restore homoeostasis in conditions of altered metabolic (i.e., obesity, Type 2 diabetes, necrotizing enterocolitis), cognitive function (i.e., cognitive development, learning, depression, autism), autoimmune (i.e., celiac disease, Type I diabetes, atopy, allergy) or inflammatory (Le, inflammatory bowel disease, irritable bowel syndrome).

These metabolites may be increased or decreased alone or in combination to modulate the physiology, immunology, and biochemistry of the infant gut, which is conceptually described in more detail in International Application PCT/US2018/050973. That application describes compositions, methods, and protocols to provide adequate levels of these compounds to restore and promote nutritional and metabolic health of the intestine, as well as the health of other key organs including the liver and central nervous system. Monitoring the status of the some or all of the metabolites may be used to identify persons at risk of developing diseases in the future.

Generally, the key components are delivered through administering a composition comprising retinoic acid or sources thereof and oligosaccharides (OS) that are mammalian milk oligosaccharides (MMO) or functional equivalents thereof to an animal and more specifically to a mammal and even more specifically to a human. These compositions may be administered in conjunction with a bacterial composition comprising bacteria expressing key exopolysaccharides on their cell surface and may be activated to utilize the OS in the composition.

### Formulations and Uses of Vitamin A or its Derivatives

In some embodiments, dietary Vitamin A is delivered as preformed Vitamin A or proVitamin A as part of the diet. In other embodiments, vitamin A is supplemented beyond their typical diet to increase vitamin A consumption. Preformed Vitamin A is described as being from meat, poultry, fish or dairy, while provitamin A is from plant sources. Vitamin A deficiency is rare in the United states; however can be a problem in premature infants and in lesser developed nations. In some embodiments of this invention the composition will contain about 2.3 μmol/l vitamin A. In other compositions will contain at least 1 μmol/l vitamin A and in other compositions vitamin A may range between 0.4-and 1.2 μmol/l). In yet other embodiments, the target vitamin A concentration for the subject is 6-10 μmol/L. Vitamin A (retinol) is ingested as either retinyl esters or carotenoids and metabolized to active compounds such as 11-cis-retinal, and all-trans-retinoic acid.

Retinoids are generally isolated from animal sources and carotenoids are isolated from plant sources. In some embodiments, the source of vitamin A is provided in the form of retinoic acid or other derivative and may be used to stimulate T regulatory cells (Tregs). In other embodiments, a precursor to retinoic acid from the retinoid family of compounds is provided. In other embodiments, the plant carotenoid is delivered with the bacterial composition and converted to retinoic acid or retinol by the intestinal microbiome. In yet other embodiments, alpha-carotene, beta-carotene, gamma-carotene, and beta-cryptoxanthin and astaxanthin are examples of plant carotenoids that are provided and may be converted to retinoic acid under certain conditions in the intestine whether it be from host genetic capacity or the microbiome. In some embodiments, sources of carotenoids are used. In other embodiments, sources of retinoids are used and in further embodiments, a combination of carotentoids and retinoids in 1:10 to 10:1 ratios to provide a means of controlling the availability of retinoic acid (e.g., time-released) to maintain a constant source of retinoic acid. In some embodiments, carotenoids are considered a slow release retinoic acid while retinol is considered a quick release or bioavailable source. In some embodiments, the composition is formulated to release retinoic acid in the small intestine and in other embodiments, the composition is formulated to release retinoic acid in the large intestine or colon. Vitamin A may be expressed in International Units. International units can be converted to mg vitamin A. Vitamin A or provitamin A may also be discussed in terms of retinol Activity equivalents (RAE). The present invention provides for intakes of vitamin A for people aged 14 years and older range between 700 and 900 micrograms (mcg) of retinol activity equivalents (RAE) per day, for women who are nursing range between 1,200 and 1,300 RAE per day, for infants and children under 3 from 1500-2500 IU, and for adults older than 19 from about 6,000-15,000 IU.

One aspect of this invention requires increased bioavailability of retinol and/or increased conversion to retinoic acid that may not be achieved with general recommended levels of Vitamin A for a particular age group or gender. The ability to stimulate, tolerize and/or expand the TReg population in a subject in need of such intervention may require a conditional increase of bioavailable Vitamin A sources, such as preformed Vitamin A and/or provitamin A to increase metabolic conversion to retinoic acid to meet the increased metabolic demand. In other embodiments, where individuals are known or expected to be Vitamin A deficient, preparation of the compositions will include calculating the requirements for individuals consuming a certain amount of preformed Vitamin A and/or provitamin A to meet or exceed a threshold in the diet of that individual. In other embodiments, the ratio of retinoids to carotenoids is determined, so as to provide a sustainable increase in retinoic acid in an individual. In yet other embodiments, serum levels of retinoic acid are monitored to achieve a constant state.

### Compositions and Formulations of Oligosaccharides

The OS composition (structures present) and their amount (grams) may support colonization and activation of B. infantis. The OS composition may maintain the activation of B. infantis.

The term “oligosaccharide” as used herein, refers broadly to any oligosaccharide having between 3 and 20 residues regardless of the source of the oligosaccharide.

A lacto-N-biose (LNB) is a moiety that is core to oligosaccharides or may be an entity itself. It may be in a type I or Type II core configuration meaning a beta 1-3 or beta 1-4 linkage, respectively. N-acetyl lactosamine is an example of a type II entity. LNnT is an example of a larger oligosaccharide structure that contains the Type II core. An example of a larger type I core is LNT.

The “source of the oligosaccharide,” as used herein refers broadly to oligosaccharides from animal, insect, crustacean, microbial, plant, fungi or algae or chemical synthesis that are free oligosaccharides, as well as those bound to animal or plant proteins or lipids (glycans), as well as those glycan structures after they are released from proteins or lipids or mixtures thereof.

The term “mammalian milk oligosaccharide” or MMO, as used herein, refers broadly to those indigestible glycans, sometimes referred to as “dietary fiber”, or the carbohydrate polymers that are not hydrolyzed by the endogenous mammalian enzymes in the digestive tract (e.g., the small intestine) of the mammal. Mammalian milks contain a significant quantity of MMO that are not usable directly as an energy source for the milk-fed mammal but may be usable by many of the microorganisms in the gut of that mammal. MMOs can be found as free oligosaccharides (3 sugar units or longer, e.g., 3-20 sugar residues) or they may be conjugated to proteins or lipids.

In some embodiments, the composition optionally comprises bacterial cell wall exopolysaccharides. In some embodiments, live cells are used to provide the exopolysacccharide. In other embodiments, dead cells are used to provide the exopolysaccharide. In further embodiments, a combination of live and dead cells is used.

The OS, including MMO and their functional equivalents such as, but not limited to, MMO separated from natural milks, synthetic nature-identical MMOs, modified plant or fungal polysaccharides, modified animal, insect or crustacean polysaccharides, or glycans released from animal or plant glycoproteins (i.e. milk, meat, egg, fish, soy, corn, peas) that support growth and metabolic activities of these bacteria and thus may be used in this invention.

Mammalian milk contains a significant quantity of mammalian milk oligosaccharides (MMO) as dietary fiber. For example, in human milk, the dietary fiber is about 15% of total dry mass, or approximately 15% of the total caloric content. These oligosaccharides comprise sugar residues in a form that is not usable directly as an energy source for the mammalian infant or adult or for most of the microorganisms in the gut of that mammal.

The MMO may be provided to the mammal in the form of a food composition. The food composition can include mammalian milk, mammalian milk derived product, mammalian donor milk, an infant formula, milk replacer, or enteral nutrition product, or meal replacer for a mammal including a human. In some embodiments, the addition of the bacterial composition and the food composition that includes MMO can occur contemporaneously, e.g., within less than 2 hours of each other.

The MMO used for this invention can include fucosyllactose (FL) or derivatives of FL including but not limited to, lacto-N-fucopentose (LNFP) and lactodifucotetrose (LDFT). They may be neutral such as but not limited to N-acetlylactosamine, Lacto-N-Biose (LNB), lacto-N-tetraose (LNT) and lacto-N-neotetraose (LNnT), which can be purified from mammalian milk such as, but not limited to, human milk, bovine milk, goat milk, or horse milk, sheep milk or camel milk, or produced directly by chemical synthesis. The composition can further comprise one or more bacterial strains with the ability to grow and divide using fucosyllactose or its derivatives thereof as the sole carbon source. Such bacterial strains may be naturally occurring or genetically modified and selected to grow on the fucosyllactose or its derivatives if they did not naturally grow on those oligosaccharides.

The MMO can also be sialyllactose (SL) or derivatives of SL such as, but not limited to, 3′ sialyllactose (3SL), 6′ sialyllactose (6SL), and disialyllacto-N-tetrose (DSLNT), which can be purified from mammalian milk such as, but not limited to, human milk, bovine milk, goat milk, or mare's milk, sheep milk or camel milk, or produced directly by chemical synthesis. The composition further comprises one or more bacterial strains with the ability to grow and divide using sialyllactose or derivatives thereof as the sole carbon source. Such bacterial strains may be naturally occurring or genetically modified and selected to grow on the sialyllactose or its derivatives, if they did not naturally grow on those oligosaccharides.

The MMO can be a mixture fucosyllactose (FL) or derivatives of FL and sialyllactose (SL) or derivatives of SL which are naturally found in mammalian milk such as, but not limited to, human milk, bovine milk, goat milk, and horse milk. The FL and SL or derivatives thereof may be found in a ratio from about 1:10 to 10:1.

Selective oligosaccharides (OS) as defined here are carbohydrates that are not digested by the mammal and favor the growth of particular bacteria over others. Selective oligosaccharides may be of mammalian milk, plant, algae, yeast origin provided they induce the desired metabolic profile. OS, as used herein, refers to those indigestible sugars of length DP2-DP20 from any source including plant, algae, yeast, or mammal. Oligosaccharides having the chemical structure of the indigestible oligosaccharides found in any mammalian milk are called OS herein, whether or not they are actually sourced from mammalian milk.

The OS can include one or more of lacto-N-biose, N-acetyllactosamine, lacto-N-triose, lacto-N-neotetrose, fucosyllactose, lacto-N-fucopentose, lactodifucotetrose, sialyllactose, disialyllactone-N-tetrose, 2′-fucosyllactose, 3′-sialyllactosamine, 3′-fucosyllactose, 3′-sialyl-3-fucosyllactose, 3′-sialyllactose, 6′-sialyllactosamine, 6′-sialyllactose, difucosyllactose, lacto-N-fucosylpentose I, lacto-N-fucosylpentose II, lacto-N-fucosylpentose III, lacto-N-fucosylpentose V, sialyllacto-N-tetraose, or derivatives thereof. In some embodiments, the OS contains a Type I core. In a preferred embodiment of the mixture, the OS contains a type II core. See, e.g., U.S. Pat. Nos. 8,197,872, 8,425,930, and 9,200,091, the disclosures of which are incorporated herein by reference in their entirety. Functional equivalents of MMO may include identical molecules produced using recombinant DNA technology described in, for example, Australia Patent Application Publication No. 2012/257395, Australia Patent Application Publication No. 2012/232727, and International Patent Publication No. WO 2017/046711.

In general, plant, and fungal fibers are large polysaccharide structures that can only be digested extracellularly by colonic bacteria that excrete certain hydrolases, followed by the ingestion of free sugar monomers or oligosaccharides produced by the extracellular hydrolysis. However, the enzymatic, chemical or biological treatment of plant and fungal fibers can reduce the size of the glycans to the size that could be utilized by certain bacterial that are capable of ingesting and deconstructing MMOs such as, but not limited to, B. longum and B. breve. In addition, this invention contemplates treatment by synthetically and/or recombinantly-produced hydrolases that mimic microbial carbohydrate hydrolases, such as GHS, GH13, GH92, GH29.

Chemical treatment of plant polysaccharides would include acid hydrolysis (sulfuric, hydrochloric, uric, triflouroacetic), or hydrolysis using acidic hydrophobic, non-aqueous, ionic fluids followed by separation of the oligosaccharides in a two-phase reaction with water. Kuroda et al. ACS Sustainable Chem. Eng. (2016) 4(6): 3352-3356. Polysaccharides or glycans attached to proteins or lipids can be released by enzymatic processes using N-linked and/or O-linked glycans.

In some embodiments, chitin or chitosan may be derived from crustacean or fungal sources (i.e. shrimp and shitake mushrooms) and may be processed to deliver structures of DP 2-20 for use in certain compositions.

The formulations may comprise at least 5%, 10%, 15%, 20%, 25%, 30%, 35%, 40%, 45%, 50%, 55%, 60%, 65%, 70%, 75%, 80%, 85%, 90%, or at least 95% of N-acetyl-D-lactosamine (dimer; Type II core typical in LNnT). For example, the formulation may comprise about 5%-95%, 10%-80%, 50%-75%, or 20%-60% of N-acetyl-D-lactosamine (dimer; Type II core typical in LNnT). Additionally, the formulations may comprise at least 5%, 10%, 15%, 20%, 25%, 30%, 35%, 40%, 45%, 50%, 55%, 60%, 65%, 70%, 75%, 80%, 85%, 90%, or at least 95% of Type I core HMO (Gal-(1,3)-beta-GlcNAc), synthesized by enzymes bearing homology to beta-3-galactosyltransferase 1 (B3GALT1) found in the human genome. For example, the formulation may comprise about 5%-95%, 10%-80%, 50%-75%, or 20%-60% of Type I core HMO (Gal-(1,3)-Beta-GlcNAc). An oligosaccharide not found in human milk, such as a dimer structure or other intermediate dimer, including biose—e.g., lacto-N-biose—found during the synthetic production of oligosaccharides, can be used. The formulations may comprise 5%, 10%, 15%, 20%, 25%, 30%, 35%, 40%, 45%, 50%, 55%, 60%, 65%, 70%, 75%, 80%, 85%, 90%, or 95% of lacto-N-triose I (Gal-(1,3)-beta-GlcNAc-(1,3)-Gal), or lacto-N-triose II (GlcNAc-(1,3)-Gal-(1,3)-beta-Glu) or lacto-N-neotetrose (Gal-(1,4)-beta-GlcNAc-(1,3)-Gal). For example, the formulation may comprise about 5%-95%, 10%-80%, 50%-75%, or 20%-60% of lacto-N-triose I (Gal-(1,3)-beta-GlcNAc-(1,3)-Gal), or lacto-N-triose II (GlcNAc-(1,3)-Gal-(1,3)-beta-Glu) or lacto-N-neotetrose (Gal-(1,4)-beta-GlcNAc-(1,3)-Gal). The MMO may provide 0.2 grams to 40 gram per day.

MMO or similar selective oligosaccharides used at percentages above 5%, 10%, 15%, 20%, 25%, 30%, 35%, 40%, 45%, 50%, 55%, 60%, 65%, 70%, 75%, 80%, 85%, 90%, 95% diluted in non-selective oligosaccharides such as, but not limited to galactooligosaccharides (GOS), fructoologosaccharides (FOS), Xylosoligosaccharides (XOS) or combinations thereof in percentages below 95%, 90%, 85%, 80%, 75%, 70%, 65%, 60%, 55%, 50%, 45%. 40%, 35%, 30%, 25%, 20%, 15%, 10%, 5%. These combinations provide degrees of increasing selectivity where the higher the proportion of MMO or sources of selective oligosaccharide structures, the greater the selectivity for certain bacteria such as, but not limited to B. longum subsp. infantis.

Modifying the oligosaccharide structure to increase sialylation (Sialyllactosamine) or fucosylation can further increase their selectivity. The formulation may comprise type II core dimers of lactosamine, and fucosylated and/or sialidated oligosaccharides as the selective oligosaccharide fraction, the remainder of which is made up with non-selective oligosaccharides or less selective oligosaccharides. The composition may be formulated to also include Vitamin A, Vitamin A derivative or metabolite in an amount adjusted relative to the OS content of the composition.

The term “synthetic” composition refers to a composition produced by a chemi-synthetic process and can be nature-identical. For example, the composition can include ingredients that are chemically synthesized and purified or isolated. This does not include compositions that are naturally synthesized.

Purification of the oligosaccharide can mean separating a component of milk from any other components or otherwise processing mammalian milk including expressing human milk to provide for example the foremilk which is partially skimmed, human donor milk, or other human milk products such as fortifiers.

The OS may be provided to the mammal directly or in the form of a food composition. The composition may further comprise a food, and the food can comprise partial or the complete nutritional requirements to support life of a healthy mammal, where that mammal may be, but is not limited to, an infant or adult. The food composition can include mammalian milk, mammalian milk derived product, mammalian donor milk, an infant formula, milk replacer, an enteral nutrition product, or meal replacer for a mammal including a human. The OS may be in the form of a powder or liquid (water-based or oil-based), gel or paste.

### Compositions and Formulations of Bifidobacterium

In any of the foregoing embodiments, composition may further comprise a Bifidobacterium. The Bifidobacterium may be Bifidobacterium adolescentis, Bifidobacterium animalis, Bifidobacterium animalis subsp. animalis, Bifidobacterium animalis subsp. lactis, B. bifidum, Bifidobacterium breve, Bifidobacterium catenulatum, Bifidobacterium longum subsp. infantis, B. pseudocatanulatum, Bifidobacterium pseudolongum, or a combination thereof. The composition may comprise an activated Bifidobacterium. The B. longum may be B. longum subsp. infantis (B. infantis). The B. longum subsp. infantis may be activated B. longum subsp. infantis. The exopolysaccharide and solute binding proteins may be increased on the cell surface of the B. infantis. The Bifidobacterium may be B. breve. The B. breve may be activated B. breve.

In any of the above embodiments, the bacteria can be Bifidobacterium longum subsp. infantis EVC001 as deposited under ATCC Accession No. PTA-125180; cells were deposited with the American Type Culture Collection at 10801 University Blvd, Manassas, Va. 20110 under the Budapest Treaty on the International Recognition of the Deposit of Microorganisms for the Purposes of Patent Procedure, the “Deposited Bacteria.”

Additionally, “Deposited Bacteria,” as used herein, refers to the isolated Bifidobacterium longum subsp. infantis EVC001, deposited with the ATCC and assigned Accession Number, and variants thereof, wherein said variants retain the phenotypic and genotypic characteristics of said bacteria and wherein said bacteria and variants thereof have LNT transport capability and comprise a functional H5 gene cluster comprising at least BLON2175, BLON2176, and BLON2177.

A “functional H5 cluster,” refers to a cluster of genes in Bifidobacterium responsible for the uptake and metabolism of human milk oligosaccharides. A functional H5 cluster comprises Blon 2175, Blon 2176, and Blon 2177. The H5 cluster comprises the following genes: Blon 2171, Blon 2173, Blon 2174, Blon 2175, Blon 2176, Blon 2177, and galT.

Activation is defined as a means of turning on a specific nutrient consumption phenotype (like the HMO phenotype in B. infantis) in bacteria during production of the bacteria, which are dried in that state, examples of which are included in International Patent Application Nos. PCT/US2015/057226, filed Oct. 23, 2015, and PCT/US2019/014097, filed Jan. 18, 2019.

The State of the External Surface of Activated B. infantis

The bacteria may be administered contemporaneously with the OS, or they may already be present in the mammalian gut. Unlike most gut flora, certain important Bifidobacterium such as, but not limited to, B. longum subsp. infantis and B. breve, can internalize oligosaccharides that may be up to 3-20 sugar moieties in length providing that those oligosaccharides have certain specific glycosidic linkages for which these Bifidobacterium have endogenous glycosyl hydrolases to deconstruct the oligosaccharides. The functional range may preferably be further limited to 2-10 sugar moieties. This characteristic makes these Bifidobacterium uniquely successful in colonizing the gut of the breast-fed infant, the oligosaccharides (denoted herein as MMOs) are the right size and right composition to be uniquely consumed by these bacteria alone. Such structures also found in the carbohydrate components of certain plant and animal glycoproteins. The inventors have also discovered that when these glycans are released from their respective glycoproteins, they too can be used as a mimic of MMOs. Such oligosaccharides are preferentially internalized and metabolized by such bacteria as a consequence of their unique genetic capacity to do so. The oligosaccharides may be found in mammalian milk but can also be synthetic or plant-derived as long as they have the ability to select for the specific organism that can provide nutritive components required for the growth and/or development of an infant mammal.

A specific Exopolysaccharide gene cluster was found in B. longum that produces a characteristic branched exopolysaccharide with an unusual moiety deoxy-L-talose that occurs in a dense coating completely covering this organism. Altmann et al. PLOS one (2016) 11(9): e0162983. This same gene cluster is not present in B. infantis six of the required genes to make this structure are absent in B. infantis ATCC 15697 (Table 1). Table 2, describes a BLAST comparison of selected proteins between the two bifidobacteria species as a strategy to identify cell surface components that are unique to B. infantis. In table 2: pident=percent identity amongst the two aminoacidic sequences as computed by BLAST, Length=Length of the alignment in aminoacid, Mismatch=Number of mismatches between the two sequences, Gap open=Number of gap opened by BLAST to obtain optimal alignment, qstart=Start of the alignment in the query sequence, Qend=End of the alignment in the query sequence, Sstart=Start of the alignment in the subject sequence. In some embodiments, a B. infantis specific exopolysaccharide is expressed. In some embodiments, the cell membrane of B. infantis are used in compositions of this invention.

TABLE 1
Deoxy-L-talose Gene Cluster

| Protein ID | Protein name | Gene name |
|---|---|---|
| A0A1D7MRV3_BIFLN | NAD dependent epimerase/dehydratase family | B624_0350 |
| A0A1D7MRK3_BIFLN | Serine acetyltransferase (EC 2.3.1.30) | B624_0351 |
| A0A1D7MRW2_BIFLN | Polymerase involved in polysaccharide synthesis | B624_0353 |
| A0A1D7MRV0_BIFLN | Rhamnosyl transferase | B624_0354 |
| A0A1D7MRW3_BIFLN | Flippase protein involved in polysaccharide biosynthesis | B624_0355 |
| | dTDP-4-dehydrorhamnose 3,5-epimerase/dTDP-4-dehydrorhamnose | rmlC rmlD |
| A0A1D7MRL5_BIFLN | reductase | B624_0361 |

TABLE 2
complete comparison of B. longum vs B. infantis

| | Protein name B. | B. Infants | Protein name B. infants | | | | | | | |
|---|---|---|---|---|---|---|---|---|---|---|
| B. longum 35624 | longum 35624 | ATCC15697 | ATCC15697 | pident (%) | Length | mismatch | gapopen | qstart | qend | sstart |
| tr\|A0A1D7MRW1\|A0A1D7MRW1_BIFLN | Uncharacterized | tr\|B7GPQ1\|B7GPQ1_B1 | Uncharacterized protein | 97.22 | 144 | 4 | 0 | 1 | 144 | 1 |
| | protein OS = Bifid | | OS = Bifidobacterimn longum s | | | | | | | |
| tr\|A0A1D7MRX2\|A0A1D7MRX2_BIFLN | Uncharacterized | tr\|B7GUN5\|B7GUN5_B | Uncharacterized protein | 93.62 | 47 | 3 | 0 | 1 | 47 | 1 |
| | protein OS = Bifid | | OS = Bifidobacterimn longum st | | | | | | | |
| tr\|A0A1D7MRW0\|A0A1D7MRW0_BIFLN | dTDP-glucose | tr\|B7GNT9\|B7GNT9_BI | dTDP-glucose 4.6-dehydratase | 75.6 | 336 | 82 | 0 | 5 | 340 | 6 |
| | 4,6-dehydratase OS | | OS = Bifidobacterlum long | | | | | | | |
| tr\|A0A1D7MRJ5\|A0A1D7MRJ5_BIFLN | UDP-glucose | tr\|B7GNT8\|B7GNT8_B | UDP-glucose/GDP-mannose | 62.21 | 426 | 133 | 5 | 7 | 416 | 1 |
| | 6-dehydrogenase OS | | dehydrogenase OS = bifidob | | | | | | | |
| tr\|A0A1D7MRU2\|A0A1D7MRU2_BIFLN | Chain length | tr\|B7GK0\|B7GUK0_B | Lipopolysaccharide biosynthesis | 50.31 | 477 | 229 | 4 | 13 | 487 | 1 |
| | determinant protein | | OS = Bifidobacterimn lo | | | | | | | |
| tr\|A0A1D7MRJ1\|A0A1D7MRJ1_BIFLN | Glycosyl transferase | tr\|B7GUN0\|B7GUN0_B | Undecaprenyl-phosphate | 43.06 | 490 | 270 | 7 | 45 | 550 | 4 |
| | CpsD OS = Bif | | galactose phosphotransferase | | | | | | | |
| tr\|A0A0A1GQG3\|A0A0A1GQG3_BIFLN | Phosphotyrosine | tr\|B7GUM8\|B7GUM8_{—} | Protein tyrosine phosphatase | 34.95 | 186 | 112 | 2 | 1 | 185 | 1 |
| | protein phospha | | OS-Bifidobacterium long | | | | | | | |
| tr\|A0A1D7MRV9\|A0A1D7MRV9_BIFLN | Acetyltransferase | sp\|B7GSX2\|GLMU_BIFI | Bifunctional protein GImU | 34.15 | 41 | 27 | 0 | 87 | 127 | 402 |
| | OS = Bifidobacte | | OS = Bifidobacterium longum | | | | | | | |
| tr\|A0A1D7MRK1\|A0A1D7MRK1_BIFLN | Glycosyl transferase | tr\|B7GUM1\|B7GUM1_{—} | Capsular polysaccharide synthesis | 33.89 | 239 | 149 | 5 | 100 | 332 | 77 |
| | OS = Bifidoba | | OS = Bifidobacterium | | | | | | | |
| tr\|A0A1D7MRV2\|A0A1D7MRV2_BIFLN | Uncharacterized | tr\|B7GTR6\|B7GTR6_Bi | Uncharacterized protein | 28.82 | 569 | 354 | 17 | 91 | 643 | 43 |
| | protein OS = Bifid | | OS = Bifidobacterium longum s | | | | | | | |
| tr\|A0A1D7MRV4\|A0A1D7MRV4_BIFLN | Glycosyl | tr\|B7GRX8\|B7GRX8_Bi | Glycogen synthase | 26.58 | 316 | 195 | 13 | 99 | 380 | 96 |
| | transferase, group | | OS = Bifidobacterium longum | | | | | | | |
| | 1fam | | subsp. | | | | | | | |
| tr\|A0A1D7MRU7\|A0A1D7MRU7_BIFLN | UDP-glucuranate | tr\|B7GN76\|B7GN76_1Bi | UDP-gIucose 4-epimerase | 24.65 | 361 | 207 | 11 | 12 | 343 | 4 |
| | 5′-epimerase 0 | | OS = Bifidobacterium longum | | | | | | | |
| tr\|A0A1D7MRU9\|A0A1D7MRU9_BIFLN | Glycosyltransferase | tr\|B7GRX8\|B7GRX8_Bi | Glycogen synthase | 24.19 | 215 | 133 | 5 | 186 | 386 | 152 |
| | protein OS-B | | OS = Bifidobacterium longum | | | | | | | |
| | | | subsp. | | | | | | | |
| tr\|A0A1D7MRL3\|A0A1D7MRL3_BIFLN | Glucose-1-phosphate | sp\|B7GS87\|GLGC_BIFL | Glucose-1-phosphate adenylyltransferase | 24.11 | 282 | 150 | 16 | 3 | 236 | 9 |
| | thymidylyiti | | OS = Bifidoba | | | | | | | |
| tr\|A0A1D7MRV5\|A0A1D7MRV5_BIFLN | NAD-dependent | tr\|B7GNT9\|B7GNT9_Bi | dTDP-gIucose 4,6-dehydratase | 23.26 | 374 | 229 | 11 | 5 | 368 | 12 |
| | epimerase/dehyr | | OS- Bifidobacterium long | | | | | | | |
| tr\|A0A1D7MRY6\|A0A1D7MRY6_BIFLN | Glycosyl | tr\|B7GRX8\|B7GRXS_Bi | Glycogen synthase | 22.66 | 384 | 232 | 18 | 17 | 346 | 30 |
| | transferase, group | | OS- Bifidobacterium longum | | | | | | | |
| | 1 fam | | subsp. | | | | | | | |

In some embodiments, the composition comprises B. infantis with an overabundance of the Family 1 of solute binding proteins (F1SBPs). The inventors have discovered that when B. infantis is present in a form that expresses certain unique exopolysaccharides or Solute Binding Proteins, or a composition comprising key bacterial membrane components, a composition comprising oligosaccharides (OS) and retinoic acid fed to an individual will work synergistically to develop the immune system or reset an aberrant immune response and in particular promote expansion of and/or tolerance via T regulatory (TReg) cells.

In some embodiments, the exopolysaccharide layer specific to B. infantis and membrane components from a dead intact cell or a lysed cell membrane maybe included as part of the composition to increase the immune stimulation.

### Compositions Including Threonine

In some embodiments, higher levels of the amino acid threonine or N-acetylthreonine and/or the peptide gammaglutamylthreonine are included in the composition and measured in the feces of an individual consuming the composition and the level of mucin production may be greater than the level of mucin degradation. In other embodiments, the administration of the composition results in a thicker mucus layer on the cell surface. In some embodiments, the state of mucin degradation is monitored in the feces by looking for the amounts of certain mucin structures in the feces or it is monitored by the presence or absence of certain mucin degrading bacteria.

### Methods Used to Change the Immune System

Within the T cell population, changing the population of T regulatory (TReg) cells is an important component of the invention. A stronger intestinal barrier and/or appropriate B and T cell populations may increase efficacy of a given treatment for an infection or disease and/or may improve colonization of the gut microbiome and/or may reset and/or improve tolerization to food antigens. Appropriate T cell populations may include changes to Th1, Th2, Th17, Th9 or other T cell populations in addition to changes in TReg cells. TRegs also have the ability to suppress B cell and plasma cell responses leading to the suppression of B cell-mediated disease development, most notably autoimmunity. TRegs play an important role in controlling immune responses of B and T cells that are specific to self-antigens leading to autoimmunity. Furthermore, B regulatory cells (BRegs) have the ability to suppress CD4+ T cells.

IL-17A is one of six different cytokines including in the cytokine family IL-17 and is often referred to as just IL-17. It is predominantly expressed by a distinct type of T cells, T helper 17 (Th17) cells and can be expressed lesser by other specific lymphocytes, including Th17, NK T cells, macrophages, and Paneth cells to mediate pro-inflammatory responses and provide protective roles in host defense at epithelial and mucosal sites. Although IL-17 production is crucial for acute inflammation and protecting the host from pathogen invasion, chronic production of IL-17 can results in excessive pro-inflammatory cytokine expression and chronic inflammation, which lead to tissue damage and autoimmunity. IL-17 cytokines have been linked to many autoimmune diseases, including Multiple Sclerosis, Rheumatoid Arthritis, inflammatory bowel disease and psoriasis. In some cases, IL-4 or IL-13, IL-8, IL-22, IL-1β, IL-6, IL-22, TNFα, IL-1β, and IFNγ can be measured.

IL-17A can instigate and/or exacerbate fetal inflammatory responses that increase neonatal morbidities and mortalities of common neonatal conditions including sepsis, bronchopulmonary dysplasia, patent ductus arteriosus, and necrotizing enterocolitis. In some embodiments decreasing IL-17A production may decrease neonatal morbidity and mortality.

This invention includes but is not limited to increasing bioavailability of threonine, N-acetyl threonine or gamma-glutamyl threonine as further described in U.S. Provisional Patent Application No. 62/558,349 to facilitate mucus production, reducing mucin-degrading microbiome species. In some embodiments, one or more components are used as part of treatment regime that may vary in composition over time.

Methods described herein can increase the function of the immune system in a mammal such as improving vaccine response, tolerance to microbial and food antigens, and/or mucosal innate or adaptive immunity, and/or improving the production and transfer of secretory IgA in the intestine of the mammal. Increased function of the immune system is demonstrated for example by enhanced antibody titers in response to a vaccine, improved mucus production, increased T regulatory and B regulatory cell populations or increased sIgA production in the gut leading to protection against pathogenic bacteria. The increase in immune system response may expected to be statistically significant. For example, the response can be improved by 20%, 30%, 40%, 50%, 60%, 70%, 80%, or 90%.

The immune function may be selected for their ability to alter receptors like pattern recognition receptors (i.e., Toll like receptor 2 (TLR2), Toll like receptor 4 (TLR4), NOD-like receptorsfarnesoid X receptor (FXR), TGR5 or arylhydrocarbon receptor (AhR).

Immune modifications may include a decrease in COX-2.

### Age-Related States

Enhancement of the immune system through B cell development including but not limited to B cell maturation and plasma cell development is important during pregnancy, prematurity, infancy, colic, diaper rash, weaning, immunotherapy treatment and vaccine response in infants and older adults (55+).

### Disease States

A number of different autoimmune conditions, inflammatory conditions, and therapies requiring a functioning immune system may be improved by use of the compositions described herein including, but not limited to, inflammatory bowel disease (IBD) including Crohn's disease and ulcerative colitis and inflammatory bowel syndrome (IBS), necrotizing enterocolitis colitis (NEC), allergy, atopy, obesity, Type 1 Diabetes, Type II diabetes, vaccine responsiveness, autism, organ transplant, immunotherapy, and gene therapy.

IBD is an umbrella term to describe disorders that involve chronic inflammation of the gut. Treatment for IBD often requires anti-inflammatory or immune-suppressing drugs (with significant side effects) and/or surgery (with lifelong morbidity). Ulcerative colitis (UC), for example, is a condition that causes long-lasting inflammation and sores (ulcers) in the innermost lining of the large intestine (colon) and rectum, and over 50% of patients require surgery to remove the entire colon and rectum. There is a need for alternative, safer interventions to suppress the hyper-inflamed gut mucosa. Atopy and allergy are terms that encompass a number of conditions caused by hypersensitivity of the immune system to environmental allergens. These allergens are typically proteins that cause little to no problem in most people. The treatment for allergies includes the use of anti-histamines for mild allergies and intramuscular injections of epinephrine for those that suffer from more serious complications. Anaphylaxis, for example, is a serious allergic reaction that is rapid in its onset and can cause death. New treatments such as oral immunotherapy that introduces low levels of allergen in children known or expected to have a peanut allergy has shown promise to allow dietary reintroduction of peanuts. The present invention provides compositions that can be used for preventative or curative treatments of allergies that prevent or ameliorate the hypersensitive immune system and induce tolerance.

The Atopic March refers to the typical development and progression of allergic diseases early in life. These include atopic dermatitis (eczema), food allergy, atopic wheeze, asthma, and allergic rhinitis. It is also commonly referred to as the Allergic March.

Type I Diabetes, as known as Diabetes mellitus type 1, is a chronic metabolic disorder in which high levels of glucose are found in the blood leading to poor health outcomes. The present invention provides compositions that can be used for preventative measures to induce tolerance to avert autoimmune responses leading to destruction of insulin-producing cells in the pancreas.

Gene therapy and vaccine responses are often not tested given the strong efficacy in the past; however, it is now clear that gut microbiome composition has an impact on vaccine responses in infants and young children, which can make them more susceptible to morbidity and mortality. The present invention provides compositions that can be used for alternative, safer interventions to improve vaccine efficacy.

### Supplementation and Treatment Regimes

The compositions of this invention may be administered for at least 24 hours, at least 72 hours, at least 21 days, at least 28 days, at least 12 weeks, 16 weeks, 6 months, or at least 1 year to develop a robust and appropriate immune modification. The treatment is designed to stimulate the immune system for the purpose of improving host defense, including but not limited to improving mucus production and/or reducing mucus degradation, B cell responsiveness and/or expanding or altering the T Regulatory and Helper T cell profile. The composition may result in induction of oral tolerance and improved vaccine efficacy.

The compositions may be a food composition sufficient to provide partial or total source of nutrition for the mammal and may include a protein source rich in Threonine. The bacteria and the oligosaccharide, separately or in a food composition, are administered in amounts sufficient to maintain a desired level and composition of at least one metabolite in the mammal, e.g., increased metabolites such as threonine, N-acetyl threonine, or gamma glutamyl threonine and decreased metabolites such as retinol (Vitamin A). A complete list of metabolites can be found in U.S. Provisional Patent Application No. Ser. No. 62/558,349. Other examples of metabolites that change are found in International Patent Application No. PCT/US2017/040530, filed Jun. 30, 2017 and U.S. Provisional Patent Application No. 62/613,405, filed Jan. 3, 2017.

The following examples are provided to exemplify various modes of the invention disclosed herein, but they are not intended to limit the invention in any way.

### EXAMPLES

### Example 1

### Feeding B. infantis EVC001 to Infants Consuming HMO Rich Diet

This trial was designed to show the effect of probiotic supplementation with Bifidobacterium longum subsp. Infantis (B. infantis EVC001) in healthy, term, nursing infants compared to an unsupplemented group. A dry composition of lactose and activated Bifidobacterium longum subsp. infantis was prepared starting with the cultivation of a purified isolate (Strain EVC001 ATCC Accession No. PTA-125180, Evolve Biosystems Inc., Davis, Calif., isolated from a human infant fecal sample) in the presence of BMO according to International Patent Application No. PCT/US2015/057226. The culture was harvested by centrifugation, freeze dried, and the concentrated powder preparation had an activity of about 300 Billion CFU/g. This concentrated powder was then diluted by blending with infant formula grade lactose to an activity level of about 30 Billion CFU/g. This composition then was loaded into individual sachets at about 0.625 g/sachet and provided to breast-fed infants starting on or about day 7 of life and then provided on a daily basis for the subsequent 21 days.

This was a 60-day study starting with infants' date of birth as Day 1. Before postnatal day 6, women and their infants (delivered either vaginally or by cesarean-section), were randomized into an unsupplemented lactation support group or a B. infantis supplementation plus lactation support group. Infant birthweight, birth length, gestational age at birth, and gender were not different between the supplemented and unsupplemented groups. Starting with Day 7 postnatal, and for 21 consecutive days thereafter, infants in the supplemented group were given a dose of at least 1.8×10¹⁰ cfu of B. infantis suspended in 5 mL of their mother's breastmilk, once daily. Because the provision of HMO via breastmilk was critical for supporting the colonization of B. infantis, all participants received breast feeding support at the hospital and at home and maintained exclusive breast feeding through the first 60 days of life. Full study design is described in [Smilowitz et al. BMC Pediatrics (2017) 17:133 DOI 10.1186/s12887-017-0886-9].

Infant fecal samples were collected throughout the 60-day trial. Mothers collected their own fecal and breastmilk samples as well as fecal samples from their infants. They filled out weekly, biweekly and monthly health and diet questionnaires, as well as daily logs about their infant feeding and gastrointestinal tolerability (GI). Safety and tolerability were determined from maternal reports of infants' feeding, stooling frequency, and consistency (using a modified Amsterdam infant stool scale—watery, soft, formed, hard; Bekkali et al. 2009), as well as GI symptoms and health outcomes. Individual fecal samples were subjected to full microbiome analysis using Illumina sequencing based on 16S rDNA and qPCR with primers designed specifically for B. longum subsp. infantis strain.

B. infantis was determined to be well-tolerated. Adverse events reported were events that would be expected in normal healthy term infants and were not different between groups. Reports specifically monitored blood in infant stool, infant body temperature and parental ratings of GI-related infant outcomes such as general irritability, upset feelings in response to spit-ups and discomfort in passing stool or gas, and flatulence. Furthermore, there were no differences reported in the use of antibiotics, gas-relieving medications, or parental report of infant colic, jaundice, number of illnesses, sick doctor visits and medical diagnoses of eczema.

The B. infantis supplemented infants had a gut microbiome fully dominated (on average, greater than 70%) with B. longum subsp. infantis regardless of the birthing mode (vaginal or C-section). This dominance continued even after supplementation ended (Day 28) as long as the infant continued to consume breast milk indicating that B. infantis was colonizing the infant gut to levels higher than 10¹⁰ cfu/g feces. Furthermore, those infants that were colonized by the B. longum subsp. infantis also had much lower levels of proteobacteria and enterococci (including Clostridium and Escherichia species).

Unsupplemented infants (i.e., infants receiving the standard of care—lactation support but no supplementation of B. infantis) did not show B. infantis levels above 10⁶ cfu/g (i.e., the limit of detection) in their microbiome, and there were significant differences in the microbiomes between C-section and vaginally delivered infants. Eighty percent (8 of 10) unsupplemented infants delivered by C-section had no detectable Bifidobacterium species, and fifty-four percent (13 of 24) of the vaginally delivered infants had no detectable Bifidobacterium species by day 60. Further analysis of the thirteen unsupplemented infants that had some detectable Bifidobacterium, found that the species were primarily B. longum subsp. longum, B. breve and B. pseudocatenulatum. No detectable B. longum subsp. infantis was found in any of the unsupplemented infants in the study. Further analysis of the stool and other characteristics of differences between supplemented and unsupplemented infants is provided in Examples 1A-1F below and in International Application No. PCT/US2017/040530, incorporated herein by reference.

### Example 1A

### Metabolomic Analysis of Infant Feces

Fecal samples from infants of Example 1 were evaluated as described below to characterize the fecal metabolome and what effects colonization by this organism may have on the infant's metabolism as a whole.

Sample Preparation: Fecal samples were maintained at −80° C. until processed. Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. The resulting extract was divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent. The sample extracts were stored overnight under nitrogen before preparation for analysis.

Preparation of study-tracking replicates. A small aliquot of each sample was pooled to create a study tracking sample, which was then injected periodically throughout the platform run. Variability detected in the study tracking sample among consistently detected biochemicals can be used to calculate an estimate of overall process and platform variability.

Ultrahigh Performance Liquid Chromatography-Tandem Mass Spectroscopy (UPLC-MS/MS): All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions, however it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. Another aliquot was analyzed using basic negative ion optimized conditions using a separate dedicated C18 column. The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternated between MS and data-dependent MS' scans using dynamic exclusion. The scan range varied slighted between methods but covered 70-1000 m/z.

Data Extraction and Compound Identification: Raw data was extracted, peak-identified and QC processed using Proprietary hardware and software. Compounds were identified by comparison to library entries of purified standards or recurrent unknown entities in a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library +/−10 ppm, and the MS/MS forward and reverse scores between the experimental data and authentic standards. The MS/MS scores are based on a comparison of the ions present in the experimental spectrum to the ions present in the library spectrum.

Metabolite Quantification and Data Normalization: Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”). For studies that did not require more than one day of analysis, no normalization is necessary, other than for purposes of data visualization.

Determining the absolute concentration of metabolites in a fecal sample: Once the fecal samples was analyzed for the breadth of metabolites that changed in dysmetabolic infant fecal samples compared to the fecal samples taken from an infant treated with a composition from this invention, a series of known standards were assembled to help determine the absolute concentrations of certain metabolites using liquid chromatography-QTRAP or gas chromatography-quadrupole mass spectrometry. A standard curve is generated for known concentrations of a metabolite using the identified standards and the standard curve is used to determine the concentration of the metabolite in the fecal samples.

### Results

Fecal Samples from 20 infants supplemented with B. longum subsp. infantis (intervention) and 20 infants that were not supplemented (control) were analyzed for the levels of 983 metabolites. Analysis of 983 detected metabolites generated the major findings described in detail in International Patent Application No. PCT/US2018/050973 filed on Sep. 13, 2018.

### Reduced Levels of Retinol in the Gut of Human Infant

An example of a metabolite that is significantly reduced following administration of human milk and B. infantis compared to control is vitamin A (retinol) utilization. The vitamin A intake from breast milk was approximately the same between control and B. infantis EVC001 supplemented infants. However, in the feces Retinol was significantly reduced in B. infantis EVC001-treated infants from Example 1.

### Elevated Levels of Gamma-Glutamyl Cysteine and Creatinine in the Gut of a Human Infant

Creatinine and gamma-glutamyl cysteine and other gamma-glutamyl amino acids are important for preventing and/or recovering from oxidative stress. Gamma-glutamyl cysteine is an important precursor for glutathione (GSH). It is an integral part of preventing oxidative stress in a mammal. Creatinine is an important metabolite to reduce the effects of oxidative stress and can be instrumental in preventing oxidation mediated mitochondrial damage in premature and high risk deliveries. Oxidative stress is a condition that occurs during the birthing process. In term infants, GSH is generally sufficient, but it may not be in preterm infants, and it may also be low in people with autism.

Autism is a spectrum of disorders and is best treated early in life to minimize the severity. Diagnosis generally occurs after some critical windows have closed. Monitoring levels and recovery from oxidative stress during pregnancy and at birth may be an overall indicator of health and can be a tool to minimize long-term sub-clinical effects of early oxidative stress by administering the compositions in this invention.

An untargeted metabolomics analysis was completed on fecal samples collected in Example 1 from 20 infants at day 28 who were receiving the standard of care. The same analysis was completed on samples collected in Example 1 from 20 newborn infants receiving a composition of B. infantis and human milk oligosaccharides. The relative abundance of glutamyl-dipeptide metabolites were analyzed, and the results are reported in Table 5 below.

TABLE 3
significant changes in the gamma-glutamyl amino acids.

| Metabolites | Active/Control | P-value |
|---|---|---|
| gamma-glutamylalanine | 5.9 | 7.532E−08 |
| gamma-glutamylcysteine | 44.3 | 1.17E−12 |
| gamma-glutamylglutamate | 3.1 | 0.004279002 |
| gamma-glutamylglutamine | 1.6 | 0.001325298 |
| gamma-glutamylhistidine | 10.5 | 6.88631E−05 |
| gamma-glutamylisoleucine* | 8.1 | 8.144E−07 |
| gamma-glutamylleucine | 1.6 | 0.025584145 |
| gamma-glutamyl-alpha-lysine | 3.3 | 0.052765195 |
| gamma-glutamyl-epsilon-lysine | 2.4 | 1.66338E−05 |
| gamma-glutamylmethionine | 19.3 | 8.75E−09 |
| gamma-glutamylphenylalanine | 4.2 | 5.67663E−05 |
| gamma-glutamylthreonine | 7.15 | 1.4143E−07 |
| gamma-glutamyltyrosine | 2.8 | 0.000714584 |
| gamma-glutamylvaline | 3.3 | 1.11065E−05 |
| creatinine | 1.4 | 0.021567567 |

Table 3 shows significant changes in the gamma-glutamyl amino acids. The bold values are significant. The p-value is noted in column 3. A value above 1 means it is increased in Intervention compared to control while a number below 1 means it is decreased in intervention compared to control. The numerical value is ratio of active:control or the fold-change in the metabolite resulting from the treatment.

Creatinine and/or gamma-glutamyl cysteine can be used as metabolic indicators for monitoring levels pre and post-intervention and/or determining the need for an intervention to improve the health of said infant.

### Increased Levels of Threonine in the Gut of a Human Infant

Among those metabolites that significantly change between supplementation/intervention (Int) and control (con) group are threonine and its metabolites. There is more bioavailable threonine in infants fed human milk oligosaccharides and B. infantis EVC001. See International Patent Application No. PCT/US2018/050973 filed on Sep. 13, 2018.

TABLE 4
Changes in Threonine Levels

| | | Int/Con with |
|---|---|---|
| | Int/con | outlier removed |
| Threonine | 3.21 | 4.35 |
| N-acetyl Threonine | 3.07 | 3.67 |
| Gamma-glutamyl Threonine | 5.35 | 7.15 |

### Example 1B

### Increased Mucin Production

In this example, the mucin degradation was significantly less in the EVC001 supplemented infants compared to the control group. The following mucin structures were monitored as part of the metabolome in the stool of infants.

TABLE 5
Changes in Mucin Structures

| | | | Log₁₀ Volume | | Log₁₀ Volume | | |
|---|---|---|---|---|---|---|---|
| | | | Control | | EVC001-fed | | |
| Glycan | | Neutral | [Mean (+/− | | [Mean (+/− | | Holm- |
| Code | Composition | Mass | SD)] | | SD)] | | Sidak adjusted P value |
| 1_0_0_1 | 1HexNAc- | 512 | 7.19 | (+/−7.08) | 5.39 | (+/−5.54) | 0.0059 |
| | 1NeuAc | | | | | | |
| 1_1_0_1 | 1HexNAc- | 675 | 5.96 | (+/−6.01) | 4.01 | (+/−4.51) | 0.0704 |
| | 1Hex- | | | | | | |
| | 1NeuAc | | | | | | |
| 2_0_0_1 | 2HexNAc- | 716 | 5.93 | (+/−6.16) | 0 | (+/−0) | 0.2161 |
| | 1NeuAc | | | | | | |
| 2_1_1_0 | 2HexNAc- | 735 | 6.17 | (+/−6.23) | 0 | (+/−0) | 0.0704 |
| | 1Hex-1Fuc | | | | | | |
| 2_1_0_1 | 2HexNAc- | 878 | 5.9 | (+/−6.11) | 5.56 | (+/−5.58) | 0.4875 |
| | 1Hex- | | | | | | |
| | 1NeuAc | | | | | | |
| 2_1_2_0 | 2HexNAc- | 879 | 6.45 | (+/−5.95) | 5.48 | (+/−5.48) | <0.0001 |
| | 1Hex-2Fuc | | | | | | |
| 3_1_1_0 | 3HexNAc- | 936 | 6.43 | (+/−6.31) | 5.43 | (+/−5.49) | 0.0115 |
| | 1Hex-1Fuc | | | | | | |
| 2_1_1_1 | 2HexNAc- | 1024 | 7.31 | (+/−7.01) | 5.38 | (+/−5.53) | 0.0001 |
| | 1Hex-1Fuc- | | | | | | |
| | 1NeuAc | | | | | | |
| 2_1_1_2 | 2HexNAc- | 1315 | 7.08 | (+/−7.26) | 6.68 | (+/−7) | 0.4875 |
| | 1Hex-1Fuc- | | | | | | |
| | 2NeuAc | | | | | | |
| 3_1_0_2 | 3HexNAc- | 1372 | 6.43 | (+/−6.52) | 5.47 | (+/−5.65) | 0.1384 |
| | 1Hex- | | | | | | |
| | 2NeuAc | | | | | | |
| 3_1_2_1 | 3HexNAc- | 1373 | 7.52 | (+/−7.28) | 6.12 | (+/−6.38) | 0.0006 |
| | 1Hex-2Fuc- | | | | | | |
| | 1NeuAc | | | | | | |

A library of known mucin-specific O-glycans was compiled and used to query untargeted mass spectra of fecal samples. It was hypothesized that the modification of the gut microbiome resulted in modulation of mucin degradation by gut microbes. A second part of the hypothesis was that colonization with B. infantis, which does not degrade mucin, and the subsequent reduction of mucolytic taxa would diminish mucin degradation, as measured by the abundance of mucin-specific O-glycans in the infant stool.

Analysis of spectra obtained with nano-high performance liquid chromatography-chip/time-of flight mass spectrometry (nano-HPLC-Chip-TOF MS). The structures of human colonic glycans were characterized by analysis on a nano-HPLC-Chip-TOF mass spectrometer as described by Davis et al. (2016) (Molecular & Cellular Proteomics 15(9): 2987-3002) and these results were previously reported in Frese et al. (2017) (mSphere 2(6): e00501-00517). Briefly, the HPLC system used was an Agilent 1200 series unit with a microfluidic chip, which was coupled to an Agilent 6220 series TOF mass spectrometer via chip cube interface. The capillary pump on the chromatography unit loaded the sample onto the 40-nL enrichment column at a flow rate of 4.0 μL/min with a 1-μL injection volume. A nano pump was used for analyte separation on the analytical column, which was 75×43 mm and packed with porous graphitized carbon. Separation was accomplished using a binary gradient of aqueous solvent A (3% acetonitrile (ACN)/water (v/v) in 0.1% formic acid (FA)) and organic solvent B (90% ACN/water (v/v) in 0.1% FA) using a method developed for HMO separation. The sample was introduced into the TOF mass spectrometer via electrospray ionization, which was tuned and calibrated using a dual nebulizer electrospray source with calibrant ions ranging from m/z 118.086 to 2721.895, and data were collected in the positive mode. These untargeted spectra were reanalyzed in the present study.

Glycan data analysis. The untargeted mass spectra were collected (as above) and analyzed using Agilent MassHunter Work station Data Acquisition version B.02.01 on the nanoHPLC-chip/TOF (Frese et al 2017). The “Find Compounds by Molecular Feature” function of the software was used to identify mucin glycan species within 20 ppm of theoretical masses. Compound abundances were expressed as volume in ion counts that corresponded to absolute abundances of the compounds in each sample. 1HexNAc-1NeuAc, 1HexNAc-1Hex-NeuAc, 2HexNAc-1NeuAc, 2HexNAc-1Hex-1Fuc, 2HexNAc-1Hex-1NeuAc, 2HexNAc-1Hex-2Fuc, 3HexNAc-1Hex-1Fuc, 2HexNAc-1Hex-1Fuc-1NeuAc, 2HexNAc-1Hex-1Fuc-2NeuAc, 3HexNAc-1Hex-2NeuAc and 3HexNAc-1Hex-2Fuc-1NeuAc were monitored as they are discriminitively human colonic glycans. Robbe et al. (2004) Rapid Communications in Mass Spectrometry 18(4): 412-420.

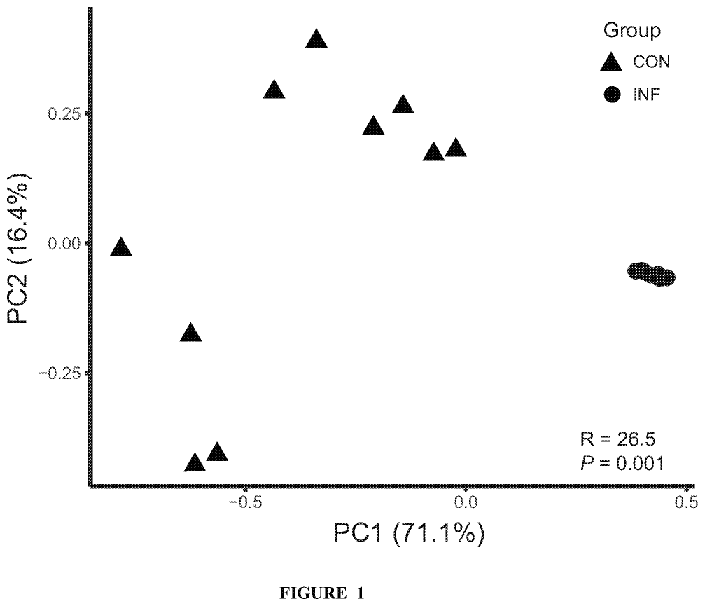

Statistical analysis. Multiple t-tests were carried out with the Holm-Sidak correction in Graph Pad Prism 7 (GraphPad Software, La Jolla, Calif. USA). Pearson and Spearman correlational tests and principle component analyses were performed in R (v 3.4.2). Adonis test was carried out using a weighted UNIFRAC distance matrix (Lozupone et al 2011) in QIIME 1.9.1 (Caporaso et al 2010). Among the 20 samples profiled by nano-HPLC-Chip-TOF MS here, the gut microbiome profiles for 10 infants fed B. infantis EVC001 were significantly different by an adonis test (IV=0.62, P<0.001) from that of the 10 control infants (FIG. 1). The total oligosaccharide (OS) compositions of the samples were determined by the untargeted approach of nano-HPLC-Chip-TOF. The compounds were characterized using previously published libraries. Thomsson et al. (2002) Glycobiology 12(1): 1-14; Robbe et al. (2004) Rapid Communications in Mass Spectrometry 18(4): 412-420; Matamoros et al. (2013) Trends in Microbiology 21(4): 167-173; Karav et al. (2016) Applied and Environ. Micro. 82(12): 3622-3630. Among these compositions, free HMO (Frese et al. (2017) mSphere 2(6): e00501-e00517) and mucin OS were found in the infant fecal glycome. The degradation of human colonic mucin glycans by different gut microbiome profiles was determined as the difference between a B. infantis-dominated microbiome and microbiomes with more abundant mucolytic taxa, such as Bacteroidaceae. As target molecules, 1HexNAc-1NeuAc, 1HexNAc-1Hex-NeuAc, 2HexNAc-1NeuAc, 2HexNAc-1Hex-1Fuc, 2HexNAc-1Hex-1NeuAc, 2HexNAc-1 Hex-2Fuc, 3HexNAc-1Hex-1Fuc, 2HexNAc-1Hex-1Fuc-1NeuAc, 2HexNAc-1Hex-1Fuc-2NeuAc, 3HexNAc-1Hex-2NeuAc and 3HexNAc-1Hex-2Fuc-1NeuAc, were selected as typical human colonic mucin glycans, as shown by Robbe et al. (2004) Rapid Communications in Mass Spectrometry 18(4): 412-420.

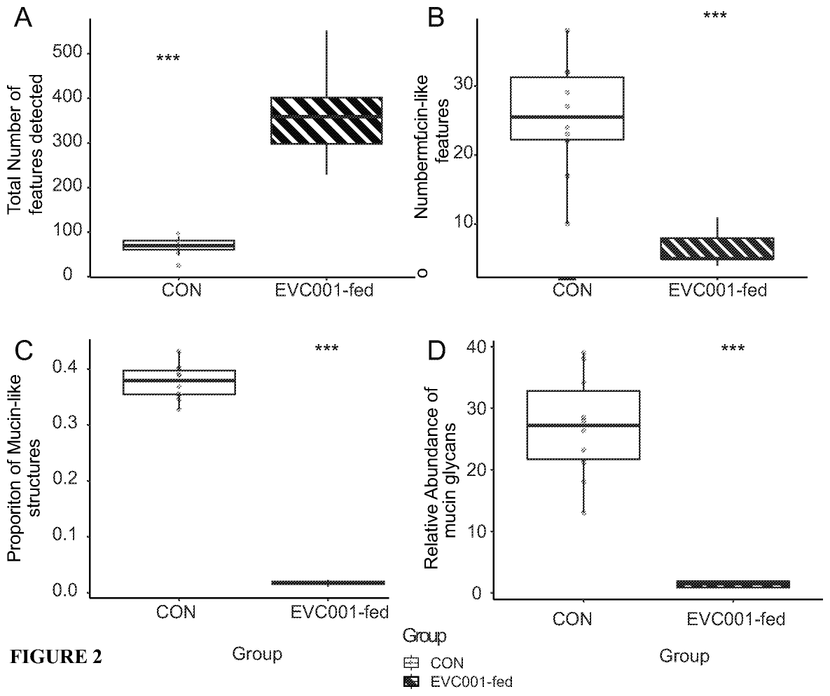

In FIG. 2, the mass spectrometry monitoring these structures showed that the number of total OS structures (including isomers and anomers) in samples from control and EVC001-fed infants ranged from 67.4±19.81 and 360.44±102.52, respectively (P<0.001; FIG. 2A). Although the control samples contained fewer total OS structures, the number of freed human colonic mucin-derived O-glycans of the total OS was significantly higher 25.4 (±8.09), whereas only 6.33 (±2.24) structures were colonic mucin-derived O-glycans in samples from EVC001-fed infants (P<0.001, Wilcoxon test; FIG. 2B). As a proportion, the relative abundance of colonic mucin-derived O-glycans was significantly higher in control samples than in samples from EVC001-fed infants in terms of both the number of structures (37.68%±3.14% and 1.78%±0.385%, respectively; FIG. 2C, P<0.001, Wilcoxon test) and their proportion of the total OS profile (26.98%±8.48% and 1.68±1.12%, respectively; FIG. 2D, P<0.001, Wilcoxon test).

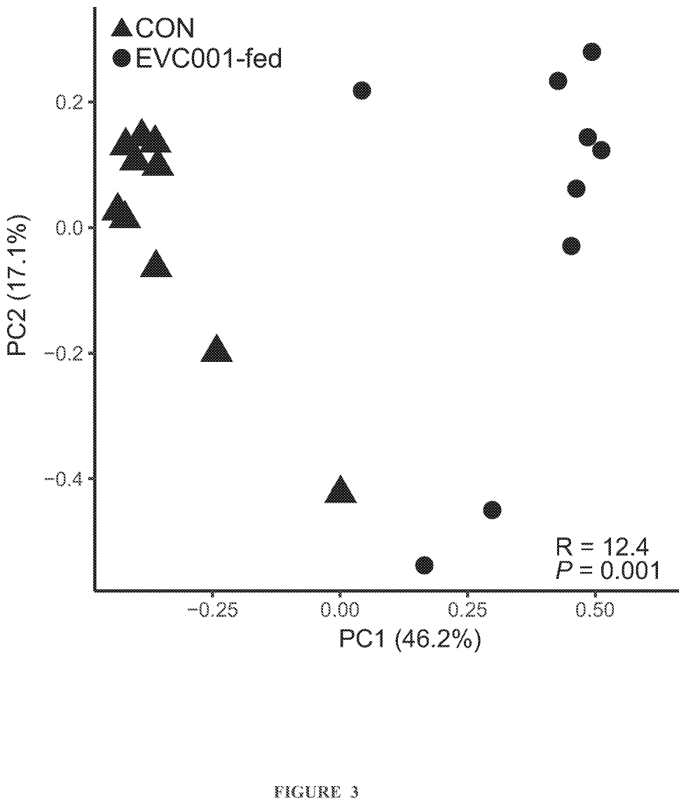

To examine the interactions of the gut microbiome and the mucin OS species, a Pearson correlation was calculated for all taxa and structures in the samples, as well as the total abundance and proportion of OS species. Bifidobacteriaceae abundance was significantly and negatively correlated with all mucin core OS species, whereas Bacteroidaceae was significantly and positively correlated with the abundance of 1_1_0_1, 1_0_0_1, 2_1_1_1, 3_1_2_1, and 2_1_2_0, as well as total percentage and number of mucin core OS species (Figure. 3). Individually, Bacteroidaceae was significantly correlated with the percentage of mucin core OS species with a Spearman's rho of 0.45 (P=0.0393), but Bifidobacteriaceae were more strongly, but negatively, correlated with the percentage of mucin core OS species with a rho of 0.71 (P<0.001). This might indicate that other mucolytic taxa are present in the gut that contributed to or enhance mucin degradation, but that these were also inversely correlated with the abundance of Bifidobacteriaceae. Conversely, this could indicate that Bifidobacteriaceae contribute to the consumption of released mucin OS species. However, this appears unlikely given that B. infantis does not degrade mucin as a sole carbon source. In contrast, members of the Bacteroides allocate a large proportion of their genome to harvesting polysaccharides, including mucin (Xu et al 2003) and were significantly and positively correlated with mucin OS species concentrations release. Many of the genes associated with polysaccharide utilization are highly active on mucin glycoproteins, including the O-glycan cores found in human colonic mucin. Bacteroides can grow on mucin as a sole carbon source and has specific transcriptional responses to incubation with mucin. Marcobal & Sonneburg (2012) Clinical Microbiology and Infection 18(s4): 12-15. In particular, Bacteroides possess enzymes from glycosyl hydrolase family GH 84, GH 85, GH 89, GH 101 and GH 129 that are active on mucin glycoconjugates.

### Example 1C

### Improved Vaccine Response

Infants from Example 1 received vaccines per the standard schedule. Standard schedule is expected to look like this, as per the CDC (other nations have slightly different schedules; however, the repeated doses over the first year of life is common).

Stool samples were collected at 10 and 12 months for vaccine quantification to coincide with an appropriate post-vaccine time point, and to allow the expansion of vaccine-specific antibodies concentrations to plateau after primary, secondary or tertiary doses [Wright et al. (2014) Journal of Infectious Diseases:209 pg 1628-1634; Brown et al. (2012) J Immunol Methods: 386(1-2): 117-12]. Stools are found to demonstrate a significant elevation of vaccine specific antibodies in the treatment group.

Evaluation of EVC001 colonization. As previously described in Frese et al. (2017) (mSphere 2(6): e00501-00517) colonization is measured at baseline and post-feeding by quantitative PCR using specific primers for B. infantis. Colonization is considered when B. infantis abundance is greater than 10⁵, but more preferably greater than 10⁷ or 10⁸ CFU/ug DNA. Colonization may also be described as a significant expansion of the total relative contribution of Bifidobacteriaceae to the infant gut microbiome.

Microbial production of short chain fatty acids (SCFA): SCFA can modulate intestinal inflammation by regulating epithelial barrier function. Microbial short chain fatty acids (SCFA) are extracted by methods previously described and analyzed by gas chromatography HD and found to be elevated in the treatment group

Fecal zonulin concentrations: Zonulin has been described as the main physiological modulator of intercellular tight junction, and increased levels are indicative of increased gut permeability. The concentration of fecal zonulin is determined using commercially-available ELISA kits (Immundiagnostik, Bensheim, Germany), as previously described and found to be significantly reduced in the treatment group

Fecal TNF expression: TNF is a cytokine that plays a key role in mucosal inflammation and is readily detectable at the protein level in children with intestinal pathology and found to be significantly reduced in the treatment group

Urine levels of fatty acid binding proteins (FABPs) and glutathione S-transferase (α-GST): FABPs are small, water-soluble cytosolic proteins that are released into the circulation following loss of enterocyte membrane integrity and are thus markers of gastrointestinal permeability, which has been shown to increase in states of systemic inflammation. Glutathione S-transferases (α-GST) are enzymes present predominantly in liver, kidney and intestinal epithelial cells that are responsible for detoxification of intracellular toxins through conjugation to glutathione. In states of increased intestinal inflammation and permeability, plasma levels of (α-GST) are a peripheral marker of intestinal epithelial cell injury. Levels of these biomarkers are assessed using the MILLIPLEX MAP luminex assay (BioRad) following their standard protocol and found to be significantly reduced in the treatment group

Expression of inflammatory markers including Toll like receptor (TLR) 2 and TLR4, COX-2 and TNF in intestinal epithelial cells: Previous data has implemented TLR2 in controlling mucosal inflammation by regulating intestinal epithelial barrier function. Further, a significant TLR4-dependent increase in Cox-2 expression has been shown in intestinal epithelial cells post exposure to lipopolysaccharide. TNF-α, a cytokine that plays a key role in mucosal inflammation, is readily detectable at the expression and protein levels in children who have various intestinal pathologies. The expression of TLR2, TLR4, TNF and COX-2 are determined using qPCR in sloughed off epithelial cells, as shown previously and found to be significantly reduced in the treatment group

Fecal Microscopy. All samples were fixed by adding equal amount of 5% glutaraldehyde in 0.2 M cacodylate buffer (final concentration 2.5% glutaraldehyde in 0.1 M cacodylate buffer) for 1-2 h before being processed for Gram-stained light microscopy and scanning electron microscopy (SEM). For light microscopy, conventional Gram-stained samples on slides were imaged under an EVOS Auto-FL system using a 60× lens and 2.7× optical zoom, under the same setting of the color camera. Five random fields of images were collected from each sample slide. For SEM, since the samples were previously frozen before fixation, the osmium post-fixation, and critical-point dry procedures were not performed. The fixed samples were dehydrated through an ethanol series and placed on membrane filters. The samples were mounted onto the SEM stubs, aired overnight, and then vacuum-oven dried at 50° C. for >2 h before sputter-coating with a thin layer of chromium using Denton Desk V sputter. Images were collected under various magnifications to capture bacterial morphology using a Hitachi S4700 field-emission SEM. Microscopy confirmed the high levels of Bifidobacterium in samples from the treatment group.

Bacterial DNA Methods. The relative abundance of the stool bacteria at the phylum, class, order, family, and genus level was characterized by performing a sequence analysis of the V4 segment of the 16S rRNA gene using QIIME v1.9.1.

Fecal Calprotectin. The level of fecal calprotectin was quantified using IDK Calprotectin ELISA kit (Immundiagnostik AG, Germany) in accordance with the manufacturer's instructions. Absorbance was read at a wavelength of 450 nm using a Synergy HT Multi-Detection Microtiter Plate Reader (BioTek, USA). The samples were plated in duplicate and the assay was performed twice.

Multiplexed Immunoassays. Interleukin (IL)-1β, IL-2, IL-5, IL-6, IL-8, IL-10, IL-22, interferon (IFN) γ, and tumor necrosis factor (TNF) α were quantified from 80 mg of stool diluted 1:10 in Meso Scale Discovery (MSD; Rockville, Md.) diluent using the U-PLEX Inflammation Panel 1 (human) Kit according to the manufacturer's instructions. Standards and samples were measured in duplicate and blank values were subtracted from all readings. The plate was then read on a Sector Imager 2400 MSD Discovery Workbench analysis software. Statistical Analysis. Demographic differences between Control and EVC001-fed infants was analyzed using Fisher's Exact test for categorical data and Wilcoxon rank sum (Mann-Whitney U) test for continuous data. Table 7 shows data eliminated from analysis or visualization. The relationship between fecal calprotectin concentration and % Bifidobacteriaceae was quantified using Spearman's Rho correlation, and the differences in calprotectin concentration between high (>25%) and low (<25%) Bifidobacteriaceae abundance was assessed using the Wilcoxon rank sum test. One subject with abnormally high fecal calprotectin concentration (greater than 3 standard deviations above the mean of both treatment and control data) was considered an extreme outlier and removed from the aforementioned calprotectin analyses. Wilcoxon rank sum tests were performed to assess relative abundance for each bacterial taxa and cytokine concentration differences. For radar plots, medians were adjusted to log scale, then normalized within each cytokine group from 0 to 1. Differences overtime within each cytokine group were evaluated using the Wilcoxon rank sum test. P-values were adjusted using the Bonferroni-Holms method and considered statistically significant if P<0.05. Statistical analyses were applied to determine the significance of global cytokine profile as determined by computing a Bray-Curtis distance metric translated into a principal coordinate analysis and visualized with EMPeror. Global cytokine profile differences by group status were then determined using a permutational multivariate analysis of variance (PERMANOVA), and significant P-values were determined using 999 Monte Carlo permutations. To assess the relationship between the global cytokine profile and the microbiome, we used a Procrustes analysis. The taxonomic operational taxonomic unit (OTU) table at the family level was computed using QIIME, and the cytokine table was used to generate a distance matrix for each, using a weighted UniFrac for 16S and Bray-Curtis for the cytokine. We performed a principal coordinate analysis separately on the two matrices and used a Procrustes analysis as implemented in QIIME to rotate, translate, and scale the matrices. The resulting transformed matrices were plotted using EMPeror. P-values for the Procrustes analysis were generated using Monte Carlo simulations (n=999). Raw correlation statistics were assigned the likelihood of these associations to be true positive associations by computing P-values via a Fisher's Z transform to normalize the distribution of the correlation scores.

TABLE 7
Data eliminated from analysis or visualization

| Day | Treatment | Cytokine | Concentration | Data Action | Reason |
|---|---|---|---|---|---|
| 6 (Baseline) | Control | IL2 | 2247.74 | not shown in boxplot | plot scale reduced to 0-750 to better |
| | | | | | showcase data |
| 6 (Baseline) | Control | IL5 | 399.91 | not shown in boxplot | plot scale reduced to 0-50 to better |
| | | | | | showcase data |
| 40 | Control | IL6 | 45.66 | not shown in boxplot | plot scale reduced to 0-35 to better |
| | | | | | showcase data |
| 40 | Control | IL8 | 43779.38 | excluded from analysis | extreme outlier (possible assay error) |
| 6 (Baseline) | Control | IL10 | 236.29 | excluded from analysis | extreme outlier (possible assay error) |
| 40 | Control | IL22 | 167.74 | not shown in boxplot | plot scale reduced to 0-60 to better |
| | | | | | showcase data |
| 6 (Baseline) | Control | TNFα | 606.48 | not shown in boxplot | plot scale reduced to 0-100 to better |
| | | | | | showcase data |
| 40 | Control | TNFα | 301.57 | not shown in boxplot | plot scale reduced to 0-100 to better |
| | | | | | showcase data |
| 40 | Control | IL-1β | 23899.20 | not shown in boxplot | plot scale reduced to 0-10000 to better |
| | | | | | showcase data |
| 40 | Control | IL-1β | 35845.38 | not shown in boxplot | plot scale reduced to 0-10000 to better |
| | | | | | showcase data |
| 60 | Control | IFNγ | 1476.40 | not shown in boxplot | plot scale reduced to 0-1000 to better |
| | | | | | showcase data |

### Results

Demographics and fecal analyses of the infant participants. To investigate the effect of the intestinal microbiome on the host immune response, we used fecal samples from 20 Controls and 20 EVC001—-fed at Days 6 (baseline), 40 and 60 days postnatal. Subjects in the EVC001 and Control groups did not differ with respect to the parameters selected for randomization; however, the Control infants were born to mothers who were significantly more likely to be first time mothers (P<0.01) and significantly younger than the mothers whose infants were randomized to receive B. infantis EVC001 (P<0.01). There were no significant differences between the groups with respect to hours in labor, birth mode, antibiotic use during labor, gestational age, sex, birth or discharge weight, birth length, maternal BMI or gestational weight gain, or maternal GBS diagnosis.

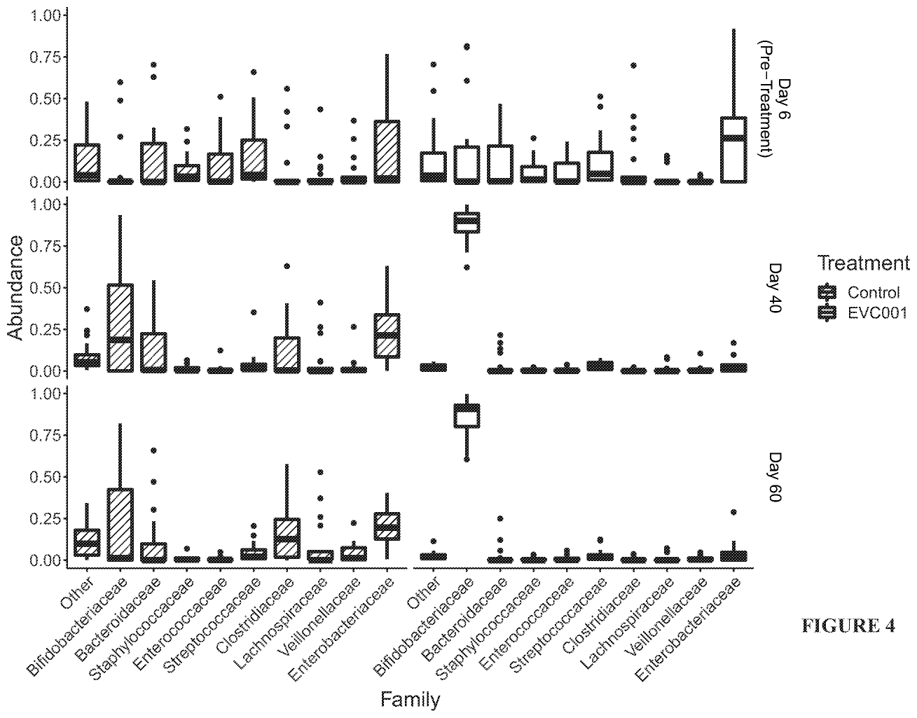

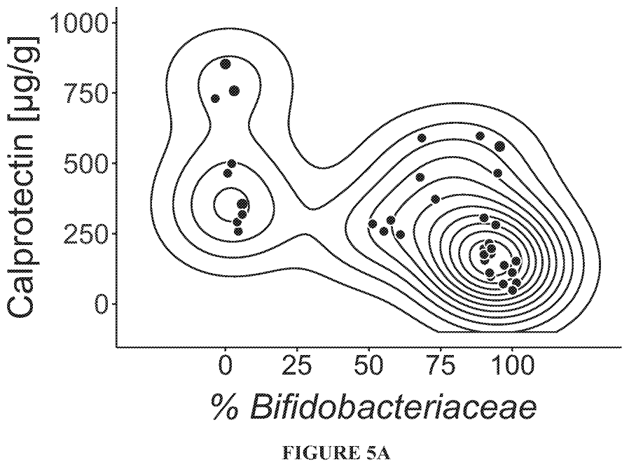

Infants fed B. infantis EVC001 had significant increases in the abundance of Bifidobacteriaceae in the infant gut microbiome. We first evaluated the microbiome profile from the two groups on Day 6 (baseline), Day 40, and Day 60 postnatal (Table 8). The infants included in this prospective study exhibited no statistical differences between the two groups in the four major representative taxa (Bifidobacteriaceae, Bacteroidaceae, Bifidobacteriaceae, Clostridiaceae, and Enterobacteriaceae) that can be identified on Day 6 postnatal, prior to the start of supplementation on Day 7 (FIG. 4). At Day 40, there was a significantly higher abundance in Bifidobacteriaceae in the B. infantis EVC001-fed group compared to the Controls (P<0.0001) (FIG. 4). Conversely, the abundance of Bacteroidaceae and Clostridiaceae taxa were significantly lower, and Enterobacteriaceae was highly significantly lower in infants who were treated with EVC001 (P<0.05, P<0.05, and P<0.0001, respectively) (FIG. 4). Similarly, on Day 60 postnatal (32 days after last feed with EVC001), the microbiome composition of the infants fed EVC001 displayed higher abundance of Bifidobacteriaceae, as well as a lower abundance of Bacteroidaceae and Clostridiaceae compared to the Control infants (all P<0.0001) (FIG. 4).

TABLE 8
Microbiome profile of infants that received B. infant is EVC001

| | Day 6 (Baseline) | | | Day 40 | | | Day 60 | | |
|---|---|---|---|---|---|---|---|---|---|
| | % Relative Abundance | | | % Relative Abundance | | | % Relative Abundance | | |
| Family | Control | EVC001 | P value | Control | EVC001 | P value | Control | EVC001 | P value |
| Bifidobacteriaceae | 0.0698 | 0.1472 | 0.686 | 0.2939 | 0.8712 | 1.20E−06 | 0.2242 | 0.8532 | 1.10E−06 |
| Bacteroidaceae | 0.1275 | 0.1040 | 0.499 | 0.1297 | 0.0275 | 0.02331 | 0.1011 | 0.0202 | 0.0716 |
| Staphylococcaceae | 0.0722 | 0.0543 | 0.54 | 0.0130 | 0.0036 | 0.1956 | 0.0100 | 0.0039 | 0.0118 |
| Enterococcaceae | 0.0962 | 0.0539 | 0.884 | 0.0079 | 0.0045 | 0.54546 | 0.0066 | 0.0082 | 0.9019 |
| Streptococcaceae | 0.1534 | 0.1169 | 0.686 | 0.0386 | 0.0291 | 0.56182 | 0.04818 | 0.0277 | 0.2952 |
| Clostridiaceae | 0.0722 | 0.0874 | 0.424 | 0.1141 | 0.0016 | 0.02259 | 0.1635 | 0.0040 | 3.60E−05 |
| Lachnospiraceae | 0.0403 | 0.0197 | 0.966 | 0.0503 | 0.0072 | 0.10413 | 0.0811 | 0.0081 | 0.1889 |
| Veillonellaceae | 0.0467 | 0.0065 | 0.033 | 0.0219 | 0.0077 | 0.1235 | 0.0453 | 0.0076 | 0.892 |
| Enterobacteriaceae | 0.1929 | 0.2743 | 0.593 | 0.2393 | 0.0260 | 9.60E−06 | 0.1940 | 0.0427 | 7.60E−05 |
| Other | 0.1286 | 0.1357 | 0.969 | 0.0912 | 0.0215 | 0.00048 | 0.1260 | 0.02443 | 0.0024 |

Light and scanning electron microscopy was used in three fecal samples from Day 40 from each of the Control and EVC001-fed groups, respectively. Gram staining showed fecal smears of Controls contained predominantly Gram-negative bacteria, while samples from EVC001-fed infants overwhelmingly contained Gram-positive bacteria. Multiple fields of view of the fecal samples from the Control group identified several distinct bacterial morphologies, whereas samples from the EVC001-fed infants exhibited a uniform morphology of rod-shaped bacteria that are infrequently longitudinally split, which is in agreement with our molecular observations.

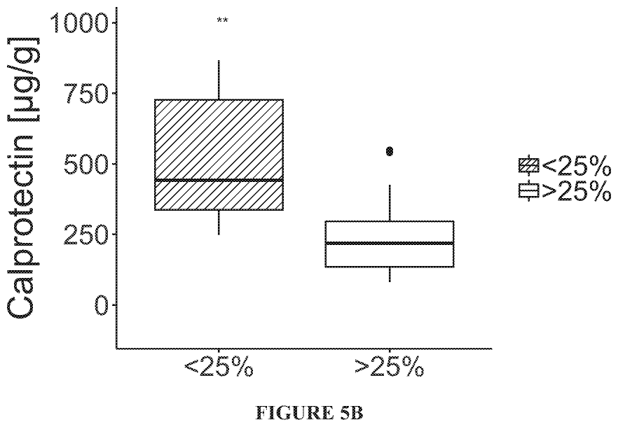

Fecal calprotectin levels are directly correlated with the abundance of Bifidobacteriaceae. Dysbiosis, including low abundance of Bifidobacteriaceae in the infant gut, has been associated with increased inflammation. To determine whether Bifidobacterium abundance in the gut was associated with enteric inflammation in our cohort, concentrations of fecal calprotectin, a marker of intestinal inflammation, were compared to the abundance of bacterial taxa using a Spearman's correlation analysis from stools collected at Day 40 postnatal. Data observed from Day 40 showed a significant correlation between Bifidobacterium abundance and lower fecal calprotectin levels (rs=−0.72, P<0.0001; FIG. 5a). These data also provide a clear bimodal distribution in which Bifidobacteriaceae abundance 25% is considered low Bifidobacteriaceae and >25% represents high Bifidobacteriaceae (FIG. 5a). Using a 25% abundance cut-off, samples that contained low Bifidobacteriaceae showed significantly increased enteric inflammation (as measured by fecal calprotectin) compared to samples that contained high levels of Bifidobacteriaceae (P<0.01; FIG. 5b).

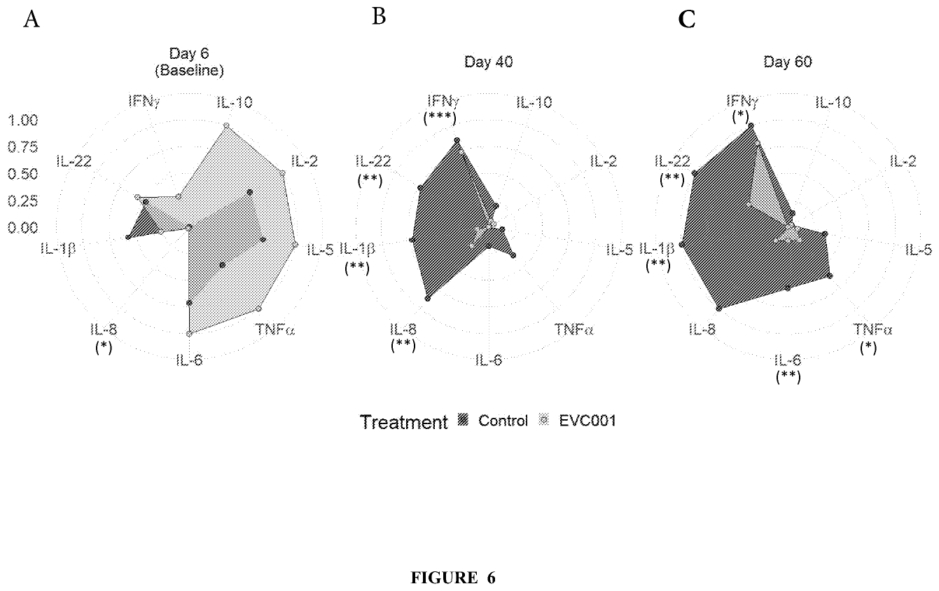

Colonization with B. infantis EVC001 is associated with decreased fecal pro-inflammatory cytokine expression. The cytokine profile on Day 6, Day 40, and Day 60 postnatal was evaluated. At baseline there was a significantly higher concentration of IL-10 production in the Control compared with EVC001 infants (P<0.05; FIG. 6a); however, no other statistically significant differences could be identified between the two groups at baseline. By Day 40 postnatal, we observed a significant modulation of the fecal cytokine profiles in the EVC001-fed infants, compared to the infants in the Control group. Specifically, concentrations of IL-8, IL-22, IL-1β were significantly lower in EVC001 infants compared to the fecal samples from the Controls (all P<0.01; FIG. 6b) and IFNγ (P<0.001; FIG. 6b), respectively. This trend continued at Day 60, in which time the Controls produced significantly higher levels of IL-6, IL-22, TNFα, IL-1β, and IFNγ compared to infants who were colonized with B. infantis EVC001 (P<0.01, P<0.01, P<0.05, P<0.01, and P<0.05 respectively, FIG. 6c). Taken together, these data show major global cytokine differences between exclusively breastfed infants fed EVC001 compared to those that were not over the first 60 days of life (Table 9).

TABLE 9
Significance of fecal cytokine signature of infants that received B. infantis EVC001

| | Day 6 (Baseline) | | | | | Day 40 | | | |
|---|---|---|---|---|---|---|---|---|---|
| | Median (SD) pg/mg | | | | | Median (SD) pg/mg | | | |
| Cytokine | Control | | EVC001 | | P value | Control | | EVC001 | |
| IL-2 | 17.93 | (1116.6) | 48.13 | (39.48) | 1 | 2.87 | (7.8) | 3.36 | (4.41) |
| IL-5 | 7.95 | (138.82) | 16.53 | (10.87) | 1 | 2.02 | (2.1) | 1.48 | (1.3) |
| IL-6 | 3.98 | (9 07) | 9.29 | (8.43) | 1 | 0.83 | (10.71) | 0.48 | (1) |
| IL-8 | 46.49 | (2023.91) | 48.35 | (406.67) | 1 | 354.84 | (4383.55) | 81.67 | (87.87) |
| IL-10 | NA | (NA) | 8.76 | (5.56) | NA | 0.85 | (2.26) | 0.55 | (0.4) |
| IL-22 | 3.77 | (2.68) | 4.22 | (2.17) | 1 | 5.29 | (36.85) | 2.07 | (2.07) |
| TNFα | 8.04 | (154.19) | 26.71 | (20.47) | 0.16 | 6.07 | (67.16) | 2.7 | (2.62) |
| IL-1β | 132.95 | (1431.15) | 38.65 | (168.63) | 0.026 | 237.02 | (9241) | 20.27 | (74.56) |
| IFNγ | 0.02 | (0.34) | 0.29 | (2.14) | 0.1 | 34.26 | (176.69) | 12.99 | (18.16) |

| | | Day 60 | | | | |
|---|---|---|---|---|---|---|
| | Day 40 | Median (SD) pg/mg | | | | |
| Cytokine | P value | Control | | EVC001 | | P value |
| IL-2 | 1 | 3.23 | (12.85) | 3.16 | (2.26) | 1 |
| IL-5 | 0.84 | 3.47 | (7 06) | 1.85 | (1.63) | 0.49 |
| IL-6 | 0.63 | 2.65 | (9.22) | 0.71 | (1.12) | 0.0054 |
| IL-8 | 0.0054 | 469.98 | (3944.92) | 70.13 | (736.71) | 0.069 |
| IL-10 | 0.82 | 0.68 | (0.6) | 0.46 | (0.76) | 0.82 |
| IL-22 | 0.0023 | 7.46 | (17.35) | 3.54 | (3.43) | 0.0098 |
| TNFα | 0.086 | 10.82 | (14.67) | 3.97 | (2.67) | 0.012 |
| IL-1β | 0.0037 | 724.61 | (2773.7) | 13.22 | (193.24) | 0.0038 |
| IFNγ | 0.00073 | 119.1 | (380.22) | 26.74 | (43.16) | 0.013 |

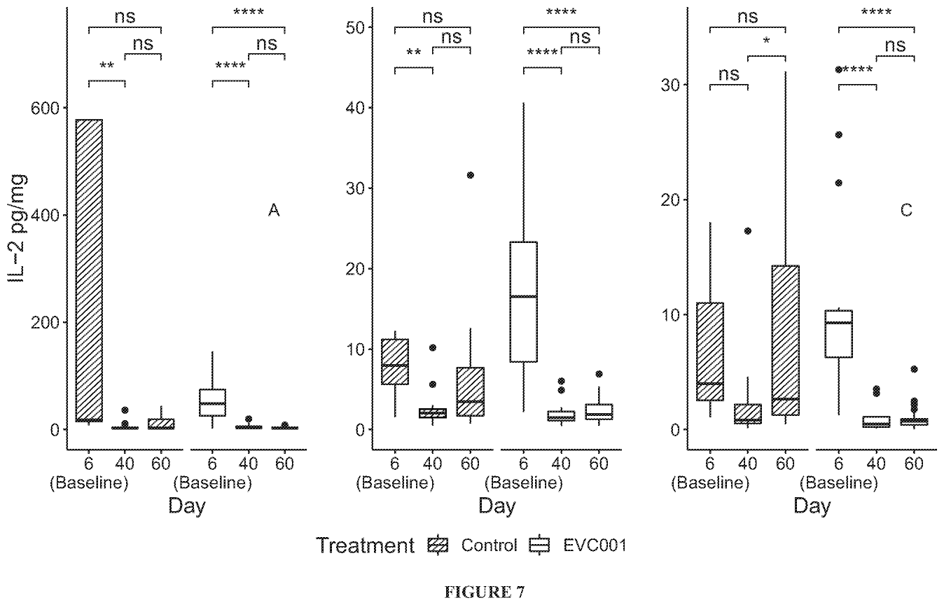

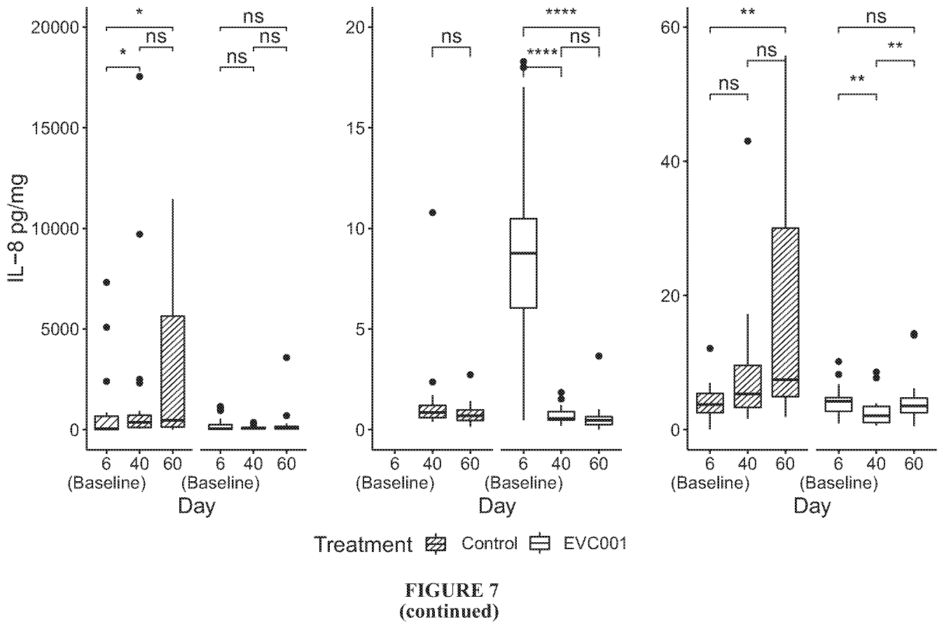

Control infants showed a significant reduction in IL-2 and IL-5 from Day 6 to Day 40 (P<0.05 and P<0.01; FIG. 7a, b, and i); however, controls showed significantly higher levels of IFNγ from Day 6 to Day 40 (P<0.0001). Further, there was no significant difference in cytokine levels in Control infants from Day 40 to Day 60 (FIG. 7a-i); yet, IL-22 and IFNγ were significantly higher in Day 60 versus Day 6 fecal samples (P<0.01, P<0.0001, respectively; FIGS. 7f and i).

Conversely, infants fed EVC001 produced significantly lower levels of IL-2, IL-5, IL-6, IL-10, IL-22, TNFα; yet significantly increased IFNγ (all P<0.0001; FIG. 7a-f) at Day 40 compared to Day 6. Further, IL-22 at Day 60 was significantly higher than Day 40 (P<0.05; FIG. 7f). Fecal levels of IL-2, IL-5, IL-6, IL-10, and TNFα at Day 60 were significantly lower compared to Day 6 (all P<0.0001; FIG. 7a-c, e, g), while IFNγ levels were significantly higher at Day 60 compared to Day 6 (P<0.0001; FIG. 7i).

These results showed chief differences in cytokine distribution during the first 60 days of life between EVC001-fed and Control infants. Most notably, fecal cytokine levels were significantly lower in infants that received EVC001 and remained low during the first 60 days postnatal, while the Control infants had varying levels dependent on the cytokine, but overall cytokine levels increased postnatally from Day 6 to Day 60 (Table 10).

TABLE 10
Significance of fecal cytokine concentrations change postnatally

| Day 6 (Baseline) to Day 40 | | |
|---|---|---|
| | Control | EVC001 |

| | Median (SD) pg/mg | | | | | Median (SD) pg/mg | | | | |
|---|---|---|---|---|---|---|---|---|---|---|
| Cytokine | Day 6 (Baseline) | | Day 40 | | P value | Day 6 (Baseline) | | Day 40 | | P value |
| IL-2 | 17.93 | (1116.6) | 2.87 | (7.8) | 0.03 | 48.13 | (39.48) | 3.36 | (4.41) | 1.40E−05 |
| IL-5 | 7.95 | (138.82) | 2.02 | (2.1) | 0.0084 | 16.53 | (10.87) | 1.48 | (1.3) | 1.30E−06 |
| IL-6 | 3.98 | (9.07) | 0.83 | (10.71) | 0.31 | 9.29 | (8.43) | 0.48 | (1) | 1.50E−05 |
| IL-8 | 46.49 | (2023.91) | 354.84 | (4383.55) | 0.17 | 48.35 | (406.67) | 81.67 | (87.87) | 1 |
| IL-10 | NA | (NA) | 0.85 | (2.26) | NA | 8.76 | (5.56) | 0.55 | (0.4) | 6.70E−06 |
| IL-22 | 3.77 | (2.68) | 5.29 | (36.85) | 0.17 | 4.22 | (2.17) | 2.07 | (2.07) | 0.01 |
| TNFα | 8.04 | (154.19) | 6.07 | (67.16) | 1 | 26.71 | (20.47) | 2.7 | (2.62) | 1.80E−05 |
| IL-1β | 132.95 | (1431.15) | 237.02 | (9241) | 1 | 38.65 | (168.63) | 20.27 | (74.56) | 0.58 |
| IFNγ | 0.02 | (0.34) | 34.26 | (176.69) | 9.30E−06 | 0.29 | (2.14) | 12.99 | (18.16) | 0.0001 |

Day 6 (Baseline) to Day 60

| | Control | | | | | EVC001 | | | | |
|---|---|---|---|---|---|---|---|---|---|---|
| | Median (SD) pg/mg | | | | | Median (SD) pg/mg | | | | |
| Cytokine | Day 6 (Baseline) | | Day 60 | | P value | Day 6 (Baseline) | | Day 60 | | P value |
| IL-2 | 17.93 | (1116.6) | 3.23 | (12.85) | 0.4 | 48.13 | (39.48) | 3.16 | (2.26) | 1.10E−05 |
| IL-5 | 7.95 | (138.82) | 3.47 | (7.06) | 0.21 | 16.53 | (10.87) | 1.85 | (1.63) | 2.40E−06 |
| IL-6 | 3.98 | (9.07) | 2.65 | (9.22) | 0.89 | 9.29 | (8.43) | 0.71 | (1.12) | 3.20E−06 |
| IL-8 | 46.49 | (2023.91) | 469.98 | (3944.92) | 0.17 | 48.35 | (406.67) | 70.13 | (736.71) | 1 |
| IL-10 | NA | (NA) | 0.68 | (0.6) | NA | 8.76 | (5.56) | 0.46 | (0.76) | 6.30E−06 |
| IL-22 | 3.77 | (2.68) | 7.46 | (17.35) | 0.0079 | 4.22 | (2.17) | 3.54 | (3.43) | 0.37 |
| TNFα | 8.04 | (154.19) | 10.82 | (14.67) | 1 | 26.71 | (20.47) | 3.97 | (2.67) | 6.90E−05 |
| IL-1β | 132.95 | (1431.15) | 724.61 | (2773.7) | 1 | 38.65 | (168.63) | 13.22 | (193.24) | 0.058 |
| IFNγ | 0.02 | (0.34) | 119.1 | (380.22) | 8.30E−06 | 0.29 | (2.14) | 26.74 | (43.16) | 5.00E−05 |

Day 40 to Day 60

| | Control | | | | | EVC001 | | | | |
|---|---|---|---|---|---|---|---|---|---|---|
| | Median (SD) pg/mg | | | | | Median (SD) pg/mg | | | | |
| Cytokine | Day 40 | | Day 60 | | P value | Day 40 | | Day 60 | | P value |
| IL-2 | 2.87 | (7.8) | 3.23 | (12.85) | 0.81 | 3.36 | (4.41) | 3.16 | (2.26) | 0.81 |
| IL-5 | 2.02 | (2.1) | 3.47 | (7.06) | 0.21 | 1.48 | (1.3) | 1.85 | (1.63) | 0.33 |
| IL-6 | 0.83 | (10.71) | 2.65 | (9.22) | 0.072 | 0.48 | (1) | 0.71 | (1.12) | 0.89 |
| IL-8 | 354.84 | (4383.55) | 469.98 | (3944.92) | 1 | 81.67 | (87.87) | 70.13 | (736.71) | 1 |
| IL-10 | 0.85 | (2.26) | 0.68 | (0.6) | 0.25 | 0.55 | (0.4) | 0.46 | (0.76) | 0.13 |
| IL-22 | 5.29 | (36.85) | 7.46 | (17.35) | 0.27 | 2.07 | (2.07) | 3.54 | (3.43) | 0.038 |
| TNFα | 6.07 | (67.16) | 10.82 | (14.67) | 1 | 2.7 | (2.62) | 3.97 | (2.67) | 0.36 |
| IL-1β | 237.02 | (9241) | 724.61 | (2773.7) | 1 | 20.27 | (74.56) | 13.22 | (193.24) | 0.47 |
| IFNγ | 34.26 | (176.69) | 119.1 | (380.22) | 0.062 | 12.99 | (18.16) | 26.74 | (43.16) | 0.054 |

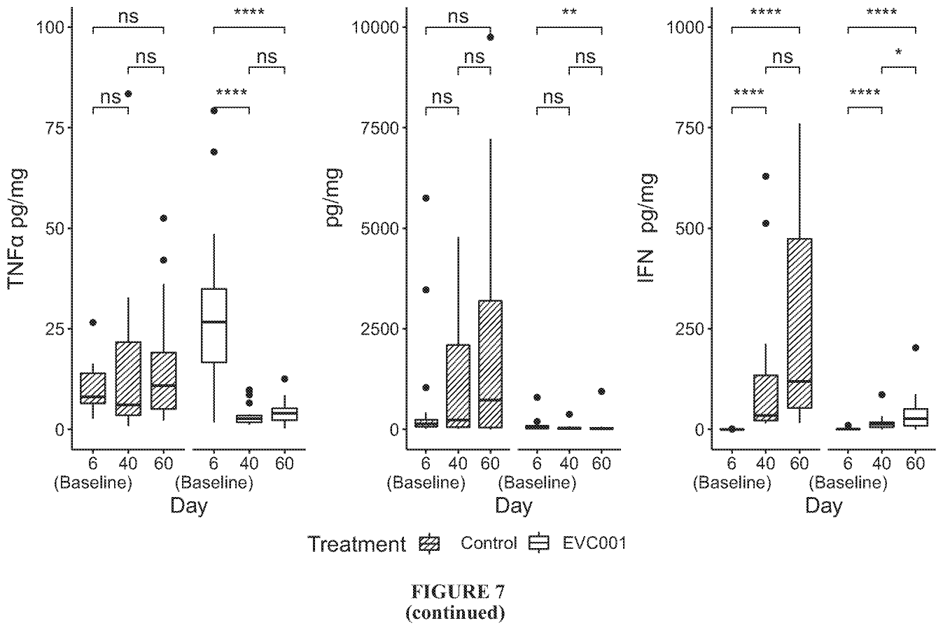

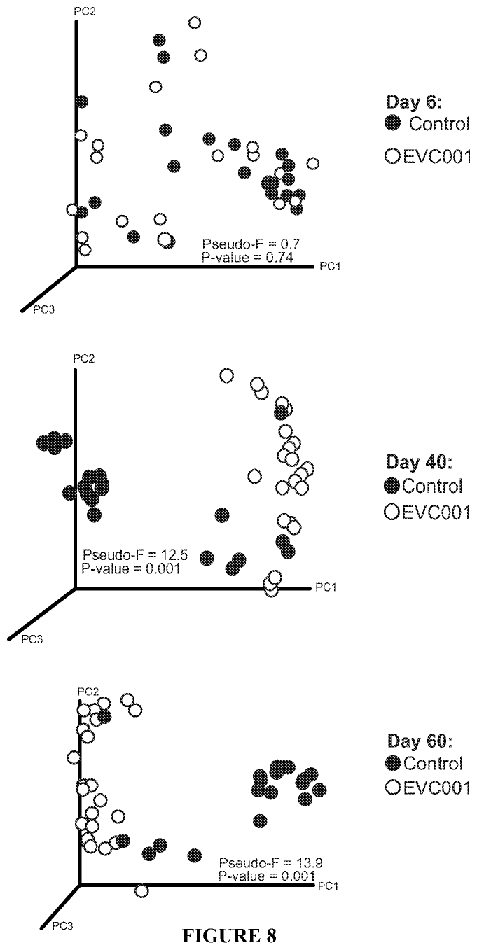

Colonization with B. infantis EVC001 influences cytokine profiles. To identify the main driver of the measured fecal cytokines, we used a principal component analysis (PCA) as a dimension-reduction technique using all parameters of clinical data stated above, proinflammatory cytokine concentrations, and group. With the addition of the clinical data, the cytokine profile composition did not differ among the infants on Day 6 prior to receiving EVC001, as shown by the PCA for the Day 6 fecal samples (FIG. 8a); however, by Day 40 postnatal, distinct clustering was evident between the EVC001-fed and the Control group (P=0.001, Pseudo-F=12.5; FIG. 8b). Such separation remained evident on Day 60, with more pronounced clustering compared to the earlier time points (P=0.001, Pseudo-F=13.9; FIG. 8c). These observations confirmed time and colonization with B. infantis EVC001 are the primary factors influencing differences in fecal cytokines.

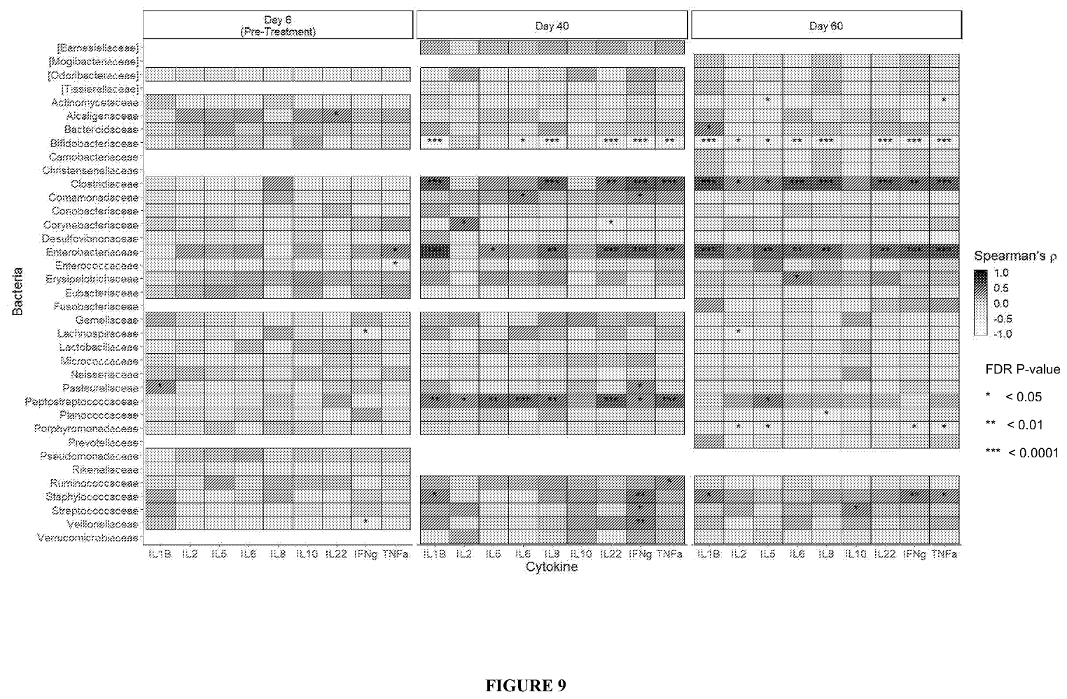

Significant correlations exist between gut microbial abundance and intestinal inflammatory cytokine responses. We performed pairwise correlation tests between the microbial taxonomic composition and specific cytokine concentration detected in the feces of exclusively breastfed infants on Day 6 (Baseline) as well as Day 40 and Day 60 postnatal (Spearman correlation with Benjamini-Hochberg FDR correction α<0.02). A total of four taxa were discovered to be significantly correlated with specific proinflammatory cytokines, including, Clostridiaceae, Enterobacteriaceae, Peptostreptococcaceae, and Staphylococcacea. Specifically, Clostridiaceae was significantly correlated with the production of IL-1β, IL-8, IFNγ, and TNFα at Day 40, and IL-1β, IL-6, IL-8, IL-22, IFNγ, and TNFα at Day 60 postnatal. Enterobacteriaceae was significantly correlated with increased levels of IL-1β, IL-8, IL-22, IFNγ, and TNFα on Day 40, and IL-1β, IL-6, IL-22, IFNγ, and TNFα at Day 60 postnatal, Peptostreptococcaceae significantly correlated with IL-22 and TNFα on Day 40, and Staphylococcaceae correlated with increased IFNγ concentration on Day 40. Furthermore, five proinflammatory cytokines (IL-1β, IL-8, IL-22, IFNγ, and TNFα) were discovered to be negatively correlated with Bifidobacterium at Day 40 postnatal, as well as six proinflammatory cytokines (IL-1β, IL-6, IL-8, IL-22, IFNγ, and TNFα) negatively correlated on Day 60 postnatal (FIG. 9)).

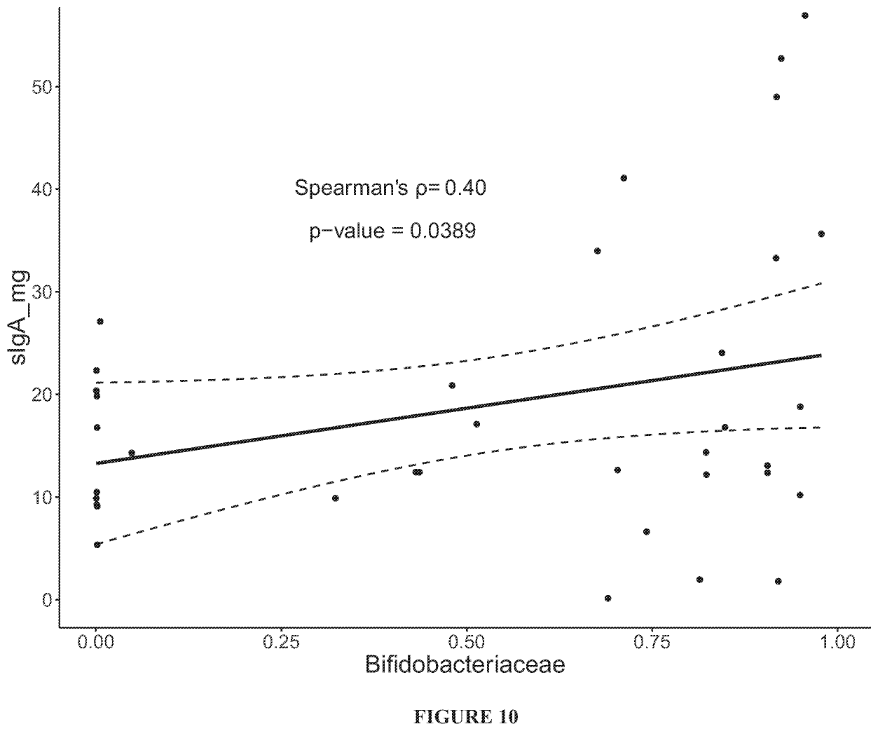

Evaluation of Secretory IgA in the infant stool. Fecal samples from Example 1 were examined for sIgA by a sandwich enzyme immunoassay for in vitro quantitative measurement (RedBlot, CA; https://www.reddotbiotech.ca/files/manuals/5c63251c-5b44-4771-b920-55e8d8b0b5a9.pdf). The results were then correlated with Bifidobacteriaceae family relative abundance by 16s DNA sequencing. The results in FIG. 10 show that sIgA increase with increasing Bifidobacteriaceae Spearman's p=0.40, p-value=0.0389. This is used as an example, but not meant to limit one skilled in the art of using different compositions outlined here to increase Bifidobacteriaceae, or different study designs to evaluate the benefit of increasing sIgA.

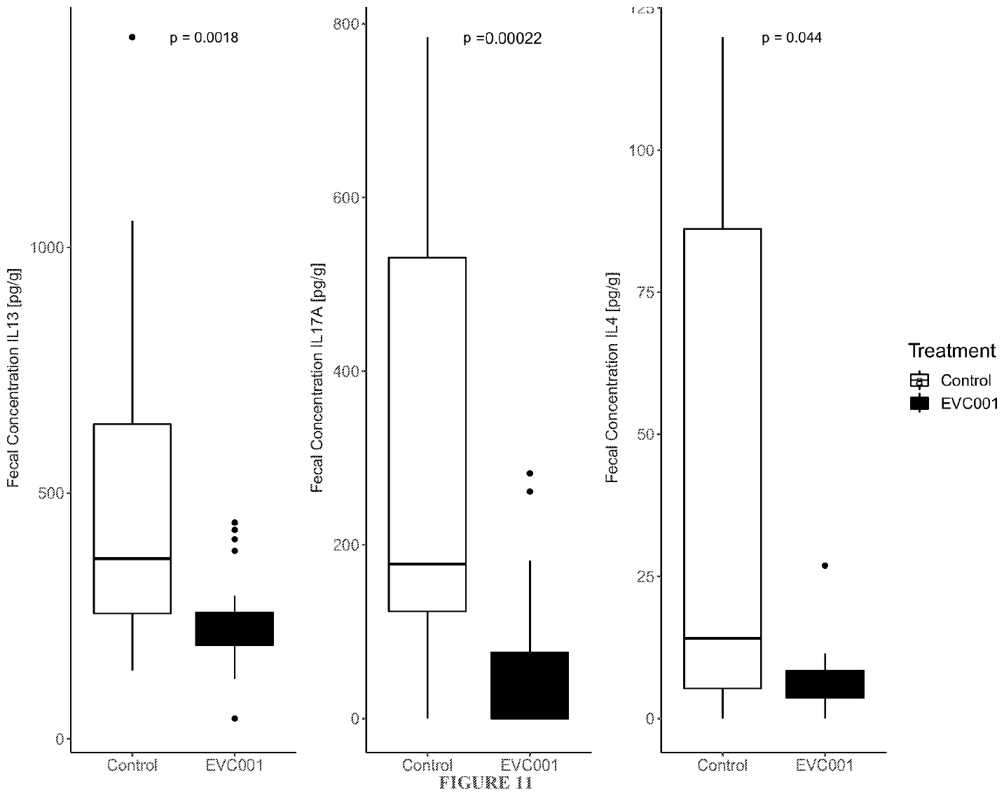

Evaluation of IL-17, IL-4 and IL-13). The difference in IL-17 between control and EVC001 treated infants may be useful as a measure of dysbiosis (FIG. 11).

Evaluation of stool for antigen specific responses in infants colonized with B. infantis EVC001. Biophysical antibody profiling assays are performed to assess the Fv and Fc characteristics of vaccine-elicited antibodies raised in infants with and without B. infantis colonization. First, fluorescently-coded magnetic microspheres are functionalized with antigens listed in Table 11. Other antigens may be included or exchanged with those listed in Table 11 as appropriate. Presence and phenotype of antibodies in test samples are assessed by staining of bead-bound antibodies with PE-conjugated detection reagents (Anti-IgG, Anti-IgA, and potentially Anti-IgE) and detection via flow cytometry. For the assay, 100 μL each of prepared stool samples from 34 subjects (2 timepoints, up to 62 samples total) that received either received EVC001 or are part of the control group.

TABLE 11
vaccine testing protocol

| Measurement | Antigen Name | | |
|---|---|---|---|
| Poliovirus | PV1 | PV2 | PV3 |
| Tdap | Tetanus Toxin | Diphtheria Toxin | Pertactin |
| Hepatitis B | Hepatitis B Surface | | |
| | Antigen | | |
| Other | | | |
| Rotavirus | RotaTeq | VP6 | |
| Total Antibody | IgG | IgA | |

Infants fed B. infantis EVC001 and sustained or persistent colonized by B. infantis for at least the first 100 days of life may be observed to show a stronger vaccine response than infants without B. infantis colonization.

### Example 2

### Expansion of Murine TREG Populations

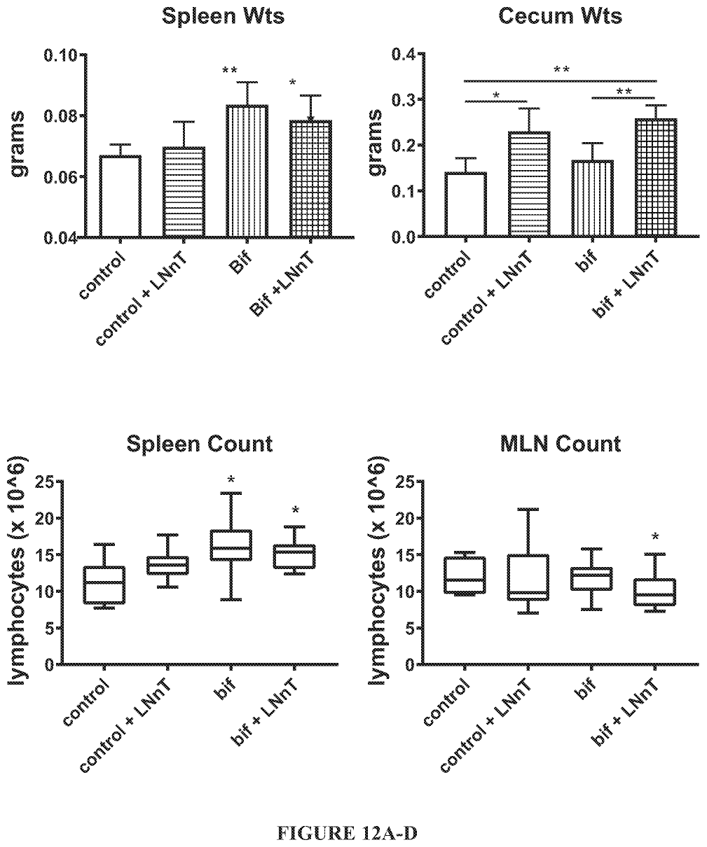

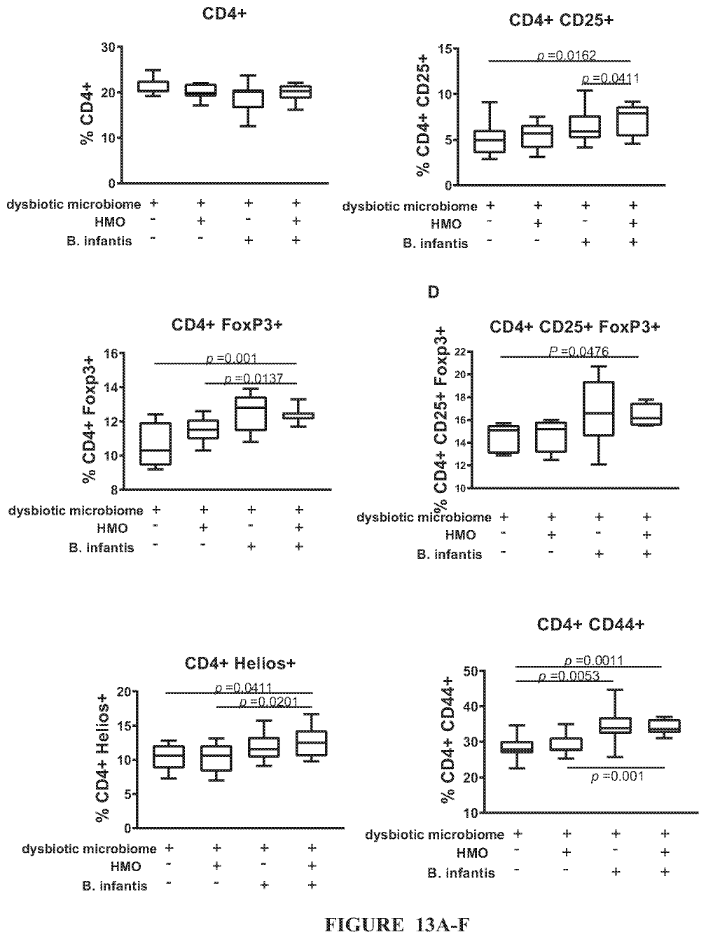

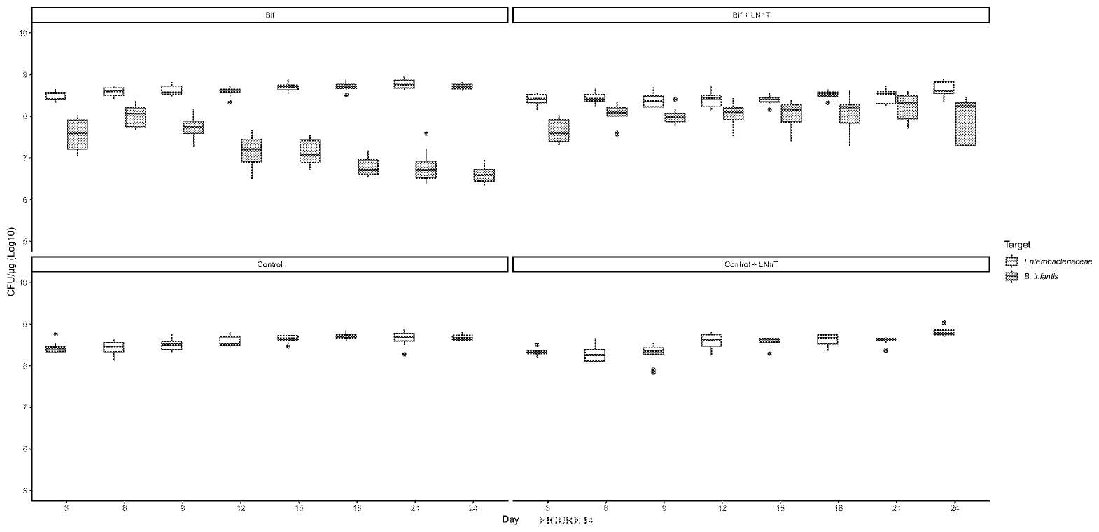

Germ-free mouse pups were weaned 3 or 4 weeks after birth and put on a polysaccharide-free mouse chow diet that contains Vitamin A. As soon as the mouse pups were weaned, they were gavaged with a dysbiotic (no Bifidobacterium sp. and high proteobacteria) human infant microbiome at Day 1 of the experiment. The mice were divided into 4 groups: control (placebo:placebo); control (B. infantis placebo) plus LNnT; B. infantis plus LNnT placebo; and B. infantis plus LNnT. All Groups received B. infantis or a placebo every 3 days by gavage. The LNnT or placebo was added to the drinking water for 21 days. At the end of the supplementation period, mice were necropsied. Mice that received B. infantis plus LNnT had greater spleen and cecum weight, consistent with immune cell expansion (FIGS. 12A and 12B). White blood cells were collected and were characterized and evaluated for T Regulatory cells, Th17 cells, CD4, CD8 cell populations of multiple origins using flow cytometry specific for CD4, CD25, Foxp3, Helios, Neuropilin, CD8, CD44, CD62L, Interferon gamma (IFNgamma), Interleukin 17 (IL-17), Transforming Growth Factor β (TGFβ), Interleukin 35 (IL-35), CD24, CD27, and/or CD38 (FIG. 13A-F depicts the analysis of lymphocyte populations in untreated and treated mice (A) total lymphocytes; (B) CD4+; (C) CD4+/CD25+Helios− FoxP3+; (D) CD4+/CD25+/Helios− FoxP3−; (E) CD4+/FoxP3+CD25−; (F) CD4+/Helios+CD25-Plasma is evaluated for cytokines and innate immune factors. Fecal samples were taken every day and evaluated using qPCR and/or 16s and/or shotgun sequencing for microbial colonization. FIG. 14 depicts the colony forming units of B. infantis and enterobacteria in mice after 21 days. Ileum and colon were collected for histopathology, qRTPCR and proteomics. Mice that received B. infantis plus LNnT were found to have a greater number of naïve B cells ready for antigen presentation. They also were found to have a thicker mucus layer on the surface of the epithelium and had more CD4+FoxP3+T regulatory cells compared to mice that remain dysbiotic.

### Example 3

### Prevention of Murine Peanut Allergy

Germ-free mouse pups are weaned 3-4 weeks after birth and put on a polysaccharide-free diet that is enriched for Vitamin A. As soon as the mouse pups are weaned, they are gavaged with a dysbiotic (no Bifidobacterium sp. and high proteobacteria) human infant microbiome at Day 1 of the experiment. The mice are divided into 4 groups: control, control plus LNnT, B. infantis, and B. infantis plus LNnT. Mice are given the composition for 21 days. All Groups receive B. infantis or placebo every 3 days by gavage. The LNnT or placebo is added to the drinking water for 21 days. Mice receive intragastric gavage of peanut extract on weekly at 5 mg/mouse for sensitization and then 25 mg/mouse at week 5 for challenge with 10 μg of Cholera toxin to elicit anaphylaxis. Control animals receive intragastric gavage with vehicle only. Anaphylaxis symptoms are evaluated, and then mice are necropsied. Plasma samples are collected for evaluation of histamine and IgE concentrations. White blood cells are isolated for PBMC characterization and evaluation of regulatory T cell population expansion using flow cytometry specific for CD4, CD25, Foxp3, Helios, Neuropilin, CD8, CD44, CD62L, IFNgamma, IL-17, and B cell populations, including Bregs and plasma cells using the following markers: IgM, CD5, CD24, CD19, CD19, CD20, CD34, CD38, CD45R, CD78, CD80, and CD138.

The administration and subsequent colonization of mice by B. infantis is found to reduce the inflammatory responses to the food antigen as compared to the control mice. B. infantis colonization will prevent anaphylaxis and/or reduction of allergy symptoms such as drop in core body temperature, increased allergen-specific IgE and IgG1, IL-4, and MCPT1 in the serum, expansion of mast cells in the jejunum, increased production of IL-13, edema and mast cell, eosinophils, and/or dendritic cell expansion, increase in IL-4 secreting CD4+ T cells in mesenteric lymph nodes (MLN) and spleen, decreased number of Foxp3+ Tregs in colon, spleen, and/or MLN, and allergic diarrhea.

### Example 4

### Reversal of Murine Peanut Allergy

Germ-free pups are weaned 3 weeks after birth and put on a polysaccharide-free diet. Mice are gavaged with a dysbiotic microbiome of Example 3 at Day 1. Mice receive intragastric gavage of peanut extract on day 1 and day 7 at 5 mg/mouse and then 25 mg/mouse at week 5 with 10 μg of Cholera toxin. Elicitation of reaction is performed at eight to ten weeks using intragastric gavage of 10 mg/mouse food antigen (e.g., peanut extract) with vehicle (PBS). At 10 weeks, mice of the test group are fed B. infantis, while control mice receive vehicle gavage only. All mice have access ad labium to LNnT-infused drinking water for a period of 21 days. The mice are re-challenged with the food antigen via intragastric gavage of 10 mg/mouse peanut extract with vehicle (PBS). Anaphylaxis symptoms are evaluated, and then mice are necropsied. Plasma samples are collected for evaluation of histamine and IgE concentrations. White blood cells are isolated for PBMC characterization and evaluation of regulatory T cell population expansion using flow cytometry specific for CD4, CD25, Foxp3, Helios, Neuropilin, CD8, CD44, CD62L, IFNgamma, IL-17. The administration and subsequent colonization of the mice by B. infantis reduces the inflammatory responses to the food antigen in animals that previously showed a sensitivity to the food antigen as compared to the control mice.

### Example 5

### Prevention of Human Peanut Allergy in Infants

Infants 2-4 months of age at risk of allergy are recruited and their stool is screened for their Bifidobacterium infantis status (Bifidobacterium infantis abundance). The infants are divided into 2 groups: high Bifidobacterium (desirable infant gut microbiome) and low Bifidobacterium (dysbiotic infant gut microbiome). Low Bifidobacterium stool samples are determined to be low Bifidobacterium or dysbiotic, if their levels of Bifidobacterium are less than a threshold of 10⁸ CFU/ug DNA and/or Bifidobacteriaceae family is less than 35% of the total infant gut microbiome as measured by a next generation sequencing, such as 16S RNA sequence. Conversely, a high Bifidobacterium sample or desirable infant gut microbiome has a threshold of 10⁸ CFU/ug DNA or greater and/or Bifidobacteriaceae family is greater than 35% of the total infant gut microbiome. Once the infants are screened, the dysbiotic group is randomized into a placebo arm or a supplemented arm receiving activated B. infantis, LNnT and retinol. Infants are fed the supplement or placebo for 16 weeks. At 12 weeks, peanut extract is introduced to the diet for 3 days to infants who are at least 4 months old. Blood samples are taken at 12 weeks, 16 weeks, 20 weeks, 24 weeks, 36 weeks, 40 weeks to analyze TReg cells and IgE. At 9 months, the infants are brought in for a peanut challenge. The incident of allergy between groups is analyzed. Improved tolerization of peanut extract identified in the B. infantis and LNnT group lead to decreased allergic reactions including significantly decreased concentration of peanut extract specific IgE compared to the dysbiotic group.

The administration and subsequent high-level of colonization of the infants by B. infantis reduces the inflammatory responses to the food antigen as compared to the control group with a low-level of B. infantis colonization.

### Example 6

### Reversal of Peanut Allergy in Children Under 3 Years of Age

Peanut allergic infants 9-18 months of age are recruited to participate in an immunotherapy protocol to reverse their known peanut allergy. Infants are put on a diet containing an oligosaccharide diet component consisting of 15 grams/day of formulation containing 50% LNnT and 25% LNT and 25% GOS, in addition to a supplement of B. infantis (4 billion CFU/serving) twice a day and a source of dietary ii-cryptoxanthin, such as oranges to consume 12 mg/day (expected yield 500 ug/day of retinol) for 12 weeks. At 8 weeks; a low dose of a peanut extract is added to the diet under medical supervision for one week. The infants are brought back at week 16 for a peanut challenge.

The administration and subsequent colonization of the infants by B. infantis may reduce the inflammatory responses to the food antigen.

### Example 7

### Prevention of Atopic March

Infants are enrolled at birth and randomized into 4 groups: 1) placebo; 2) B. infantis EVC001 with exclusive human milk diet; 3) B. infantis EVC001 and exclusive feeding with formula containing 8 g/L LNT; and 4) B. infantis EVC001 and exclusive formula feeding with 8 g/L released N-glycans from bovine whey proteins. Infants are fed B. infantis EVC001 until it has stably colonized to >10⁶ for 100 days of life. After 100 days infants continue with the same feeding strategy as they started until 6 months of age without supplementation of B. infantis EV001. Stool samples are collected weekly for the duration of the study period and 1 blood sample is collected per month for the duration of the study. After the 100 day high B. infantis period, infants are followed for the next year. Fecal samples will be analyzed for metagenomics, metabolomics, qPCR sIgA, and fecal cytokines. Blood sample analysis will include immune cell characterization, cell function analysis, metabolomics, epigenetics, metagenomics, innate immune and acute phase protein quantification, cytokine (notably IL-4, and IL-13), and IgG1 and IgE antibody quantification (including autoantibodies). Outcomes may include one or more of the following: improved Treg/Th17 ratio compared to placebo; Increase in T reg cells or decrease in Th17 cells; increased number of B regulatory cells, decreased cytokine production, decreased innate immune factor levels, decreased acute phase protein release; increased vaccine response or titer, decreased autoantibodies, and/or decreased inflammation.

Therapeutic outcomes include decreased atopic wheeze, asthma, eczema. Reduced incidence of atopic diseases including atopic wheeze, asthma. Other autoimmune and inflammatory conditions could be evaluated such as type I diabetes, Inflammatory bowel disease at latter follow-up period out to age 6 of life.

### Example 8

### Prevention of Type 1 Diabetes in Mice

Germ-free non-obese diabetic (NOD) mouse pups are weaned 3 weeks after birth and put on a polysaccharide-free diet that contains Vitamin A. As soon as the mouse pups are weaned, they are gavaged with a dysbiotic (no Bifidobacterium sp. and high proteobacteria) human infant microbiome at Day 1 of the experiment. The mice are divided into 4 groups: control, control plus LNnT, B. infantis, and B. infantis plus LNnT. Mice are given the composition for 21 days. All Groups receive B. infantis or a placebo every 3 days by gavage; the LNnT or placebo is added to the drinking water. Additional mice are divided into 2 groups These groups are gavaged with a healthy human infant microbiome (Bifidobacterium species) at day 21 of the experiment. The mice are then either fed placebo or LNnT in their drinking and receive vitamin A in their chow. Mice are monitored for diabetes with weekly tail vein blood glucose measurements and euthanized following two consecutive daily readings of >14 mmol⁻¹. At necropsy, lamina propria, spleen, mysenteric lymph nodes, and blood samples are collected for PBMC characterization and evaluation of expansion using flow cytometry specific for CD4, CD25, Foxp3, Helios, Neuropilin, CD8, CD44, CD62L, IFNgamma, IL-17, IgM, CD3, CD5, CD24, CD19, CD19, CD20, CD34, CD38, CD45R, CD78, CD80, and CD138. Plasma is evaluated for cytokines and innate immune factors. Fecal samples are taken every day and evaluated using qPCR and/or 16s and/or shotgun sequencing for microbial colonization. Ileum and colon are collected for histopathology, mucin content qPCR and proteomics.

The administration and subsequent colonization of the mice by B. infantis reduces the incidence of the development of diabetes mellitus as compared to the control mice.

### Example 9

### Nutritional Intervention Study to Evaluate the Effect of B. Infantis on the Development of Autoimmune and/or Allergic Disease (T1D) in Breastfed Infants

Emerging evidence suggests that B. infantis colonization in breastfed human infants has decreased over the last 50 years, due in part to the use of antibiotics and decreased rates of breast feeding. The lack of colonization with B. infantis in infants during this period has been associated with increased rates of childhood-onset autoimmune (e.g. T1D, celiac disease) and allergic (e.g. eczema, asthma) disease. It is hypothesized that restoring high rates of predominant intestinal colonization in infants with B. infantis may improve Treg cell level and/or activity and decrease the rates of childhood-onset autoimmune and allergic disease.

Administration of activated B. infantis to breastfed neonates and infants leads consistently to high levels of B. infantis in the stool. This study will investigate whether induction of B. infantis colonization of breastfed infants with B. infantis EVC001 decreases the risk of developing T1D, as measured by seroconversion to multiple pancreatic islet cell autoantibodies (e.g., stage 1 T1D) at 36 months of age.

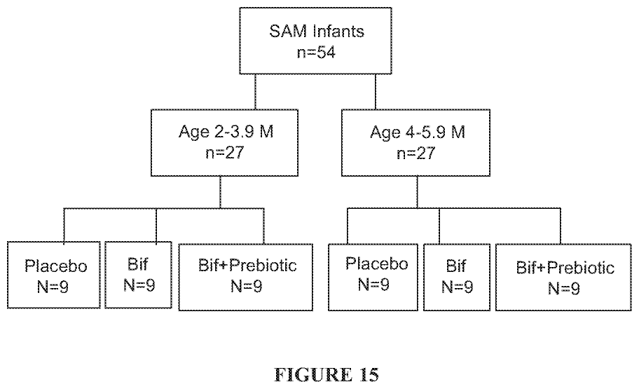

This is a randomized, double-blinded, placebo-controlled, parallel group study design (See FIG. 15). After the parent/guardian(s) sign the informed consent, infants will be screened for all the eligibility criteria, and if found eligible will be enrolled in the study. Eligible infants must first undergo an HLA genotyping from a cord blood sample collected at birth. Based on their HLA genotype they will be either placed in group 1 (low risk by HLA genotyping) and group 2 (high risk by HLA genotype). Each group will be randomly assigned to the placebo or treatment groups. After baseline assessments are collected, infants will be randomized to receive B. infantis EVC001 or placebo once daily for 12 months. Parent/guardian(s) of the infants will be instructed to maintain breastfeeding (at least 1 feed/day) for at least 6 months, and up to 12 months if possible during the treatment period. Following the 12-month treatment period, individuals will be followed at regular intervals with study visits and assessments. Individuals will be followed until a sufficient number of events (seroconversion to multiple (2) islet autoantibodies) are accumulated to achieve the planned power for the final analysis.

### Schematic Overview of the Study

Participants will receive B. infantis EVC001 or placebo once daily for a total of 12 months, which will be delivered at home by a parent/guardian or other caregiver. A single dose sachet (containing 8 billion CFU of activated B. infantis EVC001+lactose), or matching placebo sachet will be administered daily. At the time of dosing, a single sachet of B. infantis EVC001 or placebo will be mixed with a few tablespoons of expressed breast milk, which will then be delivered to the infant's mouth at the time of initiation of the feed.

Individuals who are found to seroconvert to multiple autoantibodies during the course of the study will be monitored regularly for evidence of asymptomatic dysglycemia (Stage 2 T1D) and symptomatic dysglycemia (Stage 3 T1D). Any participants who are found to have stage 2 or 3 T1D will be monitored and treated according to the standard of care through the course of the study.

The total duration of treatment will be 12 months for all groups. All mothers (regardless of group) will be encouraged and supported to continue breast feeding for at least 6 months, and if possible through the entire 12-month treatment period.

EFFICACY EVALUATIONS: The primary efficacy evaluation will be seroconversion of 2 out of 4 islet autoantibodies (IAA, GAD65, IA2 & ZnT8). Secondary efficacy evaluations will include rates of development of eczema and infantile colic. Exploratory efficacy evaluations include changes in body weight and seroconversion to positivity for tissue transglutaminase autoantibodies.

BIOMARKER EVALUATIONS will include Serum/plasma IgE—total and antigen specific (cat, dog, egg, cow's milk, house dust mite, timothy grass, birch and peanut), fecal microbiome analysis (shotgun metagenomics) and fecal metabolomics.

OTHER EVALUATIONS include DEXA scan for body composition, Health questionnaire(s) to track breast feeding, sleep patterns, and colic symptoms, Disease screening questionnaires to gather evidence of development of eczema, allergic rhinitis, asthma, or other allergic disease, and Skin prick testing of a panel of allergens.

Blood samples are collected at baseline, 3, 6, 9, and 12 months postpartum for evaluation of peripheral blood mononuclear cell characterization (including regulatory T cells), islet cell autoantibodies, vaccine response.

The colonization of infants with B. infantis increases TReg cell numbers and decreases the rates of childhood-onset autoimmune and allergic disease.

### Example 10

### Nutritional Intervention Study to Evaluate the Effect of B. Infantis on the Development of Autoimmune and/or Allergic Disease (T1D) in Formula Fed Infants

To determine the effectiveness of altering the development of T1D in infants, the study design of Example 8 was modified to recruit infants 0-1 month of age who are exclusively formula fed. These infants are fed a composition comprising LNT, activated B. infantis, Vitamin A and D. A single serving composition of MCT oil, with Vitamin A and D and B. infantis is given once daily for the first 6 months of life. The oil serving is added to a small volume of reconstituted infant formula and given in a single serving. The total intake of LNT is calculated based on a daily concentration of 12 g/L. The LNT supplement is packaged to provide the appropriate amount of LNT per 2 oz of formula and every bottle receives a dose of LNT depending on the volume required (i.e., for an 8 oz bottle 4 sachets of LNT would be used). The effect of this treatment is evaluated at 2 months, 6 months, 12 months, 18 months, 24 and 36 months.

One skilled in the art will recognize that examples 8 and 9 provide options for breast feeding and formula feeding, the exact timeline for supplementation and the composition may be modified to alternative embodiments described herein as part of the invention. These particular examples are for illustration purposes only. In other examples one could study LNT and vitamin A supplementation compared to placebo.

### Example 11

### Improved Vaccine Response in Immunosenescent Individuals

Older adults are screened for their antibody titers to a past pneumonia vaccine. The at risk population (low antibody titer/poor immune function) is divided into 3 groups, a placebo control group and a group that receives protein containing threonine, Vitamin A, a combination of LNT/GOS and B. infantis and a group receiving Vitamin A and LNT. Individuals take the supplement once daily for a total of 8 weeks (4 weeks before and 4 weeks after vaccine). At week 4, the individuals are given the DTap vaccine. Antibody titers are evaluated 4 weeks and 3 months post vaccination.

Adults receiving the composition including B. infantis, LNT and vitamin A are expected to show higher antibody titers than adults in the placebo control group and can also be compare to LNT and Vitamin A alone.

### Example 12

### Prevention and/or Treatment of SAM

Malnutrition is an ever-present issue worldwide. It is estimated that over 18 million children under the age of 5 are affected by the most extreme form of undernutrition, severe acute malnutrition (SAM). Children with SAM are twelve times more likely to die than well-nourished children. Infectious morbidity is common among survivors. Causes of malnutrition are typically understood to be related to chronic poverty, lack of access to nutritious foods, lack of appropriate breastfeeding, repeated infections and poor hygiene. Although improvements can be made by providing malnourished children with adequate nutrition, there is still a population refractory to current therapeutic interventions. Research indicates that gut microbes are related to undernutrition and that children with SAM have gut dysbiosis that mediates some of the pathology of their condition. The standard of care in these children should be reinforced by an intervention that corrects the gut dysbiosis, barrier function and B and T cell function, improves weight gain during nutritional rehabilitation, and reduces infectious morbidity.

In a Single-blind RCT, stratified randomization study, the T cell response following intervention to modulate the infant gut through administration of B. infantis, LNnT, and a nutrient supplement containing Vitamin A for 28 days will be measured (FIG. 15). The target population is first stratified into 2 groups as shown in FIG. 2. Group 1 consists of SAM infants between 2 and <6 months old with severe acute malnutrition randomized to three treatment arms upon completion of stabilization phase of treatment of SAM; SAM will be defined as weight-for-length <−3 Z. Group 2 consists of non-malnourished infants (WLZ≥−1) <6 months old who are hospitalized for treatment with antibiotics for infection. Infants in this group will be receiving at least 50% of their nutritional intake from breast milk in order to be eligible for enrollment. Exclusion criteria (for both groups): Septic shock or very severe pneumonia requiring assisted ventilation or acute kidney injury on admission, congenital defects interfering with feeding such as cleft palate, chromosomal anomalies, jaundice, tuberculosis, presence of bilateral pedal edema, maternal antibiotic usage for breastfeeding infants (current antibiotic use). Group 1 (SAM) additional exclusion criteria: Infants receiving ≥75% of nutrition from breast milk. Group 2 (non-malnourished) additional exclusion criteria: Infants receiving <50% of nutrition from breast milk.

The microbiome response to probiotic supplementation (with and without prebiotics) in patient population Group 1 will be monitored to justify a larger study of clinical outcomes. Additionally, non-malnourished infants who are hospitalized for infectious conditions face challenges related to dysbiosis caused by antibiotic usage. In Group 2, the ability of activated B. infantis EVC001 to rescue the microbiome of primarily breastfed non-malnourished infants will be evaluated.

Stool samples are collected for evaluation of B. infantis colonization and markers of mucosal epithelial monolayer integrity and inflammation. Blood samples are collected at baseline and at 28 days for evaluation of peripheral blood mononuclear cell characterization (including regulatory T cells, B cell and plasma cells) and vaccine response. Babies are evaluated for symptoms or severe acute malnutrition or enteric infections, including the development of sepsis.

Children receiving B. infantis show improved B cell and plasma cell profiles, vaccine responses, better weight gain, better ratio of lean to fat body mass and lower incidence of symptoms of acute malnutrition and expansion of TReg cells.

### Example 13

### In Vitro Identification of Antigen Recognition Sites on B. Infantis Important for Tolerance and Treg Cell Expansion

To determine the important cell surface components required for tolerance enhancement by B. infantis when interacting with host immune cells, we evaluate the exopolysaccharides, proteins, and/or genes involved in the interaction between B. infantis cell surface and the dendritic cells to initiate development/expansion of TReg cell populations.

Step 1. Identification of Exopolysaccharide (EPS) Secretion in Activated B. infantis.

Secretion of EPS by activated B. infantis will be determined by electron microscopy following the methods from Schiavi et al. (2016) AEM: 82:7185. B. infantis cells are grown on liquid media of yeast extract-free MRS containing lactose (unactivated cells) or human milk oligosaccharides (activated cells) as the sole carbon source for 48 hours. After culture in MRS medium, bacteria are gently rinsed in PIPES (piperazine-N,N-bis-2-ethane sulfonic acid) buffer (0.1 M, pH 7.4) and fixed in 2.5% glutaraldehyde resuspended in PIPES buffer for 5 min. Samples are rinsed twice (2 min each time) in PIPES buffer and postfixed with 1% osmium tetroxide in 0.1 M PIPES buffer (pH 6.8) for 60 min in the dark. Samples are then washed three times in miliQ water (2 min each wash) before being dehydrated through an ethanol series (50, 70, 96, and 100%) for 5 min each step. All fixation and washing steps are carried out at room temperature. Following dehydration, samples are then critically point dried in and coated with 10 nm of gold/palladium (80/20). Bacterial preparations are examined using a scanning electron microscope (SEM).

Step 2. Extraction and Quantification of Exopolysaccharide (EPS) Secretion in Activated B. infantis.

For EPS secretion to be determined in B. infantis, cells will be grown on agar plates of yeast extract-free MRS containing lactose (unactivated cells) or human milk oligosaccharides (activated) as the sole carbon source for 48 hours. After 48 hours, EPS is extracted according to Altmann et al. 2016 with a few modifications. Briefly, cells are resuspended in phosphate-buffered saline solution and mixed with three volumes of cold absolute ethanol to a 80% (v/v) final concentration, followed by precipitation in ethanol solution overnight at 4° C. The precipitate is removed with a spatula and resuspended in miliQ water. Purification from contaminants and residual ethanol can be performed on C18 cartridges connected to a vacuum manifold. The eluted EPS is filtered through a 0.45 μm syringe filter and quantified at 490 nanometers according to Matsuko et al. 2005 with a phenol-sulfuric acid colorimetric method. Levels of EPS secreted in activated versus inactivated B. infantis are quantified in nmols/well.

### Step 3. Characterization of the Composition of the Exopolysaccharide (EPS).

EPS composition will be elucidated with Matrix-Assisted Laser Desorption/Ionization Time-Of-Flight Mass Spectrometry (MALDI-TOF MS) following the methods from Gonzalez-Gil et al. 2015 with modifications. EPS containing samples will be mixed at a 1:1 ratio with 20-40 mg/ml of 2,5-dihydroxybenzoic acid (DHB) matrix dissolved in 30% (v/v) acetonitrile, 0.1% (v/v) trifluoroacetic acid in miliQ water. A 2 μl volume of sample/matrix mixture is then spotted on a MALDI target plate for analysis. Spectra will be compared to reference polysaccharides (dextran, gellan, xanthan and alginate) prepared in the same manner as experimental samples. MALDI-TOF MS will be run on positive mode following the manufacturer's recommendations for polysaccharide characterization.

### Step 4. Characterization of Solute Binding Proteins and Other Signal Molecules in Activated and Unactivated Cells.

In addition to EPS, secretion of proteins and signal molecules (e.g., glycolipids) to the extracellular space will be analyzed. B. infantis cells are grown in 500 ml of MRS containing lactose (unactivated cells) or human milk oligosaccharides (activated) as the sole carbon source for 48 hours. The cell yields are normalized by optical density and centrifuged out at 6012×g. The supernatant is then filtered through a 0.2 μm syringe filter and divided into 2 fractions. One fraction will be used for protein collection, and the other fraction will be used for lipid and glycolipid identification. In the first fraction, proteins will be concentrated with an Amicon Ultra-0.5 ml centrifugal filters, 3K cut-off, and separated on a 12% SDS-PAGE according to Ortega Ramirez et al 2018 and analyzed on a nano Liquid Chromatograph-iontrap Mass Spectrometer connected to a C18 column. Spectral counts will then be obtained with IdPicker (Ma et al. 2009) using the Myrimatch search engine algorithm (Tabb et al. 2007). To determine the relative protein abundancy, the log 2(spectral counts) will be normalized using the central tendency of the mean. The protein relative abundances will be compared between activated and inactivated B. infantis to seek for statistical significance. The second fraction will be extracted with ethyl acetate and dried under a nitrogen gas stream, according to Orteag Ramirez et al. International Biodeterioration & Biodegradation (2018) 130: 40-47.

To seek for additional signal molecules (e.g. glycolipids or lipids), experimental samples (activated versus inactivated) will be analyzed on a MALDI-TOF MS calibrated with maltooligosaccharides. Calibrators and samples will be mix on a 2:1, DHB matrix/sample ratio using the sandwich technique. Mixture will then be spotted on a MALDI target plate. Signal molecules will be analyzed on a positive mode following the instrument manufacturer's recommendations.

### Step 5. Measure the Activation of Dendritic Cells in Culture.

An equal number of cells from each group (activated, unactivated) are used. The cells are incubated with a cell culture of dendritic cells. After overnight incubation at 37C, the supernatant is gently removed from the culture dish. The dendritic cells are washed to remove any remaining culture broth and any B. infantis not bound to the dendritic cell. MRS is added to the culture dish, and the plate vigorously shaken to displace any bound B. infantis. The CFU/ml of B. infantis in the activated group is compared to the un-activated group. This provides an initial screening step to look at key structures that are induced during activation, such as the solute binding proteins. Dendritic cells are examined for activation of pathways known to be important in antigen recognition including pattern recognition, receptor expression, cytokine production, MHC class II, and DECTIN-1. In other experiments, dendritic cells are co-cultured with activated B. infantis overnight in the presence of anti-inflammatory cytokines, including IL-10 before they are separated. Treated dendritic cells are then co-cultured with naïve T cells in the presence of retinoic acid overnight at 37° C. T cells are characterized for specific markers of differentiation, including T reg markers included elsewhere.

In another set of experiments B. infantis, cells will be grown on agar plates of yeast extract-free MRS containing lactose (unactivated cells) or human milk oligosaccharides (activated) as the sole carbon source for 48 hours. The optical density is measured to ensure equal number of cells in the mixtures. The exopolysaccharide is gently removed from one culture tube by resuspending the cells for 1 hour in phosphate saline buffer containing ribonuclease and deoxyribonuclease (1 μg/mL and 5 μg/mL, respectively) Whole cells are harvested by centrifugation (20,000×g at 4° C. for 10 min) and resuspended in a chemically defined fresh media (e.g. RPMI media). The exopolysaccharide-free cells are added to a culture of dendritic cells to determine the level of antigen recognition compared to cells with exopolysaccharide.

### Step 6. Genomic Analysis

The transcriptome, that is the full range of messenger RNA(mRNA) molecules expressed by activated-EPS producing B. infantis will be compared to that of unactivated non-EPS producing B. infantis using standardized nucleic acid extraction and sequencing methods, e.g., Garber et al. 2001, Nature Methods, 8:469. The biochemical pathways for exopolysaccharide production in activated cells are then elucidated by interpreting the transcriptome data using metabolic pathway prediction tools, e.g., Kamburov et al. 2011 Bioinformatics, 27:2917. Data supplied to the prediction algorithm can be further enhanced by generating and integrating metabolomics and/or proteomics data.

### Example 14

### Isolation of B. Infantis Cell Wall as an Ingredient to Augment Treg Cell Production

A food grade process is used to produce stable cell wall fragments from B. infantis that include SBP and/or exopolysaccharides. The B. infantis is grown on an activating agent (immunel, see International Patent Publication No. WO 2016/065324) to a yield of at least 100 billion CFU/ml. The cells are harvested and the supernatant removed. A solution containing the cells is lyzed using an ultrasonic technique to disrupt the cells. The mixture is acidified and heated to 35° C. to precipitated the membranes and separate them from the rest of the lysed cell debris

The precipitated membrane fractions are added to an oil and fed to naïve mice for 7 days with vitamin A, and the induction of TReg cells are analyzed by isolation of White blood cells for PBMC characterization and evaluation of regulatory T cell population expansion using flow cytometry specific for CD4, CD25, Foxp3, Helios, Neuropilin, CD8, CD44, CD62L, IFNgamma, IL-17 at day 28.

Mice fed the B. infantis composition show an increased white blood cell and T cell population levels.

### Example 15

### B. Infantis Metabolites Protect Human Intestinal Epithelial Cells from Pathogen Induced Inflammation

Little is known about the anti-inflammatory effects B. infantis-derived metabolites have on intestinal epithelial cells and how they might protect and maintain mucosal integrity in the infant gut. To investigate whether metabolites from B. infantis grown on HMO and synthetic HMOs can provide protective effects against pathogen-induced inflammation and maintain mucosal integrity compared to other commensal strains that have been previously characterized. B. infantis, B. breve, B. bifidum, and B. longum were grown in media containing pooled HMOs, synthetic Lacto-N-neotetraose (LNnT), or fructo-oligosaccharide (FOS, a readily available oligosaccharide commonly used in baby formulas). Four Lactobacillus plantarum strains were also grown in HMO, LNnT, and FOS media. Spent supernatant was collected and filtered after 48 hours of growth (when the bacteria reached the stationary phase) and evaluated for the remaining oligosaccharide concentration. Human intestinal epithelial cells (IECs; HT-29) were grown to confluency in 96-well plates and exposed to cell medium containing 15% of spent bacterial supernatant for 1 hour at 37° C. before media was removed and IECs monolayers were challenged with media containing lipopolysaccharide (LPS) from E. coli 0111:B4. After overnight incubation, cell supernatant was analyzed by ELISA for reductions in pro-inflammatory cytokines, including IL-8 and TNF-alpha. The amount of mucus produced is compared between the groups.

First, growth curves indicated that B. infantis had a selective growth advantage when grown in HMOs compared to other Bifidobacterium and Lactobacillus strains. These data further showed that multiple strains of Bifidobacterium and Lactobacillus grew very well using FOS alone as a carbon source. Furthermore, HPLC data confirmed that B. infantis growth advantage was due to its ability to utilize HMOs as a carbon source since very low concentrations of pooled or synthetic HMOs could be measured in the spent supernatant. Conversely, high concentrations of pooled and synthetic HMOs remained in the other strains of Bifidobacterium and Lactobacillus strains, which confirms HMOs provide selective growth for B. infantis. All strains tested were able to readily use FOS as a carbon source. Furthermore, IECs exposed to spent supernatant from B. infantis grown on pooled or synthetic HMOs or FOS for 1 hour prior to pathogenic bacterial challenge significantly reduced proinflammatory response compared to medium alone (P=0.015, 0.01, and 0.0005, respectively). Moreover, this protective effect was unique to B. infantis compared to other Bifidobacterium and specific Lactobacillus strains used in the study.

These data demonstrate that metabolites produced by B. infantis deliver direct protective effects against pathogen-induced inflammation in the intestinal mucosa that is unique to this strain of bacteria.

### Example 16

### Immune Development in C-Section Infants

Approximately 25 mother-infant dyads are recruited to provide a cord blood sample and two infant blood samples, one on day 0-4 and another at 3 months old (between 84 and 104). Markers of immune function will be analysed and compared to the development of the gut microbiome.

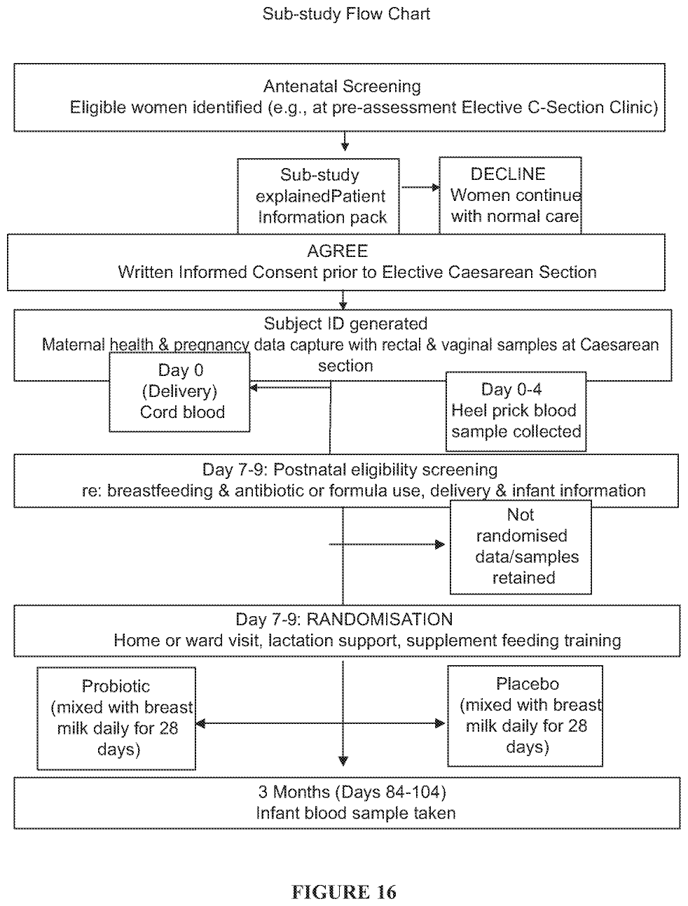

The study design is described in FIG. 16. The change in levels of immune cells and markers from baseline (Day 0-Day 4) to three months (Day 84-Day 104) will be measured. The correlation between gut microbiota composition and abundance and levels of immune cells and markers may also be measured. The differences between B. infantis and placebo supplementation on levels of immune cells and markers in breast-fed infants.

The differences between B. infantis and placebo supplementation on vaccine response (antibody titers). Blood samples are collected at baseline and month-three postpartum for evaluation of peripheral blood mononuclear cell characterization (including regulatory T cells) and vaccine response.

The effect of supplementation on colic, diaper rash and sleep will be evaluated. It is expected that there will be reduced inflammation, increased Tregs, decreased Th17 and IL-17 level, an improvement in diaper rash and/or colic.

This example provides an illustration of a study design that can evaluate the effects of any composition on the immune system of C-section infants or other groups in need of correcting dysbiosis. One skilled in the art will recognize that compositions with and without Vitamin A or D, or compositions with different types of OS can be delivered and evaluated using this or similar study design.

All publications, patents, and published patent applications mentioned in this specification are herein incorporated by reference, in their entirety, to the same extent as if each individual publication, patent, or published patent application was specifically and individually indicated to be incorporated by reference.

1. A composition comprising Vitamin A, or a Vitamin A derivative or metabolite thereof, an oligosaccharide (OS), and optionally Bifidobacterium or activated Bifidobacterium.

2. The composition of claim 1, wherein the vitamin A is retinol, retinal, retinoic acid, a provitamin A carotenoid, or a combination thereof, and/or
wherein the provitamin A carotenoid is alpha-carotene, beta-carotene, gamma-carotene, xanthophyll beta-cryptoxanthin, or a combination thereof.

3-4. (canceled)

5. The method of claim 55, wherein the method comprises delivering 1-10,000 International Units vitamin A per day; or
wherein the method comprises delivering 1-2,000 International Unites vitamin A per day.

6. (canceled)

7. The composition of claim 1, wherein the composition comprises 1-100 pmol/l vitamin A; or
wherein the composition comprises about 5, 10, 15, 20, 25, 30, 35, 40, 45, 50, 55, 60, 65, 70, 75, 80, 85, 90, 95, or 100 umol/l vitamin A; or
wherein the composition comprises about 1-100, 5-50, 25-75, 10-100, 30-60, or 75-100 pmol/l vitamin A.

8. (canceled)

9. The composition of claim 1, wherein the oligosaccharide (OS) comprises a one or more oligosaccharides with 2 to 10 residues (DP2-10 oligosaccharides) and/or
wherein at least one of the oligosaccharides has a Type I or II core, or at least one of each; or
wherein the oligosaccharide (OS) comprises a one or more of DP3-10 oligosaccharides having 3-10 sugar residues structures found in mammalian milk; or
wherein the oligosaccharide is derived from a plant, fungus, animal, insect, or crustacean; or
wherein the oligosaccharide is sourced from a fungi, insect, crustacean or plant-derived oligosaccharide, optionally pre-digested from the source polysaccharide, and/or
wherein the plant-derived oligosaccharides are between 2 and 10 sugar residues (DP2-DP10), between 3 and 10 sugar residues (DP3-DP10), between 5 and 10 sugar residues (DP5-DP10), or up to DP20, or greater than DP30; or
wherein the plant-derived oligosaccharide is from orange peels, cocoa hulls, olive pomace, tomato skins, grape pomace, corn silage, or a mixture thereof; or
wherein the oligosaccharide is from carrots, peas, broccoli, onions, tomatoes, peppers, rice, wheat, oats, bran, oranges, coca, olives, apples, grapes, sugar beets, cabbage, corn, soy, shrimp, mushroom, or a mixture thereof; or
wherein the oligosaccharide is pre-digested polysaccharides from orange peels, shrimp, mushroom, cocoa hulls, olive pomace, tomato skins, grape pomace, corn silage or a mixture thereof; or
wherein the oligosaccharide comprises galactooligosaccharide (GOS), fructooligosaccharide (FOS), or xylooligosaccharide (XOS); or
wherein the oligosaccharide is a mammalian milk oligosaccharide (MMO), and/or
wherein the mammalian milk oligosaccharide (MMO) comprises oligosaccharide molecules found in human milk oligosaccharides (HMO), bovine milk oligosaccharides (BMO), bovine colostrum oligosaccharides (BCO), goat milk oligosaccharides (GMO), or a combination thereof, or
wherein the mammalian milk oligosaccharide (MMO) comprises one or more selected from lacto-N-biose, N-acetlylactosamine, lacto-N-triose, lacto-N-neotetrose, lacto-N-tetrose, N-acetyllactosamime, lacto-N-neotetraose, lacto-N-tetraose, fucosyllactose, lacto-N-fucopentose, lactodifucotetrose, sialyllactose, di sialyllactone-N-tetrose, 2′-fucosyllactose, 3′-sialyllactoseamine, 3′-fucosyllactose, 3′-sialyl-3-fucosyllactose, 3′-sialyllactose, 6′-sialyllactosamine, 6′-sialyllactose, difucosyllactose, lacto-N-fucosylpentose I, lacto-N-fucosylpentose II, lacto-N-fucosylpentose III, lacto-N-fucosylpentose V, sialyllacto-N-tetraose, their derivatives, or combinations thereof; or
wherein the oligosaccharide (OS) comprises a human milk oligosaccharide (HMO); or
wherein the oligosaccharide contains at least one Type II core; or
wherein the oligosaccharide contains at least one Type I core.

10-21. (canceled)

22. The composition of claim 1, wherein the composition provides a total dietary intake of oligosaccharide in an amount of 0.001-100 grams per day; or
wherein the composition provides a total oligosaccharide daily intake regardless of delivery form or dosing regime, in an amount of 0.1-50 grams per day; or
wherein the oligosaccharide is in an amount of 1-20 grams, 3-20 grams, or 5-10 grams per unit dose; or
wherein the oligosaccharide is in an among of 10, 15, 20, 25, 30, 35, 40, 45, or 50 grams; or
wherein the composition further comprises galactooligosaccharide (GOS), fructooligosaccharide (FOS), or xylooligosaccharide (XOS).

23-25. (canceled)

26. The composition of claim 1, wherein the Bifidobacterium is Bifidobacterium adolescentis, Bifidobacterium animalis, Bifidobacterium animalis subsp. animalis, Bifidobacterium animalis subsp. lactis, B. bifidum, Bifidobacterium breve, Bifidobacterium catenulatum, Bifidobacterium longum subsp. infantis, B. pseudocatanulatum, Bifidobacterium pseudolongum, activated B. longum subsp. infantis, or activated B. breve or a combination thereof, and/or
wherein the B. infantis has a functional H5 cluster, or
wherein B. infantis cell surface has increased exopolysaccharide and solute binding proteins; or
wherein the composition further comprises isolated B. infantis activated cell membranes comprising exopolysaccharides and/or solute binding proteins.

27-33. (canceled)

34. The composition of claim 1, wherein the composition comprises Bifidobacterium in an amount of 0.1-500 billion Colony Forming Units (CFU) per gram of composition; or
wherein the composition comprises Bifidobacterium in an amount of 1-100 billion Colony Forming Units (CFU) or 5-20 billion Colony Forming Units (CFU) per gram of composition or Colony Forming Units per μg DNA; or
wherein the Bifidobacterium is in an amount of 1, 5, 15, 20, 25, 30, 35, 40, 45, or 50 billion Colony Forming Units (CFU) per gram of composition.

35-46. (canceled)

47. The composition of claim 1, wherein the composition is a pharmaceutical composition, dietary supplement, nutritional product, food product, probiotic, and/or prebiotic, or
wherein the composition is formulated as a unit dose medicament; or
wherein the composition is formulated as a capsule, packet, sachet, foodstuff, lozenge, tablet, optionally an effervescent tablet, enema, suppository, dry powder, dry powder suspended in an oil, chewable composition, syrup, or gel; or
wherein the composition is a nutritional product, and/or
wherein the product is a food product, dietary supplement, infant formula, or pharmaceutical product or
wherein the composition is in the form of a dry powder or a dry powder suspending in an oil; or
wherein the composition is spray dried or freeze-dried, and/or
wherein the composition is freeze-dried in presence of a cryoprotectant, or
wherein the composition further comprises a stabilizer, and/or
wherein the stabilizer is a flow agent, or
wherein the stabilizer is a cryoprotectant, or
wherein the cryoprotectant is glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, taurine, mammalian milk oligosaccharides, polysaccharides or a combination thereof.

48-49. (canceled)

50. The composition of claim 1, wherein the composition further comprises an intact protein source or breakdown products rich in threonine, N-acetyl-thronine, gamma-glutamylthreonine, or a combination thereof.

51-54. (canceled)

55. A method for preventing and/or treating an autoimmune disease, preventing and/or treating an allergy, elevating and/or stimulating regulatory T-cell (Tregs) and/or B cells, increasing efficiency of antigen recognition in a subject, increasing efficiency of gene therapy and/or a vaccine, maintaining integrity of alimentary canal mucosal membrane during chemotherapy, protecting intestinal barrier integrity during chemotherapy or radiation treatment, stimulating mucin production, preventing and/or treating cancer comprising:
administering Vitamin A, or a Vitamin A derivative or metabolite thereof, or a source thereof, an oligosaccharide (OS), and optionally Bifidobacterium, and optionally a protein enriched for threonine and/or threonine, N-acetyl threonine and/or gammaglutamylthreonine, to a subject.

56-65. (canceled)

66. The method of claim 55, wherein the autoimmune disease is asthma, atopy, Type I diabetes, inflammatory bowel disease or celiac disease, and/or
wherein the inflammatory bowel disease (IBD) is ulcerative colitis (UC) or Crohn's Disease; or
wherein the subject is suffering from a hyperinflammatory gut or
wherein the allergy is a food allergy or atopy.

67-70. (canceled)

71. The method of claim 55, wherein the subject is a mammal, and/or
wherein the mammal is a human, cow, pig, rabbit, goat, sheep, cat, dog, horse, llama, or camel, or
wherein the mammal is an infant, or
wherein the mammal is a nursing infant mammal; or
wherein the subject is a human.

72-74. (canceled)

75. The method of claim 55, wherein the vitamin A is retinol, retinal, retinoic acid, a provitamin A carotenoid, or a combination thereof, and/or
wherein the provitamin A carotenoid is alpha-carotene, beta-carotene, gamma-carotene, xanthophyll beta-cryptoxanthin, or a combination thereof.

76-80. (canceled)

81. The method of claim 55, wherein the oligosaccharide (OS) comprises a one or more oligosaccharides with 2 to 10 residues (DP2-10 oligosaccharides), and/or
wherein at least one of the oligosaccharides has a Type I or II core, or at least one of each; or
wherein the oligosaccharide comprises a one or more of DP3-10 oligosaccharides having 3-10 sugar residues structures found in mammalian milk; or
wherein the oligosaccharide is derived from a plant, fungus, animal, insect, or crustacean; or
wherein the oligosaccharide is sourced from a fungi, insect, crustacean or plant-derived oligosaccharide, optionally pre-digested from the source polysaccharide, and/or
wherein the plant-derived oligosaccharides are between 2 and 10 sugar residues (DP2-DP10), between 3 and 10 sugar residues (DP3-DP10), between 5 and 10 sugar residues (DP5-DP10), or up to DP20, or greater than DP30; or
wherein the plant-derived oligosaccharide is from orange peels, cocoa hulls, olive pomace, tomato skins, grape pomace, corn silage, or a mixture thereof; or
wherein the oligosaccharide is from carrots, peas, broccoli, onions, tomatoes, peppers, rice, wheat, oats, bran, oranges, coca, olives, apples, grapes, sugar beets, cabbage, corn, soy, shrimp, mushroom, or a mixture thereof; or
wherein the oligosaccharide is pre-digested polysaccharides from orange peels, shrimp, mushroom, cocoa hulls, olive pomace, tomato skins, grape pomace, corn silage or a mixture thereof; or
wherein the oligosaccharide comprises galactooligosaccharide (GOS), fructooligosaccharide (FOS), or xylooligosaccharide (XOS); or
wherein the oligosaccharide is a mammalian milk oligosaccharide (MMO), and/or
wherein the mammalian milk oligosaccharide (MMO) comprises oligosaccharide molecules found in human milk oligosaccharides (HMO), bovine milk oligosaccharides (BMO), bovine colostrum oligosaccharides (BCO), goat milk oligosaccharides (GMO), or a combination thereof, or
wherein the mammalian milk oligosaccharide (MMO) comprises one or more selected from lacto-N-biose, N-acetlylactosamine, lacto-N-triose, lacto-N-neotetrose, lacto-N-tetrose, N-acetyllactosamime, lacto-N-neotetraose, lacto-N-tetraose, fucosyllactose, lacto-N-fucopentose, lactodifucotetrose, sialyllactose, disialyllactone-N-tetrose, 2′-fucosyllactose, 3′-sialyllactoseamine, 3′-fucosyllactose, 3′-sialyl-3-fucosyllactose, 3′-sialyllactose, 6′-sialyllactosamine, 6′-sialyllactose, difucosyllactose, lacto-N-fucosylpentose I, lacto-N-fucosylpentose II, lacto-N-fucosylpentose III, lacto-N-fucosylpentose V, sialyllacto-N-tetraose, their derivatives, or combinations thereof; or
wherein the oligosaccharide (OS) comprises a human milk oligosaccharide (HMO); or
wherein the oligosaccharide contains at least one Type II core; or
wherein the oligosaccharide contains at least one Type I core.

82-96. (canceled)

97. The method of claim 55, wherein the Bifidobacterium is Bifidobacterium adolescentis, Bifidobacterium animalis, Bifidobacterium animalis subsp. animalis, Bifidobacterium animalis subsp. lactis, B. bifidum, Bifidobacterium breve, Bifidobacterium catenulatum, Bifidobacterium longum subsp. infantis, B. pseudocatanulatum, Bifidobacterium pseudolongum, activated B. longum subsp. infantis, or activated B. breve or a combination thereof and/or
wherein the B. infantis has a functional H5 cluster, or
wherein B. infantis cell surface has increased exopolysaccharide and solute binding proteins; or
wherein the method further comprises administering isolated B. infantis activated cell membranes comprising exopolysaccharides and/or solute binding proteins.

98-113. (canceled)

114. The method of claim 55, wherein the method comprises administration of a composition,
wherein the composition is a pharmaceutical composition, dietary supplement, nutritional product, food product, probiotic, and/or prebiotic; or
wherein the composition is formulated as a unit dose medicament; or
wherein the composition is formulated as a capsule, packet, sachet, foodstuff, lozenge, tablet, optionally an effervescent tablet, enema, suppository, dry powder, dry powder suspended in an oil, chewable composition, syrup, or gel; or
wherein the composition is a nutritional product, and/or
wherein the product is a food product, dietary supplement, infant formula, or pharmaceutical product, or
wherein the composition is in the form of a dry powder or a dry powder suspending in an oil; or
wherein the composition is spray dried or freeze-dried, and/or
wherein the composition is freeze-dried in presence of a cryoprotectant; or
wherein the composition further comprises a stabilizer, and/or
wherein the stabilizer is a flow agent, or
wherein the stabilizer is a cryoprotectant; or
wherein the cryprotectant is glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, taurine, mammalian milk oligosaccharides, polysaccharides or a combination thereof.

115-116. (canceled)

117. The method of claim 55, wherein the elevation of the regulatory T-Cells (T_{regs}) results in the suppression of deleterious T-helper (Tₕ) cells; or
wherein the elevation of the regulatory T-Cells (T_{regs}) results in a decrease in inflammatory markers, and/or
wherein the inflammatory markers are IL-8, IL-6, TNF-a, IL-10 INF gamma, INF alpha, or a combination thereof, or
wherein the inflammatory markers are decreased by at least 5%, 10%, 15%, 20%, 25%, 30%, 35%, 40%, 45%, 50%, 55%, 60%, 65%, 70%, 75%, 80%, 85% or 90%; or
wherein function of immune system in the subject is enhanced subsequent to administration of said bacteria, said MMO, or both, and/or
wherein enhancement in the function of the immune system improves: vaccine response, mucosal innate or adaptive immunity, and/or improving homeostasis of innate and adaptive immunity systemically, or
wherein the function of the immune system is demonstrated by enhanced antibody titers in response to a vaccine, improved mucus production, increased T regulatory and B regulatory cell populations or increased sIgA production in gut leading to protection against pathogenic bacteria, and/or
optionally wherein the improvement or increase is statistically significant, or
wherein the increase is about 20, 30, 40, 50, 60, 70, 80, or 90%.

118-120. (canceled)

121. The method of claim 55, wherein the subject is already colonized by a Bifidobacterium species as measured by Bifidobacterium species CFU/gram of feces or CFU/μg DNA; or
wherein the subject is not colonized by a Bifidobacterium species as measured by Bifidobacterium species CFU/gram of feces.

122-123. (canceled)

124. The method of claim 55, wherein the dosage of retinoic acid, or a source thereof, oligosaccharide (OS), Bifidobacterium, or combinations thereof, is in an amount effective to maintain the Bifidobacterium level at least 10⁶, at least 10⁸ CFU/gram of feces or 10⁸ CFU/μg DNA or alternatively the relative abundance of Bifidoabcteriaceae in the microbiome of at least 10%, at least 20%, at least 30%, at least 50%, at least 60%, at least 70%, at least 80%, at least 90%, or alternatively, monitor by gene abundance of BLON 2175, BLON 2175 and/or Blon 2177; or
wherein upon administration of Bifidobacterium, colonization by Bifidobacterium species in the subject is increased by at least 1-10 CFU/gram of feces.

125. The method of claim 55, wherein the Bifidobacterium is administered to the subject on a daily basis comprising from 0.1 billion to 500 billion CFU of bacteria/day, or
wherein the Bifidobacterium administered on a daily basis can include from 1 billion to 100 billion CFU/day or from 5 billion to 20 billion CFU/day; or
wherein the Bifidobacterium is administered on a daily basis for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days out to 365 days; or
wherein the Bifidobacterium is administered on a daily basis for about 1-5 days, 6-10 days, 11-15 days, 16-20 days, 21-25 days, or 26-30 days.

126-128. (canceled)

129. The method of claim 55, wherein the oligosaccharide is administered in a solid or liquid form; or
wherein the oligosaccharide is administered in an amount of from about 0.1-50 g/day; or
wherein the oligosaccharide is administered in an amount of from about 2-30 g/day or 3-10 g/day; or
wherein the oligosaccharide (OS) comprises at least about 15%, at least 25%, at least 50%, at least 75% at least 95% of the subject's total dietary fiber.

130-131. (canceled)

132. The method of claim 55, wherein a first composition comprising retinoic acid and an oligosaccharide is administered to the subject and/or
wherein the first composition for use is administered several times a day, optionally 1-6 times a day, or
wherein the first composition for use is administered for at least 1-365 days; or
wherein a second composition for use comprising Bifidobacterium is administered to the subject, and/or
wherein the second composition for use is administered daily, or
wherein the second composition for use is administered for at least 1-365 days; or
wherein the first composition for use comprising retinoic acid, or a source thereof and an oligosaccharide is administered to a subject followed by the second composition for use comprising Bifidobacterium; or
wherein a third composition for use comprising retinoic acid, oligosaccharide, and Bifidobacterium is administered to a subject.

133-139. (canceled)

140. The method of claim 55, wherein the Vitamin A, or a Vitamin A derivative or metabolite thereof, or a source thereof, is administered several times a day for at least 1-30 days; or
wherein the oligosaccharide is administered several times a day for at least 1-30 days; or
wherein the Bifidobacterium is administered on a daily basis for at least 1-30 days; or
wherein the Vitamin A, or a Vitamin A derivative or metabolite thereof, or a source thereof, oligosaccharide, and Bifidobacterium are administered to the subject in a composition for use on a daily basis for at least 1-30 days; or
wherein the Vitamin A, or a Vitamin A derivative or metabolite thereof, or a source thereof and oligosaccharide are administered to the subject several times a day for at least 1-30 days followed by Bifidobacterium on a daily basis for at least 1-30 days.

141-158. (canceled)
